SEQUENCE LISTING (161 sequences)

SEQ ID NO: 1 - protein, 109 aa, Artificial Sequence
  Synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVL

SEQ ID NO: 2 - protein, 125 aa, Artificial Sequence
  Synthetic construct
EVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 3 - protein, 125 aa, Artificial Sequence
  Synthetic construct
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 4 - protein, 109 aa, Artificial Sequence
  Synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVL

SEQ ID NO: 5 - protein, 7 aa, Artificial Sequence
  Synthetic construct
GTNKRAP

SEQ ID NO: 6 - protein, 9 aa, Artificial Sequence
  Synthetic construct
ALWYSNLWV

SEQ ID NO: 7 - protein, 5 aa, Artificial Sequence
  Synthetic construct
TYAMN

SEQ ID NO: 8 - protein, 19 aa, Artificial Sequence
  Synthetic construct
RIRSKYNNYATYYADSVKD

SEQ ID NO: 9 - protein, 14 aa, Artificial Sequence
  Synthetic construct
HGNFGNSYVSWFAY

SEQ ID NO: 10 - protein, 15 aa, Artificial Sequence
  Synthetic construct
MMYCGGNEVLCGPRV

SEQ ID NO: 11 - protein, 15 aa, Artificial Sequence
  Synthetic construct
GYRWGCEWNCGGITT

SEQ ID NO: 12 - protein, 15 aa, Artificial Sequence
  Synthetic construct
GYLWGCEWNCGGITT

SEQ ID NO: 13 - protein, 8 aa, Artificial Sequence
  Synthetic construct
LSGRSDNH

SEQ ID NO: 14 - protein, 8 aa, Artificial Sequence
  Synthetic construct
LSGRSDDH

SEQ ID NO: 15 - protein, 13 aa, Artificial Sequence
  Synthetic construct
ISSGLLSGRSDNH

SEQ ID NO: 16 - protein, 13 aa, Artificial Sequence
  Synthetic construct
ISSGLLSGRSDQH

SEQ ID NO: 17 - protein, 13 aa, Artificial Sequence
  Synthetic construct
ISSGLLSGRSDDH

SEQ ID NO: 18 - protein, 8 aa, Artificial Sequence
  Synthetic construct
LSGRSDNH

SEQ ID NO: 19 - protein, 8 aa, Artificial Sequence
  Synthetic construct
LSGRSGNH

SEQ ID NO: 20 - protein, 12 aa, Artificial Sequence
  Synthetic construct
TSTSGRSANPRG

SEQ ID NO: 21 - protein, 8 aa, Artificial Sequence
  Synthetic construct
ISSGLLSS

SEQ ID NO: 22 - protein, 8 aa, Artificial Sequence
  Synthetic construct
QNQALRMA

SEQ ID NO: 23 - protein, 10 aa, Artificial Sequence
  Synthetic construct
VHMPLGFLGP

SEQ ID NO: 24 - protein, 8 aa, Artificial Sequence
  Synthetic construct
AVGLLAPP

SEQ ID NO: 25 - protein, 741 aa, Artificial Sequence
  Synthetic construct
GYGWGCEWNCGGSSPGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 8 aa, Artificial Sequence
  Synthetic construct
LSGRSDIH

SEQ ID NO: 27 - protein, 8 aa, Artificial Sequence
  Synthetic construct
LSGRSDQH

SEQ ID NO: 28 - protein, 8 aa, Artificial Sequence
  Synthetic construct
LSGRSDTH

SEQ ID NO: 29 - protein, 8 aa, Artificial Sequence
  Synthetic construct
LSGRSDYH

SEQ ID NO: 30 - protein, 8 aa, Artificial Sequence
  Synthetic construct
LSGRSDNP

SEQ ID NO: 31 - protein, 8 aa, Artificial Sequence
  Synthetic construct
LSGRSANP

SEQ ID NO: 32 - protein, 8 aa, Artificial Sequence
  Synthetic construct
LSGRSANI

SEQ ID NO: 33 - protein, 8 aa, Artificial Sequence
  Synthetic construct
LSGRSDNI

SEQ ID NO: 34 - protein, 13 aa, Artificial Sequence
  Synthetic construct
ISSGLLSGRSDNH

SEQ ID NO: 35 - protein, 13 aa, Artificial Sequence
  Synthetic construct
ISSGLLSGRSGNH

SEQ ID NO: 36 - protein, 15 aa, Artificial Sequence
  Synthetic construct
ISSGLLSGRSANPRG

SEQ ID NO: 37 - protein, 17 aa, Artificial Sequence
  Synthetic construct
AVGLLAPPSGRSANPRG

SEQ ID NO: 38 - protein, 13 aa, Artificial Sequence
  Synthetic construct
ISSGLLSGRSDDH

SEQ ID NO: 39 - protein, 13 aa, Artificial Sequence
  Synthetic construct
ISSGLLSGRSDIH

SEQ ID NO: 40 - skipped

SEQ ID NO: 41 - protein, 13 aa, Artificial Sequence
  Synthetic construct
ISSGLLSGRSDTH

SEQ ID NO: 42 - protein, 13 aa, Artificial Sequence
  Synthetic construct
ISSGLLSGRSDYH

SEQ ID NO: 43 - protein, 13 aa, Artificial Sequence
  Synthetic construct
ISSGLLSGRSDNP

SEQ ID NO: 44 - protein, 13 aa, Artificial Sequence
  Synthetic construct
ISSGLLSGRSANP

SEQ ID NO: 45 - protein, 13 aa, Artificial Sequence
  Synthetic construct
ISSGLLSGRSANI

SEQ ID NO: 46 - protein, 13 aa, Artificial Sequence
  Synthetic construct
ISSGLLSGRSDNI

SEQ ID NO: 47 - protein, 18 aa, Artificial Sequence
  Synthetic construct
AVGLLAPPGGLSGRSDNH

SEQ ID NO: 48 - protein, 18 aa, Artificial Sequence
  Synthetic construct
AVGLLAPPGGLSGRSDDH

SEQ ID NO: 49 - protein, 18 aa, Artificial Sequence
  Synthetic construct
AVGLLAPPGGLSGRSDIH

SEQ ID NO: 50 - protein, 18 aa, Artificial Sequence
  Synthetic construct
AVGLLAPPGGLSGRSDQH

SEQ ID NO: 51 - protein, 18 aa, Artificial Sequence
  Synthetic construct
AVGLLAPPGGLSGRSDTH

SEQ ID NO: 52 - protein, 18 aa, Artificial Sequence
  Synthetic construct
AVGLLAPPGGLSGRSDYH

SEQ ID NO: 53 - protein, 18 aa, Artificial Sequence
  Synthetic construct
AVGLLAPPGGLSGRSDNP

SEQ ID NO: 54 - protein, 18 aa, Artificial Sequence
  Synthetic construct
AVGLLAPPGGLSGRSANP

SEQ ID NO: 55 - protein, 18 aa, Artificial Sequence
  Synthetic construct
AVGLLAPPGGLSGRSANI

SEQ ID NO: 56 - protein, 18 aa, Artificial Sequence
  Synthetic construct
AVGLLAPPGGLSGRSDNI

SEQ ID NO: 57 - protein, 11 aa, Artificial Sequence
  Synthetic construct
RASQSIGTNIH

SEQ ID NO: 58 - protein, 7 aa, Artificial Sequence
  Synthetic construct
YASESIS

SEQ ID NO: 59 - protein, 9 aa, Artificial Sequence
  Synthetic construct
QQNNNWPTT

SEQ ID NO: 60 - protein, 5 aa, Artificial Sequence
  Synthetic construct
NYGVH

SEQ ID NO: 61 - protein, 16 aa, Artificial Sequence
  Synthetic construct
VIWSGGNTDYNTPFTS

SEQ ID NO: 62 - protein, 11 aa, Artificial Sequence
  Synthetic construct
ALTYYDYEFAY

SEQ ID NO: 63 - protein, 107 aa, Artificial Sequence
  Synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 64 - protein, 119 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSX

SEQ ID NO: 65 - protein, 214 aa, Artificial Sequence
  Synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 449 aa, Artificial Sequence
  Synthetic construct
  MISC_FEATURE (119)..(119): Xaa=Ser or Ala
  MISC_FEATURE (299)..(299): Xaa=any animo acid
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 449 aa, Artificial Sequence
  Synthetic construct
  MISC_FEATURE (119)..(119): Xaa=Ser or Ala
  MISC_FEATURE (236)..(236): Xaa= any amino acid
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEXLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 449 aa, Artificial Sequence
  Synthetic construct
  MISC_FEATURE (119)..(119): Xaa=Ser or Ala
  MISC_FEATURE (237)..(237): Xaa= any amino acid
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELXGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 449 aa, Artificial Sequence
  Synthetic construct
  MISC_FEATURE (119)..(119): Xaa=Ser or Ala
  MISC_FEATURE (333)..(333): Xaa= any amino acid
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAXIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 449 aa, Artificial Sequence
  Synthetic construct
  MISC_FEATURE (119)..(119): Xaa=Ser or Ala
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 450 aa, Artificial Sequence
  Synthetic construct
  MISC_FEATURE (120)..(120): Xaa=Ser or Ala
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSXASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 15 aa, Artificial Sequence
  Synthetic construct
LSCEGWAMNREQCRA

SEQ ID NO: 79 - protein, 15 aa, Artificial Sequence
  Synthetic construct
PPLECNTKSMCSKHD

SEQ ID NO: 80 - protein, 15 aa, Artificial Sequence
  Synthetic construct
DRDCRGRRARCQQEG

SEQ ID NO: 81 - protein, 15 aa, Artificial Sequence
  Synthetic construct
FTCEGWAMNREQCRT

SEQ ID NO: 82 - protein, 15 aa, Artificial Sequence
  Synthetic construct
GRCPPSRDIRFCTYM

SEQ ID NO: 83 - protein, 15 aa, Artificial Sequence
  Synthetic construct
FSCEGWAMNRSQCRT

SEQ ID NO: 84 - protein, 15 aa, Artificial Sequence
  Synthetic construct
FTCEGWAMNRDQCRT

SEQ ID NO: 85 - protein, 15 aa, Artificial Sequence
  Synthetic construct
CISPRGCPDGPYVMY

SEQ ID NO: 86 - protein, 14 aa, Artificial Sequence
  Synthetic construct
CISPRGCPDGPYVM

SEQ ID NO: 87 - protein, 7 aa, Artificial Sequence
  Synthetic construct
CISPRGC

SEQ ID NO: 88 - protein, 5 aa, Artificial Sequence
  Synthetic construct
GSGGS

SEQ ID NO: 89 - protein, 4 aa, Artificial Sequence
  Synthetic construct
GGGS

SEQ ID NO: 90 - protein, 4 aa, Artificial Sequence
  Synthetic construct
GGSG

SEQ ID NO: 91 - protein, 5 aa, Artificial Sequence
  Synthetic construct
GGSGG

SEQ ID NO: 92 - protein, 5 aa, Artificial Sequence
  Synthetic construct
GSGSG

SEQ ID NO: 93 - protein, 5 aa, Artificial Sequence
  Synthetic construct
GSGGG

SEQ ID NO: 94 - protein, 5 aa, Artificial Sequence
  Synthetic construct
GGGSG

SEQ ID NO: 95 - protein, 5 aa, Artificial Sequence
  Synthetic construct
GSSSG

SEQ ID NO: 96 - protein, 11 aa, Artificial Sequence
  Synthetic construct
GSSGGSGGSGG

SEQ ID NO: 97 - protein, 4 aa, Artificial Sequence
  Synthetic construct
GGGS

SEQ ID NO: 98 - protein, 15 aa, Artificial Sequence
  Synthetic construct
GGGGSGGGGSGGGGS

SEQ ID NO: 99 - protein, 5 aa, Artificial Sequence
  Synthetic construct
GGGGS

SEQ ID NO: 100 - protein, 13 aa, Artificial Sequence
  Synthetic construct
GSSGGSGGSGGSG

SEQ ID NO: 101 - protein, 23 aa, Artificial Sequence
  Synthetic construct
GSSGGSGGSGGGGGSGGGSGGGS

SEQ ID NO: 102 - protein, 23 aa, Artificial Sequence
  Synthetic construct
GSSGGSGGSGGSGGGSGGGSGGS

SEQ ID NO: 103 - protein, 5 aa, Artificial Sequence
  Synthetic construct
GSSGT

SEQ ID NO: 104 - protein, 8 aa, Artificial Sequence
  Synthetic construct
GGGSSGGS

SEQ ID NO: 105 - protein, 15 aa, Artificial Sequence
  Synthetic construct
MMYCGGNEIFCEPRG

SEQ ID NO: 106 - protein, 15 aa, Artificial Sequence
  Synthetic construct
GYGWGCEWNCGGSSP

SEQ ID NO: 107 - protein, 15 aa, Artificial Sequence
  Synthetic construct
MMYCGGNEIFCGPRG

SEQ ID NO: 108 - protein, 16 aa, Artificial Sequence
  Synthetic construct
GSTSGSGKPGSSEGST

SEQ ID NO: 109 - protein, 250 aa, Artificial Sequence
  Synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGSTSGSGKPGSSEGSTEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 110 - protein, 250 aa, Artificial Sequence
  Synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGSTSGSGKPGSSEGSTEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 111 - protein, 250 aa, Artificial Sequence
  Synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGSTSGSGKPGSSEGSTEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 112 - protein, 250 aa, Artificial Sequence
  Synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGSTSGSGKPGSSEGSTEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 113 - protein, 330 aa, Artificial Sequence
  Synthetic construct
  MISC_FEATURE (180)..(180): Xaa=any amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 330 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 330 aa, Artificial Sequence
  Synthetic construct
  MISC_FEATURE (117)..(117): Xaa=any amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEXLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 449 aa, Artificial Sequence; identical to SEQ ID NO: 70
  Synthetic construct
  MISC_FEATURE (119)..(119): Xaa=Ser or Ala

SEQ ID NO: 117 - protein, 449 aa, Artificial Sequence; identical to SEQ ID NO: 71
  Synthetic construct
  MISC_FEATURE (119)..(119): Xaa=Ser or Ala
  MISC_FEATURE (237)..(237): Xaa=any amino acid

SEQ ID NO: 118 - protein, 330 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 330 aa, Artificial Sequence
  Synthetic construct
  MISC_FEATURE (214)..(214): Xaa=any amino acid
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAXIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 330 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 330 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 330 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - DNA, 2292 bp, Artificial Sequence
  Synthetic construct
gctcagctcctggggctcctgctactatggctccgcggtgctagatgtcaaggccagtctggatccggttatctgtggggttgcgagtggaattgcggagggatcactacaggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggctccacctccggctccggcaagcccggctcctccgagggctccaccgaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 124 - protein, 742 aa, Artificial Sequence
  Synthetic construct
GYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGSTSGSGKPGSSEGSTEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 2289 bp, Artificial Sequence
  Synthetic construct
gctcagctcctggggctcctgctactatggctccgcggtgctagatgtcaaggccagtctggatccatgatgtactgcggcggaaacgagatcttctgcgagcccagaggcggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 126 - protein, 741 aa, Artificial Sequence
  Synthetic construct
MMYCGGNEIFCEPRGGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - DNA, 2289 bp, Artificial Sequence
  Synthetic construct
gctcagctcctggggctcctgctactatggctccgcggtgctagatgtcaaggccagtctggatccatgatgtactgcggcggaaacgagatcttctgcggccctagaggcggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 128 - protein, 741 aa, Artificial Sequence
  Synthetic construct
MMYCGGNEIFCGPRGGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - DNA, 2241 bp, Artificial Sequence
  Synthetic construct
caaggccagtctggatccggttatctgtggggttgcgagtggaattgcggagggatcactacaggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 130 - protein, 747 aa, Artificial Sequence
  Synthetic construct
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - DNA, 783 bp, Artificial Sequence
  Synthetic construct
caaggccagtctggccaaggtcttagttgtgaaggttgggcgatgaatagagaacaatgtcgagccggaggtggctcgagcggcggctctatctcttccggactgctgtccggcagatccgaccagcacggcggaggatcccaaatcctgctgacacagtctcctgtcatactgagtgtctcccccggcgagagagtctctttctcatgtcgggccagtcagtctattgggactaacatacactggtaccagcaacgcaccaacggaagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 132 - protein, 261 aa, Artificial Sequence
  Synthetic construct
QGQSGQGLSCEGWAMNREQCRAGGGSSGGSISSGLLSGRSDQHGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 6 aa, Artificial Sequence
  Synthetic construct
QGQSGS

SEQ ID NO: 134 - DNA, 18 bp, Artificial Sequence
  Synthetic construct
caaggccagtctggatcc

SEQ ID NO: 135 - DNA, 2223 bp, Artificial Sequence
  Synthetic construct
ggttatctgtggggttgcgagtggaattgcggagggatcactacaggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 136 - protein, 741 aa, Artificial Sequence
  Synthetic construct
GYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - DNA, 21 bp, Artificial Sequence
  Synthetic construct
caaggccagtctggccaaggt

SEQ ID NO: 138 - protein, 7 aa, Artificial Sequence
  Synthetic construct
QGQSGQG

SEQ ID NO: 139 - DNA, 762 bp, Artificial Sequence
  Synthetic construct
cttagttgtgaaggttgggcgatgaatagagaacaatgtcgagccggaggtggctcgagcggcggctctatctcttccggactgctgtccggcagatccgaccagcacggcggaggatcccaaatcctgctgacacagtctcctgtcatactgagtgtctcccccggcgagagagtctctttctcatgtcgggccagtcagtctattgggactaacatacactggtaccagcaacgcaccaacggaagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 140 - protein, 254 aa, Artificial Sequence
  Synthetic construct
LSCEGWAMNREQCRAGGGSSGGSISSGLLSGRSDQHGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - DNA, 18 bp, Artificial Sequence
  Synthetic construct
atggacatgagggtcccc

SEQ ID NO: 142 - DNA, 2289 bp, Artificial Sequence
  Synthetic construct
gctcagctcctggggctcctgctactatggctccgcggtgctagatgtcaaggccagtctggatccggttatctgtggggttgcgagtggaattgcggagggtcctcccccggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 143 - protein, 249 aa, Artificial Sequence
  Synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 144 - protein, 249 aa, Artificial Sequence
  Synthetic construct
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 145 - protein, 249 aa, Artificial Sequence
  Synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 146-148 - skipped (3 entries)

SEQ ID NO: 149 - protein, 14 aa, Artificial Sequence
  Synthetic construct
RSSTGAVTTSNYAN

SEQ ID NO: 150 - protein, 249 aa, Artificial Sequence
  Synthetic construct
QAVVTQEPSLTVSPGGTVTLTCRSSTGAVTTSNYANWVQQKPGQAPRGLIGGTNKRAPGTPARFSGSLIGGKAALTLSGAQPEDEAEYYCALWYSNLWVFGGGTKLTVLGGGGSGGGGSGGGGSEVQLVESGGGLVQPGGSLRLSCAASGFTFSTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 151-153 - skipped (3 entries)

SEQ ID NO: 154 - DNA, 2310 bp, Artificial Sequence
  Synthetic construct
atggacatgagggtccccgctcagctcctggggctcctgctactatggctccgcggtgctagatgtcaaggccagtctggatccggttatctgtggggttgcgagtggaattgcggagggatcactacaggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggctccacctccggctccggcaagcccggctcctccgagggctccaccgaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 155 - protein, 748 aa, Artificial Sequence
  Synthetic construct
QGQSGSGYLWGCEWNCGGITTGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGSTSGSGKPGSSEGSTEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - DNA, 2307 bp, Artificial Sequence
  Synthetic construct
atggacatgagggtccccgctcagctcctggggctcctgctactatggctccgcggtgctagatgtcaaggccagtctggatccatgatgtactgcggcggaaacgagatcttctgcgagcccagaggcggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 157 - protein, 747 aa, Artificial Sequence
  Synthetic construct
QGQSGSMMYCGGNEIFCEPRGGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - DNA, 2307 bp, Artificial Sequence
  Synthetic construct
atggacatgagggtccccgctcagctcctggggctcctgctactatggctccgcggtgctagatgtcaaggccagtctggatccggttatctgtggggttgcgagtggaattgcggagggtcctcccccggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 159 - protein, 747 aa, Artificial Sequence
  Synthetic construct
QGQSGSGYGWGCEWNCGGSSPGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 2307 bp, Artificial Sequence
  Synthetic construct
atggacatgagggtccccgctcagctcctggggctcctgctactatggctccgcggtgctagatgtcaaggccagtctggatccatgatgtactgcggcggaaacgagatcttctgcggccctagaggcggctcgagcggtggcagcggtggctctggtggtctgagcggccgttccgatgatcatggcggcggttctcaaactgtagtaactcaagaaccaagcttctccgtctcccctgggggaacagtcacacttacctgccgaagtagtacaggtgctgttacgaccagtaactatgccaattgggtacaacaaacgcctggtcaggctccgcgcggattgataggaggcacgaataaacgggcacccggtgtcccggacagattcagcggaagcatactcggtaataaggcagctcttactatcactggggcccaagctgatgatgaaagtgattattattgtgcgctctggtacagcaacctctgggtgtttgggggtggcacgaaacttactgtcttgggcggcggcggatcagggggaggtggctctggaggaggaggctcagaagtccaactggtcgaatccgggggagggctcgtacagccgggtgggtccctcaaactctcttgtgcggcctcagggtttaccttcagtacatacgcgatgaattgggtccggcaggccagtgggaaagggctcgaatgggtaggacgaatccgatcaaaatacaacaactacgctacttattacgctgattccgtgaaggacagattcacaatatcccgcgacgatagcaagaatacggcatatcttcagatgaattctcttaaaactgaggataccgctgtgtattactgcacaagacatggtaattttggaaactcatatgtctcttggttcgcttattggggacagggcacgttggttaccgtgtctagcggaggtggtggatcccaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaatttgaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcctcaatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 161 - protein, 747 aa, Artificial Sequence
  Synthetic construct
QGQSGSMMYCGGNEIFCGPRGGSSGGSGGSGGLSGRSDDHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0324088 A1
(43) Pub. Date: Oct. 21, 2021

(54) ANTIBODIES, ACTIVATABLE ANTIBODIES, BISPECIFIC ANTIBODIES, AND BISPECIFIC ACTIVATABLE ANTIBODIES AND METHODS OF USE THEREOF

(71) Applicant: CYTOMX THERAPEUTICS, INC., South San Francisco, CA (US)

(72) Inventors: Madan M. PAIDHUNGAT, South San Francisco, CA (US); Ellaine Anne Mariano FOX, South San Francisco, CA (US); Li MEI, South San Francisco, CA (US)

(73) Assignee: CYTOMX THERAPEUTICS, INC., South San Francisco, CA (US)

(21) Appl. No.: 17/052,088

(22) PCT Filed: May 2, 2019
(86) PCT No.: PCT/US2019/030472

§ 371 Date: Oct. 30, 2020

Related U.S. Application Data
(60) Provisional application No. 62/666,056, filed on May 2, 2018.
(60) Provisional application No. 62/712,046, filed on Jul. 30, 2018.

(51) Int. Cl. C07K 16/28; A61K 39/395; A61P 35/00

(52) U.S. Cl. CPC C07K 16/2863; C07K 16/2809; A61K 39/3955; A61P 35/00; C07K 2317/515; C07K 2317/76; C07K 2317/622; C07K 2317/31; C07K 2317/52; C07K 2319/50; C07K 2317/92; C07K 2317/51

(57) ABSTRACT

Provided herein antibodies, activatable antibodies (AAs), bispecific antibodies, and bispecific activatable antibodies (BAAs). Also provided herein are methods of making and methods of use of these antibodies, AAs, bispecific antibodies, and BAAs.

### CROSS-REFERENCE TO RELATED APPLICATIONS

This application claims the benefit of U.S. Provisional Application No. 62/666,056, filed on May 2, 2018, No. 62/712,046, filed on Jul. 30, 2018, pursuant 35 U.S.C. § 119(e), each of which is incorporated herein by reference in its entirety.

### REFERENCE TO SEQUENCE LISTING

The Sequence Listing submitted electronically concurrently herewith pursuant 37 C.F.R. § 1.821 in computer readable form (ASCII format) via EFS-Web as file name CYTM_060_001 WO_SeqList_ST25.txt is incorporated herein by reference. The ASCII copy of the Sequence Listing was created on May 1, 2019 and is 224 kilobytes in size.

### BACKGROUND

Antibody-based therapies have proven effective treatments for several diseases but in some cases, toxicities due to broad target expression have limited their therapeutic effectiveness. In addition, antibody-based therapeutics have exhibited other limitations such as rapid clearance from the circulation following administration.

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

Accordingly, there is a continued need in the field of antibody-based therapeutics for antibodies that mimic the desirable characteristics of the small molecule prodrug.

### SUMMARY

Provided herein are antibodies, bispecific antibodies, activatable antibodies, and bispecific activatable antibodies, methods of making, and methods of use thereof. These find use in therapeutics and diagnostics. The activatable antibodies and bispecific activatable antibodies of the present disclosure may be used to reduce damage to healthy tissue generally caused by an antibody binding to its target on healthy tissue as well as on diseased tissue.

In one aspect, provided herein is an activatable antibody (AA) comprising:

(a) at least one scFv comprising a light chain variable region (VL) linked to a heavy chain variable region (VH), wherein the VL is linked to the VH by a linker comprising amino acid sequence SEQ ID NO: 108; and
(b) a prodomain comprising:

(i) a masking moiety (MM) coupled to the scFv, wherein the MM reduces or inhibits the binding of the scFV to its target when the AA is in an uncleaved state; and

(ii) a cleavable moiety (CM) coupled to the scFv, wherein the CM is a polypeptide that functions as a substrate for a protease.

In some embodiments, the AB binds to CD3ε.

In another aspect, provided herein is an activatable antibody (AA) comprising:

(a) an antibody or antigen binding fragment thereof (collectively referred to as an AB throughout) that specifically binds to CD3ε; and
(b) a prodomain comprising:

(i) a masking moiety (MM) coupled to the AB, wherein the MM reduces or inhibits the binding of the AB to the CD3ε when the AA is in an uncleaved state, wherein the MM comprises the amino acid sequence SEQ ID NO: 105 or SEQ ID NO: 106 or SEQ ID NO: 107; and

(ii) a cleavable moiety (CM) coupled to the AB, wherein the CM is a polypeptide that functions as a substrate for a protease.

In another aspect, provided herein is a bispecific activatable antibody (BAA), wherein said BAA, when activated, specifically binds to two targets, and wherein said BAA, when not activated, comprises the following structure:

a) an IgG antibody (AB1) that specifically binds to a first target wherein the AB1 comprises:

(i) two heavy chains (AB1 HCs) and two light chains (AB1 LCs); and

(ii) two first prodomains, each comprising a first masking moiety (MM1) linked to a first cleavable moiety (CM1) in the N-terminal to C-terminal direction, wherein the carboxyl terminus of each first prodomain is linked to the amino terminus of each light chain of the AB1, wherein
the MM1 reduces the binding of the AB1 to its target; and
the CM1 is a polypeptide that functions as a substrate for a first protease,
   b) two scFvs (AB2) that each specifically binds CD3ε, wherein each AB2 comprises:

(i) a light chain variable region (VL) linked to heavy chain variable region (VH), wherein the carboxyl terminus of each AB2 is linked to the amino terminus of each of the AB1 heavy chains, wherein
the VL comprises an amino acid sequence as set forth in SEQ ID NO: 1 or SEQ ID NO: 4 and the VH comprises an amino acid sequence as set forth in SEQ ID NO: 2 or SEQ ID NO: 3; and
the VL is linked to the VH by a linker L3 comprising an amino acid sequence selected from the group consisting of SEQ ID NO: 98 and SEQ ID NO: 108; and

(ii) two second prodomains, each comprising a second masking moiety (MM2) linked to a second cleavable moiety (CM2), in the N-terminal to C-terminal direction, wherein the carboxyl terminus of each second prodomain is linked to the amino terminus of each AB2 wherein
the MM2 reduces the binding of the AB2 to CD3ε;
the MM2 comprises an amino acid sequence selected from the group consisting of SEQ ID NO: 12, SEQ ID NO: 105, SEQ ID NO: 106 and SEQ ID NO: 107; and
the CM2 is a polypeptide that functions as a substrate for a second protease.

In another aspect, provided herein are pharmaceutical compositions comprising the AAs or BAAs, and, optionally, a carrier.

In another aspect provided herein is a method of treating, alleviating a symptom of, or delaying the progression of a disorder or disease (e.g. cancer) comprising administering a therapeutically effective amount of the AAs or BAAs or of a pharmaceutical composition comprising an effective amount of the AAs or BAAs.

In another aspect, provided herein is any of the AAs, BAAs, or pharmaceutical compositions of the present disclosure for use as a medicament. In a related aspect, provided herein is an AA, a BAA, or a pharmaceutical composition of the present disclosure for use in a method of treatment, optionally wherein the method is for the treatment or prevention of a neoplasm, a cancer, or a tumor. In some embodiments, the cancer may be bladder cancer, breast cancer, cervical cancer, cholangiocarcinoma, colorectal cancer, endometrial cancer, esophageal cancer, gastric cancer, glioblastoma, head and neck cancer, liver cancer, lung cancer, ovarian cancer, pancreatic cancer, prostate cancer, renal cancer, sarcoma, squamous cell cancer, or skin cancer. In some embodiments, the cancer may be associated with cells expressing EGFR, for instance the tumor cells may comprise cells expressing EGFR or the tumor microenvironment may comprise cells expressing EGFR. In some embodiments, provided herein is an AA, a BAA, or a pharmaceutical composition of the present disclosure for use in a method of treating a disorder or disease comprising disease cells expressing EGFR. In some embodiments, provided herein is an AA, a BAA, or a pharmaceutical composition of the present disclosure for use in a method of treatment, wherein the treatment comprises the inhibition of angiogenesis.

In another aspect, provided herein are methods of making the AAs and BAAs.

### BRIEF DESCRIPTION OF THE DRAWINGS

FIG. 1 demonstrates concentration-dependent aggregation of the dually masked bispecific activatable antibodies of Table 11, as assessed by studying the relation between monomer content and concentration.

FIG. 2A demonstrates binding of intact (not activated) and activated BAAs CI138, CI139, and CI140 compared to CI106 as assessed by CD3ε-based ELISA.

FIG. 2B demonstrates binding of intact and activated BAAs CI138, CI139, and CI140 compared to CI106, as assessed by EGFR-based ELISA.

FIG. 3 demonstrates biological activity of intact and activated BAAs CI138, CI139, and CI140 compared to CI106, as assessed by cytotoxicity assays.

FIG. 4 illustrates an exemplary BAA provided herein.

FIG. 5, which plots tumor volume versus days post initial treatment dose, demonstrates a dose-dependent effect of CI106 and CI139 dually masked, bispecific, activatable antibodies on the growth of HT29-luc2 xenograft tumors. The most efficacious dose tested was 3.0 mg/kg, which led to tumor regression.

FIG. 6, demonstrates the binding of CI106 and CI139 to HT29 and Jurkat cells via a flow cytometry-based binding assay compared to the activated bispecific antibody

FIG. 7 demonstrates biological activity of intact and activated BAA CI139 compared to CI106, as assessed by cytotoxicity assays in different cell lines.

FIG. 8, which plots tumor volume versus days post initial treatment dose, demonstrates a dose-dependent effect of CI106 and CI139 dually masked, bispecific, activatable antibodies on the growth of HCT116 xenograft tumors.

FIG. 9 demonstrates the pharmacokinetics in cynomolgus monkey of the dually masked molecules CI106 and CI139.

### DETAILED DESCRIPTION OF THE INVENTION

Provided herein are antibodies, activatable antibodies (AAs), bispecific antibodies, and bispecific activatable antibodies (BAAs).

In some embodiments, provided herein are humanized antibodies that specifically bind to the epsilon chain of CD3 (CD3ε; referred to herein interchangeably as CD3). In some embodiments, provided herein are scFv antibodies that specifically bind to CD3. In some embodiments, provided herein are IgG antibodies that specifically bind to CD3. In some embodiments, the anti-CD3 antibodies (e.g., anti-CD3 scFv or IgG, such as IgG1, antibodies) comprise point mutations in the Fc region, such that the antibody has reduced effector function

In some embodiments, provided herein are IgG antibodies that specifically bind to Epidermal Growth Factor Receptor (EGFR). In some embodiments, provided herein are IgG1 antibodies that specifically bind to EGFR, wherein the antibodies comprise point mutations in the Fc region, such that the antibody has reduced effector function.

In some embodiments provided herein are AAs, for example AAs that specifically bind to EGFR or CD3. These AAs are optimized for affinity, effector function, masking, and cleavability.

In some embodiments, provided herein are BAAs, for example BAAs that bind to a target antigen (e.g. tumor antigen, such as a target presented in Table 9) and a second antigen (e.g. immune effector antigen on an immune effector cell). In some embodiments, the immune effector cell is a leukocyte cell. In some embodiments, the immune effector cell is a T cell. In some embodiments, the immune effector cell is a natural killer (NK) cell. In some embodiments, the immune effector cell is a macrophage. In some embodiments, the immune effector cell is a mononuclear cell, such as a myeloid mononuclear cell. In some embodiments, the BAAs are immune effector cell-engaging BAAs. In some embodiments, the BAAs are leukocyte cell-engaging BAAs. In some embodiments, the BAAs are T cell engaging bispecific (TCB) AAs. In some embodiments, the BAAs are NK cell-engaging BAAs. In some embodiments, the BAAs are macrophage cell-engaging BAAs. In some embodiments, the BAAs are mononuclear cell-engaging BAAs, such as myeloid mononuclear cell-engaging BAAs. In some embodiments, the BAAs bind EGFR and CD3. These BAAs are optimized for affinity, effector function, masking, and cleavability.

Also provided herein are methods of making and methods of use of these antibodies, AAs, and BAAs. AAs, including general production thereof and identification of masking moieties (MMs) and cleavable moieties (CMs) is described in International Publication Numbers WO 2009/025846 by Daugherty et al., published 26 Feb. 2009, and WO 2010/081173 by Stagliano et al., published 15 Jul. 2010, both of which are incorporated by reference in their entirety. BAAs, including general production thereof and identification of masking moieties (MMs) and cleavable moieties (CMs) is described in International Publication Numbers WO2015/013671 by Lowman et al., published 29 Jan. 2015 and WO2016/014974 by Irving et al., published 28 Jan. 2016, both of which are incorporated by reference in their entirety. Also incorporated by reference are International Publication WO2016/014974 by Irving et al., published 28 Jan. 2016, and International Publication WO2016/118629 by Moore et al., published 28 Jul. 2016 which provide AAs, general production, MMs, and CMs.

As used herein, unless specified otherwise, the term “antibody” includes an antibody or antigen-binding fragment thereof that specifically binds its target and is a monoclonal antibody, domain antibody, single chain, Fab fragment, a F(ab′)2 fragment, a scFv, a scAb, a dAb, a single domain heavy chain antibody, and a single domain light chain antibody. In some embodiments, the antibody is an IgG antibody. In some embodiments, the antibody is an IgG1 antibody. In some embodiments, the antibody is an IgG4 antibody. In some embodiments, the antibody is a scFv antibody. In some embodiments, such an antibody or immunologically active fragment thereof that binds its target is a mouse, chimeric, humanized or fully human monoclonal antibody.

### 1. CD3 Antibodies

Provided herein is an antibody or antigen binding fragment thereof (AB) that specifically binds to the epsilon chain of CD3 (CD3ε, referred to herein throughout as CD3). Provided herein are activatable antibodies (AAs) and bispecific activatable antibodies (BAAs) comprising these CD3 ABs.

Exemplary amino acid sequences of CD3-binding antibodies of the disclosure (variable domains) are provided in Table 1. (Predicted CDR sequences are underlined). As provided below, L3 is a linker, linking the light and heavy chain variable domains, in the exemplary CD3-binding antibodies.

TABLE 1
Anti-CD3 variant v12
Light Chain Variable Domain LV12
QTVVTQEPSFSVSPGGTVTLTCRSSTGAVTTSNYANWVQQTPGQAPRGLI
GGTNKRAPGVPDRFSGSILGNKAALTITGAQADDESDYYCALWYSNLWVF
GGGTKLTVL (SEQ ID NO: 1)
Heavy Chain Variable Domain HV12
EVQLVESGGGLVQPGGSLRLSCAASGFTFSTYAMNWVRQAPGKGLEWVA
RIRSKYNNYATYYADSVKDRFTISRDDSKNSLYLQMNSLKTEDTAVYYC
VRHGNFGNSYVSWFAYWGQGTLVTVSS (SEQ ID NO: 2)
LV12-L3-HV12, wherein L3 is SEQ ID NO: 98
QTVVTQEPSFSVSPGGTVTLTCRSSTGAVTTSNYANWVQQTPGQAPRGLI
GGTNKRAPGVPDRFSGSILGNKAALTITGAQADDESDYYCALWYSNLWVF
GGGTKLTVL[GGGGSGGGGSGGGGS]EVQLVESGGGLVQPGGSLRLSCAA
SGFTFSTYAMNWVRQAPGKGLEWVARIRSKYNNYATYYADSVKDRFTISR
DDSKNSLYLQMNSLKTEDTAVYYCVRHGNFGNSYVSWFAYWGQGTLVTVS
S (SEQ ID NO: 143)
LV12-L3-HV12, wherein L3 is SEQ ID NO: 108
QTVVTQEPSFSVSPGGTVTLTCRSSTGAVTTSNYANWVQQTPGQAPRGLI
GGTNKRAPGVPDRFSGSILGNKAALTITGAQADDESDYYCALWYSNLWVF
GGGTKLTVL[GSTSGSGKPGSSEGST]EVQLVESGGGLVQPGGSLRLSCA
ASGFTFSTYAMNWVRQAPGKGLEWVARIRSKYNNYATYYADSVKDRFTIS
RDDSKNSLYLQMNSLKTEDTAVYYCVRHGNFGNSYVSWFAYWGQGTLVTV
SS (SEQ ID NO: 109)
Anti-CD3 variant v16
Light Chain Variable Domain LV12
Sequence provided above
Heavy Chain Variable Domain HV20
EVQLVESGGGLVQPGGSLKLSCAASGFTFSTYAMNWVRQASGKGLEWVG
RIRSKYNNYATYYADSVKDRFTISRDDSKNTAYLQMNSLKTEDTAVYYC
TRHGNFGNSYVSWFAYWGQGTLVTVSS (SEQ ID NO: 3)
LV12-L3-HV20 wherein L3 is SEQ ID NO: 98
QTVVTQEPSFSVSPGGTVTLTCRSSTGAVTTSNYANWVQQTPGQAPRGLI
GGTNKRAPGVPDRFSGSILGNKAALTITGAQADDESDYYCALWYSNLWVF
GGGTKLTVL[GGGGSGGGGSGGGGS]EVQLVESGGGLVQPGGSLKLSCAA
SGFTFSTYAMNWVRQASGKGLEWVGRIRSKYNNYATYYADSVKDRFTISR
DDSKNTAYLQMNSLKTEDTAVYYCTRHGNFGNSYVSWFAYWGQGTLVTVS
S (SEQ ID NO: 144)
LV12-L3-HV20 wherein L3 is SEQ ID NO: 108
QTVVTQEPSFSVSPGGTVTLTCRSSTGAVTTSNYANWVQQTPGQAPRGLI
GGTNKRAPGVPDRFSGSILGNKAALTITGAQADDESDYYCALWYSNLWVF
GGGTKLTVL[GSTSGSGKPGSSEGST]EVQLVESGGGLVQPGGSLKLSCA
ASGFTFSTYAMNWVRQASGKGLEWVGRIRSKYNNYATYYADSVKDRFTIS
RDDSKNTAYLQMNSLKTEDTAVYYCTRHGNFGNSYVSWFAYWGQGTLVTV
SS (SEQ ID NO: 110)
Anti-CD3 variant v19
Light Chain Variable Domain LV19
QAVVTQEPSLTVSPGGTVTLTCRSSTGAVTTSNYANWVQQKPGQAPRGLI
GGTNKRAPGTPARFSGSLIGGKAALTLSGAQPEDEAEYYCALWYSNLWVF
GGGTKLTVL (SEQ ID NO: 4)
Heavy Chain Variable Domain HV20
Sequence provided above
LV19-L3-HV20 wherein L3 is SEQ ID NO: 98
QAVVTQEPSLTVSPGGTVTLTCRSSTGAVTTSNYANWVQQKPGQAPRGLI
GGTNKRAPGTPARFSGSLIGGKAALTLSGAQPEDEAEYYCALWYSNLWVF
GGGTKLTVL[GGGGSGGGGSGGGGS]EVQLVESGGGLVQPGGSLKLSCAA
SGFTFSTYAMNWVRQASGKGLEWVGRIRSKYNNYATYYADSVKDRFTISR
DDSKNTAYLQMNSLKTEDTAVYYCTRHGNFGNSYVSWFAYWGQGTLVTVS
S (SEQ ID NO: 145)
LV19-L3-HV20 wherein L3 is SEQ ID NO: 108
QAVVTQEPSLTVSPGGTVTLTCRSSTGAVTTSNYANWVQQKPGQAPRGLI
GGTNKRAPGTPARFSGSLIGGKAALTLSGAQPEDEAEYYCALWYSNLWVF
GGGTKLTVL[GSTSGSGKPGSSEGST]EVQLVESGGGLVQPGGSLKLSCA
ASGFTFSTYAMNWVRQASGKGLEWVGRIRSKYNNYATYYADSVKDRFTIS
RDDSKNTAYLQMNSLKTEDTAVYYCTRHGNFGNSYVSWFAYWGQGTLVTV
SS (SEQ ID NO: 111)
Anti-CD3 variant v26
Light Chain Variable Domain LV19
Sequence provided above
Heavy Chain Variable Domain HV12
Sequence provided above
LV19-L3-HV12, wherein L3 is SEQ ID NO: 98
QAVVTQEPSLTVSPGGTVTLTCRSSTGAVTTSNYANWVQQKPGQAPRGLI
GGTNKRAPGTPARFSGSLIGGKAALTLSGAQPEDEAEYYCALWYSNLWVF
GGGTKLTVL[GGGGSGGGGSGGGGS]EVQLVESGGGLVQPGGSLRLSCAA
SGFTFSTYAMNWVRQAPGKGLEWVARIRSKYNNYATYYADSVKDRFTISR
DDSKNSLYLQMNSLKTEDTAVYYCVRHGNFGNSYVSWFAYWGQGTLVTVS
S (SEQ ID NO: 150)
LV19-L3-HV12, wherein L3 is SEQ ID NO: 108
QAVVTQEPSLTVSPGGTVTLTCRSSTGAVTTSNYANWVQQKPGQAPRGLI
GGTNKRAPGTPARFSGSLIGGKAALTLSGAQPEDEAEYYCALWYSNLWVF
GGGTKLTVL[GSTSGSGKPGSSEGST]EVQLVESGGGLVQPGGSLRLSCA
ASGFTFSTYAMNWVRQAPGKGLEWVARIRSKYNNYATYYADSVKDRFTIS
RDDSKNSLYLQMNSLKTEDTAVYYCVRHGNFGNSYVSWFAYWGQGTLVTV
SS (SEQ ID NO: 112)

Exemplary scFv linkers (referred to herein as “L3” linking a VL and VH) are provided in Table 1-1.

TABLE 1-1

| SEQ ID NO: | Linker Amino Acid Sequence |
|---|---|
| 98 | GGGGSGGGGSGGGGS |
| 108 | GSTSGSGKPGSSEGST |

Exemplary CDR sequences of CD3-binding antibodies are provided in Table 2.

TABLE 2

| Name | CD3 Ab CDR Sequences | SEQ ID NO: |
|---|---|---|
| SP34L1 | RSSTGAVTTSNYAN | SEQ ID NO: 149 |
| SP34L2 | GTNKRAP | SEQ ID NO: 5 |
| SP34L3 | ALWYSNLWV | SEQ ID NO: 6 |
| SP34H1 | TYAMN | SEQ ID NO: 7 |
| SP34H2 | RIRSKYNNYATYYADSVKD | SEQ ID NO: 8 |
| SP34H3 | HGNFGNSYVSWFAY | SEQ ID NO: 9 |

As provided herein, the CD3 antibody comprises at least one, at least two, at least three, at least four, or at least five of the CDR sequences provided in Table 2. In some embodiments, the CD3 antibody comprises the six CDR sequences provided in Table 2.

In some embodiments, the CD3 antibody comprises heavy chain variable domain as set forth in SEQ ID NO: 2.

In some embodiments, the CD3 antibody comprises a heavy chain variable domain as set forth in SEQ ID NO: 3.

In some embodiments, the CD3 antibody comprises a light chain variable domain as set forth in SEQ ID NO: 1.

In some embodiments, the CD3 antibody comprises a light chain variable domain as set forth in SEQ ID NO: 4.

In some embodiments, the CD3 antibody comprises a heavy chain variable domain as set forth in SEQ ID NO: 2 and a light chain variable domain as set forth in SEQ ID NO: 1.

In some embodiments, the CD3 antibody comprises a heavy chain variable domain as set forth in SEQ ID NO: 3 and a light chain variable domain as set forth in SEQ ID NO: 1.

In some embodiments, the CD3 antibody comprises a heavy chain variable domain as set forth in SEQ ID NO: 3 and a light chain variable domain as set forth in SEQ ID NO: 4.

In some embodiments, the CD3 antibody comprises a heavy chain variable domain as set forth in SEQ ID NO: 2 and a light chain variable domain as set forth in SEQ ID NO: 4.

In some embodiments, the CD3 antibody comprises a heavy chain variable domain as set forth in SEQ ID NO: 2 or SEQ ID NO: 3 or comprises a light chain variable domain as set forth in SEQ ID NO: 1 or SEQ ID NO: 4.

In some embodiments, the CD3 antibody comprises a heavy chain variable domain as set forth in SEQ ID NO: 2 or SEQ ID NO: 3 and comprises a light chain variable domain as set forth in SEQ ID NO: 1 or SEQ ID NO: 4.

In some embodiments, the antibody comprises a sequence 70%, 80%, 90%, 95%, 99%, or 100% similar to a heavy chain variable domain as set out above, and/or comprises a sequence 70%, 80%, 90%, 95%, 99%, or 100% similar to a light chain variable domain as set out above, and specifically binds to CD3. In some embodiments, heavy chain variable domain is in combination with a light chain variable domain as set out above. In some embodiments, the antibody comprises a sequence 70%, 80%, 90%, 95%, 99%, or 100% similar to a heavy chain variable domain as set out above, and/or comprises a sequence 70%, 80%, 90%, 95%, 99%, or 100% similar to a light chain variable domain as set out above, comprises the six CDR sequences provided in Table 2, and specifically binds to CD3.

In some embodiments, the CD3 antibody is a scFv antibody. In some embodiments, the variable domains comprise the following structure from N terminus to C terminus: LV-HV. In some embodiments, the variable domains comprise the following structure from N terminus to C terminus: HV-LV.

In some embodiments, the CD3 antibody is a scFv antibody comprising a light chain variable region (VL) linked to a heavy chain variable region (VH), wherein the VL is linked to the VH by a linker comprising amino acid sequence SEQ ID NO: 98. Exemplary sequences with such a linker are provided in Table 1.

In some embodiments, the CD3 antibody is a scFv antibody comprising a light chain variable region (VL) linked to a heavy chain variable region (VH), wherein the VL is linked to the VH by a linker comprising amino acid sequence SEQ ID NO: 108. Exemplary sequences with such a linker are provided in Table 1.

In exemplary embodiments, provided herein is an antibody that specifically binds to CD3 (AB), wherein the antibody is an IgG1 antibody or a scFv linked to an Fc domain, wherein the antibody comprises an Fc region comprising an amino acid substitution in at least one of amino acid positions L234, L235, and P331, as numbered by the EU index as set forth in Kabat, such that the antibody has reduced effector function. In some embodiments, the amino acid substitution is any one or more of L234F, L235E, and P331S. In some embodiments, the antibody comprises amino acid substitutions in at least two of amino acid positions L234, L235, and P331. In some embodiments, the antibody comprises amino acid substitutions at amino acid positions L234, L235, and P331. In some embodiments, the antibody comprises L234F, L235E, and P331S amino acid substitutions. In some embodiments, the antibody comprises an Fc region comprising an amino acid substitution at N297. In some embodiments, the Fc region comprises an N297Q mutation. In some embodiments, the antibody comprises L234F, L235E, P331S, and N297Q amino acid substitutions. In some embodiments, the heavy chain variable domain of the antibody with reduced effector function comprises any one of SEQ ID NO: 2 or SEQ ID NO: 3 or wherein the light chain variable domain of the AB comprises any one of SEQ ID NO: 1 or SEQ ID NO: 4.

### 2. Activatable CD3 Antibodies

In some embodiments, any one of the CD3 antibodies provided herein is in an activatable antibody (AA) format.

As generally provided herein, the AAs of the invention comprise MM-CM constructs, also referred to herein as a prodomain. Accordingly, as used herein, the term “prodomain” refers to a polypeptide comprising a masking moiety (MM) and a cleavable moiety (CM). In some embodiments, the MM and the CM are separated by a linker, referred to herein as L1, In some embodiments, the prodomain comprises a linker at the carboxyl terminus of the CM; this linker, referred to herein as L2, links the CM of the prodomain to the AB. In some embodiments, the prodomain comprises a linker between MM and CM and a linker after CM. In some embodiments, the MM and the CM are not separated by a linker. In certain embodiments a prodomain comprises one of the following formulae (where the formula below represents an amino acid sequence in either N- to C-terminal direction or C- to N-terminal direction): (MM)-L1-(CM), (MM)-(CM)-L2, (MM)-L1-(CM)-L2, or (MM)-(CM). In exemplary embodiments, a prodomain comprises an EGFR MM and a CM cleavable by a matriptase or MMP; or a CD3ε MM and a CM cleavable by a matriptase or MMP. In some embodiments, a prodomain comprises an EGFR MM and a CM that is cleavable by a matriptase and an MMP. In some embodiments, a prodomain comprises a CD3ε MM and a CM that is cleavable by a matriptase and an MMP. Provided herein are activatable antibodies (AAs) comprising a prodomain. Also provided herein are nucleotides encoding a prodomain of the invention.

Accordingly, in some embodiments, provided herein is a CD3 AA comprising: (a) an antibody or antigen binding fragment thereof (AB) that specifically binds to the epsilon chain of CD3 (CD3ε), wherein the antibody comprises a heavy chain domain as set forth in SEQ ID NO: 2 or SEQ ID NO: 3 or comprises a light chain domain as set forth in SEQ ID NO: 1 or SEQ ID NO: 4; and (b) a prodomain, wherein the prodomain comprises a (i) masking moiety (MM) coupled to the AB, wherein the MM reduces or inhibits the binding of the AB to the CD3ε when the AA is in an uncleaved state; and (ii) a cleavable moiety (CM) coupled to the AB, wherein the CM is a polypeptide that functions as a substrate for a protease.

In some embodiments, provided herein is a CD3 AA comprising: (a) an antibody or antigen binding fragment thereof (AB) that specifically binds to CD3ε; and (b) a prodomain, wherein the prodomain comprises: (i) a masking moiety (MM) coupled to the AB, wherein the MM reduces or inhibits the binding of the AB to the CD3ε when the AA is in an uncleaved state, wherein the MM comprises the amino acid sequence SEQ ID NO: 105 or SEQ ID NO: 106 or SEQ ID NO: 107; and (ii) a cleavable moiety (CM) coupled to the AB, wherein the CM is a polypeptide that functions as a substrate for a protease.

In some embodiments, provided herein is a CD3 AA comprising: (a) at least one scFv comprising a light chain variable region (VL) linked to a heavy chain variable region (VH), wherein the VL is linked to the VH by a linker comprising amino acid sequence SEQ ID NO: 108 or SEQ ID NO: 98; and (b) a prodomain comprising: (i) a masking moiety (MM) coupled to the scFv, wherein the MM reduces or inhibits the binding of the scFV to its target when the AA is in an uncleaved state; and (ii) a cleavable moiety (CM) coupled to the scFv, wherein the CM is a polypeptide that functions as a substrate for a protease.

Accordingly, in some embodiments, provided herein is a CD3 AA, which comprises the following structure, when not activated: (a) an antibody or antigen binding fragment thereof (AB) that specifically binds to the epsilon chain of CD3 (CD3ε) when activated, wherein the antibody comprises a heavy chain domain 70%, 80%, 90%, 95%, 99%, or 100% similar to a sequence as set forth in SEQ ID NO: 2 or SEQ ID NO: 3 or comprises a light chain domain 70%, 80%, 90%, 95%, 99%, or 100% similar to a sequence as set forth in as set forth in SEQ ID NO: 1 or SEQ ID NO: 4; and (b) a prodomain, wherein the prodomain comprises a (i) masking moiety (MM) coupled to the AB, wherein the MM reduces or inhibits the binding of the AB to the CD3ε when the AA is in an uncleaved state; and (ii) a cleavable moiety (CM) coupled to the AB, wherein the CM is a polypeptide that functions as a substrate for a protease. The AB may comprise at least one of the CDR sequences provided in Table 2. The AB may comprise the six CDR sequences provided in Table 2.

In some embodiments, provided herein is a CD3ε AA, when activated, specifically binds to a target, and wherein said AA, when not activated, comprises the following structure: (a) an antibody or antigen binding fragment thereof (AB) that specifically binds to CD3ε; and (b) a prodomain comprising: (i) a masking moiety (MM) coupled to the AB, wherein the MM reduces or inhibits the binding of the AB to the CD3ε when the AA is in an uncleaved state, wherein the MM comprises the amino acid sequence SEQ ID NO: 105 or SEQ ID NO: 106; or SEQ ID NO: 107 and (ii) a cleavable moiety (CM) coupled to the AB, wherein the CM is a polypeptide that functions as a substrate for a protease.

In some embodiments, provided herein is a AA, when activated, specifically binds to a target, and wherein said AA, when not activated, comprises the following structure: (a) an antibody or antigen binding fragment thereof (AB) that specifically binds to CD3ε; and (b) a prodomain comprising: (i) a masking moiety (MM) coupled to the AB, wherein the MM reduces or inhibits the binding of the AB to the CD3ε when the AA is in an uncleaved state, wherein the MM comprises the amino acid sequence SEQ ID NO: 105 or SEQ ID NO: 106; or SEQ ID NO: 107 and (ii) a cleavable moiety (CM) coupled to the AB, wherein the CM is a polypeptide that functions as a substrate for a protease.

In some embodiments, the AB of the CD3 AA is any one of the CD3 antibodies described in the preceding section.

In some embodiments, the AB of the CD3 AA comprises a heavy chain variable domain as set forth in SEQ ID NO: 2.

In some embodiments, the AB of the CD3 AA comprises a heavy chain variable domain as set forth in SEQ ID NO: 3.

In some embodiments, the AB of the CD3 AA comprises a light chain variable domain as set forth in SEQ ID NO: 1.

In some embodiments, the AB of the CD3 AA comprises a light chain variable domain as set forth in SEQ ID NO: 4.

In some embodiments, the AB of the CD3 AA comprises a heavy chain variable domain as set forth in SEQ ID NO: 2 and a light chain domain as set forth in SEQ ID NO: 1.

In some embodiments, the AB of the CD3 AA comprises a heavy chain variable domain as set forth in SEQ ID NO: 3 and a light chain domain as set forth in SEQ ID NO: 1.

In some embodiments, the AB of the CD3 AA comprises a heavy chain variable domain as set forth in SEQ ID NO: 3 and a light chain domain as set forth in SEQ ID NO: 4

In some embodiments, the AB is a scFv comprising a light chain variable region (VL) linked to a heavy chain variable region (VH), wherein the VL is linked to the VH by a linker comprising amino acid sequence SEQ ID NO: 108. Exemplary sequences with such a linker are provided in Table 1.

In some embodiments, the AB is a scFv comprising a light chain variable region (VL) linked to a heavy chain variable region (VH), wherein the VL is linked to the VH by a linker comprising amino acid sequence SEQ ID NO: 98. Exemplary sequences with such a linker are provided in Table 1.

In some embodiments, the MM of the CD3 AA comprises any one of the sequences set forth in Table 3.

Exemplary CD3 masking moieties (MMs) of the invention are provided in Table 3.

In some embodiments, the MM of the CD3 AA comprises the sequence set forth in SEQ ID NO: 12. In some embodiments, the MM of the CD3 AA is the sequence set forth in SEQ ID NO: 10. In some embodiments, the MM of the CD3 AA is the sequence set forth in SEQ ID NO: 11. In some embodiments, the MM of the CD3 AA comprises the sequence set forth in SEQ ID NO: 105. In some embodiments, the MM of the CD3 AA comprises the sequence set forth in SEQ ID NO: 106. In some embodiments, the MM of the CD3 AA comprises the sequence set forth in SEQ ID NO: 107.

TABLE 3

| MM | Name | AA sequence | SEQ ID NO: |
|---|---|---|---|
| CD3 MM | JF15865 | MMYCGGNEVLCGPRV | SEQ ID NO: 10 |
| CD3 MM | JP15003 | GYRWGCEWNCGGITT | SEQ ID NO: 11 |
| CD3 MM | h20GG | GYLWGCEWNCGGITT | SEQ ID NO: 12 |
| CD3 MM | hCD1 | MMYCGGNEIFCEPRG | SEQ ID NO: 105 |
| CD3 MM | hCD13 | GYGWGCEWNCGGSSP | SEQ ID NO: 106 |
| CD3 MM | hCD1 | MMYCGGNEIFCGPRG | SEQ ID NO: 107 |
| | variant | | |

In some embodiments, the CM of the CD3 AA comprises any one of the sequences set forth in Table 4. Exemplary cleavable moieties (CMs) of the invention are provided in Table 4. In some embodiments, a cleavable moiety of the CD3 AA comprises any of the amino acid sequences selected from the group consisting of SEQ ID NO:13-17.

In some embodiments, the CM of an AA of the disclosure comprises any one of the sequences set forth in Table 4-1. In some embodiments, the CM of an AA of the disclosure comprises any of the amino acid sequences selected from the group consisting of SEQ ID NO:18-56.

TABLE 4

| CM | | |
|---|---|---|
| Name | AA sequence | SEQ ID NO: |
| 0001 | LSGRSDNH | SEQ ID NO: 13 |
| 0011 | LSGRSDDH | SEQ ID NO: 14 |
| 2001 | ISSGLLSGRSDNH | SEQ ID NO: 15 |
| 2008 | ISSGLLSGRSDQH | SEQ ID NO: 16 |
| 2006 | ISSGLLSGRSDDH | SEQ ID NO: 17 |

TABLE 4-1

| CM Name | AA sequence | SEQ ID NO: |
|---|---|---|
| 0001 | LSGRSDNH | SEQ ID NO: 18 |
| 0002 | LSGRSGNH | SEQ ID NO: 19 |
| 0003 | TSTSGRSANPRG | SEQ ID NO: 20 |
| 1001 | ISSGLLSS | SEQ ID NO: 21 |
| 1002 | QNQALRMA | SEQ ID NO: 22 |
| 1003 | VHMPLGFLGP | SEQ ID NO: 23 |
| 1004 | AVGLLAPP | SEQ ID NO: 24 |
| 0021 | LSGRSDIH | SEQ ID NO: 26 |
| 0031 | LSGRSDQH | SEQ ID NO: 27 |
| 0041 | LSGRSDTH | SEQ ID NO: 28 |
| 0051 | LSGRSDYH | SEQ ID NO: 29 |
| 0061 | LSGRSDNP | SEQ ID NO: 30 |
| 0071 | LSGRSANP | SEQ ID NO: 31 |
| 0081 | LSGRSANI | SEQ ID NO: 32 |
| 0091 | LSGRSDNI | SEQ ID NO: 33 |
| 2001 | ISSGLLSGRSDNH | SEQ ID NO: 34 |
| 2002 | ISSGLLSGRSGNH | SEQ ID NO: 35 |
| 2003 | ISSGLLSGRSANPRG | SEQ ID NO: 36 |
| 2005 | AVGLLAPPSGRSANPRG | SEQ ID NO: 37 |
| 2006 | ISSGLLSGRSDDH | SEQ ID NO: 38 |
| 2007 | ISSGLLSGRSDIH | SEQ ID NO: 39 |
| 2009 | ISSGLLSGRSDTH | SEQ ID NO: 41 |
| 2010 | ISSGLLSGRSDYH | SEQ ID NO: 42 |
| 2011 | ISSGLLSGRSDNP | SEQ ID NO: 43 |
| 2012 | ISSGLLSGRSANP | SEQ ID NO: 44 |
| 2013 | ISSGLLSGRSANI | SEQ ID NO: 45 |
| 2014 | ISSGLLSGRSDNI | SEQ ID NO: 46 |
| 3001 | AVGLLAPPGGLSGRSDNH | SEQ ID NO: 47 |
| 3006 | AVGLLAPPGGLSGRSDDH | SEQ ID NO: 48 |
| 3007 | AVGLLAPPGGLSGRSDIH | SEQ ID NO: 49 |
| 3008 | AVGLLAPPGGLSGRSDQH | SEQ ID NO: 50 |
| 3009 | AVGLLAPPGGLSGRSDTH | SEQ ID NO: 51 |
| 3010 | AVGLLAPPGGLSGRSDYH | SEQ ID NO: 52 |
| 3011 | AVGLLAPPGGLSGRSDNP | SEQ ID NO: 53 |
| 3012 | AVGLLAPPGGLSGRSANP | SEQ ID NO: 54 |
| 3013 | AVGLLAPPGGLSGRSANI | SEQ ID NO: 55 |
| 3014 | AVGLLAPPGGLSGRSDNI | SEQ ID NO: 56 |

3. Antibodies with Fc Mutations

Provided herein are IgG1 antibodies that that have Fc mutations or antibody fragments containing antigen-binding domains (e.g. scFv, Fab, F(ab′)2) linked to a Fc domain, wherein the Fc exhibits reduced effector function (referred to herein as Fc variants).

The antibodies that comprise these Fc mutations result in reduced effector function, while maintaining target binding affinity. Accordingly, provided herein are antibodies that bind to a target of interest, wherein the antibody is an IgG1 antibody or an antibody fragment linked to an Fc, wherein the Fc region comprises an amino acid substitution in at least one of amino acid positions L234, L235, and P331, as numbered by the EU index as set forth in Kabat, such that the antibody has reduced effector function. In some embodiments, the amino acid substitution is any one or more of L234F, L235E, and P331S. In some embodiments, there is an additional mutation in N297. In some embodiments, the amino acid substitution is N297Q or N297A.

In some embodiments, the Fc is selected from the Fc sequences presented in Table 4-2.

TABLE 4-2

| Name | |
|---|---|
| SEQ ID | |
| NO: | AA Sequence |
| Fc-N297X | ASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 113) | DKKVEPKSCDKTHTCPPCPAPELLGGPSVFLFPPKPKDTLMISRTPEV |
| | TCVVVDVSHEDPEVKFNWYVDGVEVHNAKTKPREEQYXSTYRVV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |
| Fc-N297 | QASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 114) | DKKVEPKSCDKTHTCPPCPAPELLGGPSVFLFPPKPKDTLMISRTPEV |
| | TCVVVDVSHEDPEVKFNWYVDGVEVHNAKTKPREEQYQSTYRVV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |
| Fc-L234X | ASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 115) | DKKVEPKSCDKTHTCPPCPAPEXLGGPSVFLFPPKPKDTLMISRTPE |
| | VTCVVVDVSHEDPEVKFNWYVDGVEVHNAKTKPREEQYNSTYRV |
| | VSVLTVLHQDWLNGKEYKCKVSNKALPAPIEKTISKAKGQPREPQV |
| | YTLPPSREEMTKNQVSLTCLVKGFYPSDIAVEWESNGQPENNYKTT |
| | PPVLDSDGSFFLYSKLTVDKSRWQQGNVFSCSVMHEALHNHYTQK |
| | SLSLSPGK |
| Fc-L234F | QVQLKQSGPGLVQPSQSLSITCTVSGFSLTNYGVHWVRQSPGKGLE |
| (SEQ ID | WLGVIWSGGNTDYNTPFTSRLSINKDNSKSQVFFKMNSLQSQDTAI |
| NO: 116) | YYCARALTYYDYEFAYWGQGTLVTVS(S/A)ASTKGPSVFPLAPSSK |
| | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPEFLGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDK |
| | SRWQQGNVFSCSVMHEALHNHYTQKSLSLSPGK |
| Fc-L235X | QVQLKQSGPGLVQPSQSLSITCTVSGFSLTNYGVHWVRQSPGKGLE |
| (SEQ ID | WLGVIWSGGNTDYNTPFTSRLSINKDNSKSQVFFKMNSLQSQDTAI |
| NO: 117) | YYCARALTYYDYEFAYWGQGTLVTVS(S/A)ASTKGPSVFPLAPSSK |
| | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELXGGPSVFLFPPKPKDTLMISRTPEVTCVVVDVSHEDPE |
| | VKFNWYVDGVEVHNAKTKPREEQYNSTYRVVSVLTVLHQDWLNG |
| | KEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLT |
| | VDKSRWQQGNVFSCSVMHEALHNHYTQKSLSLSPGK |
| Fc-L235E | ASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 118) | DKKVEPKSCDKTHTCPPCPAPELEGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAPIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |
| Fc-P331X | ASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 119) | DKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPAXIEKTISKAKGQPREPQV |
| | YTLPPSREEMTKNQVSLTCLVKGFYPSDIAVEWESNGQPENNYKTT |
| | PPVLDSDGSFFLYSKLTVDKSRWQQGNVFSCSVMHEALHNHYTQK |
| | SLSLSPGK |
| Fc-P331S | ASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 120) | DKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPASIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |
| Fc-Fcmt3 | ASTKGPSVFPLAPSSKSTSGGTAALGCLVKDYFPEPVTVSWNSGALT |
| (SEQ ID | SGVHTFPAVLQSSGLYSLSSVVTVPSSSLGTQTYICNVNHKPSNTKV |
| NO: 121) | DKKVEPKSCDKTHTCPPCPAPEFEGGPSVFLFPPKPKDTLMISRTPEV |
| | TCVVVDVSHEDPEVKFNWYVDGVEVHNAKTKPREEQYNSTYRVV |
| | SVLTVLHQDWLNGKEYKCKVSNKALPASIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |
| C225v5Fcmt4 | ASTKGPSVFPLAPSSKSTSGGTAALGCLVKDYFPEPVTVSWNSGALT |
| HC | SGVHTFPAVLQSSGLYSLSSVVTVPSSSLGTQTYICNVNHKPSNTKV |
| (SEQ ID | DKKVEPKSCDKTHTCPPCPAPEFEGGPSVFLFPPKPKDTLMISRTPEV |
| NO: 122) | TCVVVDVSHEDPEVKFNWYVDGVEVHNAKTKPREEQYQSTYRVV |
| | SVLTVLHQDWLNGKEYKCKVSNKALPASIEKTISKAKGQPREPQVY |
| | TLPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |

Antibodies, AAs, bispecific antibodies, and BAAs comprising these Fc mutations are provided herein.

In some embodiments, the Fc domain of such antibodies, AAs, bispecific antibodies, and BAAs comprise any one of the sequences set forth in SEQ ID NO: 115, SEQ ID NO: 116, SEQ ID NO: 117, SEQ ID NO: 118, SEQ ID NO: 119, SEQ ID NO: 120, SEQ ID NO: 121 and SEQ ID NO: 122, as set forth in Table 4-2.

In some embodiments, such Fc variant-containing AAs and BAAs can bind an immune effector cell. In some embodiments, they can bind a target selectively located on an immune effector cell. In some embodiments, they can bind CD3. In some embodiments, they can bind any target listed in Table 9. In some embodiments, they can bind EGFR.

Accordingly in some embodiments, provided herein is an activatable antibody (AA) comprising: an antibody (AB) that specifically binds a target, wherein the antibody is an IgG1 antibody, and wherein the Fc region of the antibody comprises an amino acid substitution in at least one of amino acid positions L234, L235, and P331, as numbered by the EU index as set forth in Kabat, such that the AA has reduced effector function; and a prodomain, wherein the prodomain comprises:

(i) a masking moiety (MM) coupled to the AB, wherein the MM reduces or inhibits the binding of the AB to the target when the AA is in an uncleaved state; and

(ii) a cleavable moiety (CM) coupled to the AB, wherein the CM is a polypeptide that functions as a substrate for a protease.

Accordingly in some embodiments, provided herein is an activatable antibody (AA) comprising: an antibody (AB) that specifically binds a target, wherein the antibody is an IgG1 antibody, and wherein the Fc region of the antibody comprises an amino acid substitution in at least one of amino acid positions L234, L235, and P331, as numbered by the EU index as set forth in Kabat, such that the AA has reduced effector function; and a prodomain, wherein the prodomain comprises:

(i) a masking moiety (MM) coupled to the AB, wherein the MM reduces or inhibits the binding of the AB to the target when the AA is in an uncleaved state, wherein the MM comprises the amino acid sequence SEQ ID NO: 105 or SEQ ID NO: 106 or SEQ ID NO: 107; and

(ii) a cleavable moiety (CM) coupled to the AB, wherein the CM is a polypeptide that functions as a substrate for a protease.

In some embodiments, the Fc region comprises amino acid substitutions in at least amino acid positions L234, L235, N297 and P331 (e.g. L234F, L235E, P331S, and/or N297A or N297Q), as numbered by the EU index as set forth in Kabat, such that the AA has reduced effector function. In some embodiments, the target is selected from the group consisting of the targets presented in Table 9.

In some embodiments, provided herein is a bispecific activatable antibody (BAA), wherein said BAA, when activated, specifically binds to two targets (or binds two different epitopes on the same target), and wherein said BAA, when not activated, comprises the following structure:

a) an IgG antibody (AB1) that specifically binds to a first target wherein the AB1 comprises:

(i) two heavy chains (AB1 HCs) and two light chains (AB1 LCs); and

(ii) two first prodomains, each comprising a first masking moiety (MM1) linked to a first cleavable moiety (CM1) in the N-terminal to C-terminal direction, wherein the carboxyl terminus of each first prodomain is linked to the amino terminus of each light chain of the AB1, wherein the MM1 reduces or inhibits the binding of the AB1 to its target; and the CM1 is a polypeptide that functions as a substrate for a first protease,

b) two scFvs (AB2) that each specifically binds CD3ε, wherein each AB2 comprises:

(i) a light chain variable region (VL) linked to a heavy chain variable region (VH), wherein the carboxyl terminus of each AB2 is linked to the amino terminus of each of the AB1 heavy chains, wherein the VL comprises an amino acid sequence as set forth in SEQ ID NO: 1 or SEQ ID NO: 4 and the VH comprises an amino acid sequence as set forth in SEQ ID NO: 2 or SEQ ID NO: 3; and the VL is linked to the VH by a linker (L3) comprising an amino acid sequence selected from the group consisting of SEQ ID NO: 98 and SEQ ID NO:108; and

(ii) two second prodomains, each comprising a second masking moiety (MM2) linked to a second cleavable moiety (CM2), in the N-terminal to C-terminal direction, wherein the carboxyl terminus of each second prodomain is linked to the amino terminus of each AB2 wherein the MM2 reduces the binding of the AB2 to CD3ε; the MM2 comprises an amino acid sequence selected from the group consisting of SEQ ID NO: 12, SEQ ID NO: 105, SEQ ID NO: 106 and SEQ ID NO: 107; and the CM2 is a polypeptide that functions as a substrate for a second protease.

### 4. EGFR Antibodies

Provided herein are antibodies or antigen binding fragments thereof (AB) that specifically bind to EGFR. Exemplary CDR sequences of EGFR-binding antibodies are provided in Table 5.

Provided herein are EGFR antibodies, bispecific antibodies targeting EGFR, AAs capable of binding EGFR upon activation, and BAAs capable of binding EGFR upon activation. In some embodiments, the EGFR antibody comprises the CDRs of Table 5.

In some embodiments, e.g. in a BAA format, provided herein are IgG1 antibodies that specifically bind to the Epidermal Growth Factor Receptor (EGFR) and impart reduced effector function. The antibodies comprise Fc mutations that result in reduced effector function, while maintaining EGFR binding affinity. Accordingly, provided herein are antibodies that bind to EGFR, wherein the antibody is an IgG1 antibody, wherein the antibody comprises an Fc region comprising an amino acid substitution in at least one of amino acid positions L234, L235, and P331, as numbered by the EU index as set forth in Kabat, such that the antibody has reduced effector function. In some embodiments, the amino acid substitution is any one or more of L234F, L235E, and P331S.

In some embodiments, the antibody comprises amino acid substitutions in at least two of amino acid positions L234, L235, and P331.

In some embodiments, the antibody comprises amino acid substitutions at amino acid positions L234, L235, and P331.

In some embodiments, the antibody comprises L234F, L235E, and P331S amino acid substitutions.

In some embodiments, the antibody comprises an Fc region comprising an amino acid substitution at N297 along with an amino acid substitution in at least one of amino acid positions L234, L235, and/or P331. In some embodiments, the Fc region comprises an N297Q mutation. In some embodiments, the Fc region comprises an N297A mutation.

In some embodiments, the antibody comprises L234F, L235E, P331S and N297Q substitutions. In some embodiments, the antibody comprises L234F, L235E, P331S and N297A substitutions.

Exemplary CDR sequences of EGFR-binding antibodies are provided in Table 5, set forth in Kabat.

TABLE 5

| Name | Sequence | SEQ ID NO: |
|---|---|---|
| C225L1 | RASQSIGTNIH | SEQ ID NO: 57 |
| C225L2 | YASESIS | SEQ ID NO: 58 |
| C225L3 | QQNNNWPTT | SEQ ID NO: 59 |
| C225H1 | NYGVH | SEQ ID NO: 60 |
| C225H2 | VIWSGGNTDYNTPFTS | SEQ ID NO: 61 |
| C225H3 | ALTYYDYEFAY | SEQ ID NO: 62 |

Exemplary amino acid sequences of EGFR-binding antibodies are provided in Table 6. (VL and VH denote the variable light and variable heavy chains, respectively; LC and HC denote the light and heavy chains, respectively).

In some embodiments, the EGFR antibodies comprise any one of the sequences provided in Table 6.

In some embodiments, the heavy chain of the EGFR antibody comprises any one of the sequences set forth in SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76, as set forth in Table 6.

The notation Fcmt3 comprises a triple point mutation, wherein the Fc region of the heavy chain of the EGFR antibody comprises the following three point mutations: L234F, L235E, and P331S. Accordingly, in some embodiments, the EGFR antibody comprises a heavy chain with an amino acid sequence set forth in SEQ ID NO: C225v5Fcmt3 HC. In some embodiments, the Fc region of the heavy chain of the EGFR antibody comprises a fourth point mutation, N297Q. The notation Fcmt4 comprises the Fcmt3 triple point mutation and the fourth point mutation, N297Q. Accordingly, in such embodiments, the EGFR antibody comprises a heavy chain with an amino acid sequence set forth in SEQ ID NO: 76.

In some embodiments, the AB comprises a sequence 70%, 80%, 90%, 95%, 99%, or 100% similar to a heavy chain variable domain set out in SEQ ID NO: 64, and/or comprises a sequence 70%, 80%, 90%, 95%, 99%, or 100% similar to a light chain variable domain as set out in SEQ ID NO: 63, and specifically binds to EGFR. In some embodiments, the AB comprises a sequence 70%, 80%, 90%, 95%, 99%, or 100% similar to a heavy chain variable domain as set out in SEQ ID NO: 64, and/or comprises a sequence 70%, 80%, 90%, 95%, 99%, or 100% similar to a light chain variable domain as set out in SEQ ID NO: 63, comprises the six CDR sequences provided in Table 5, and specifically binds to EGFR. In some embodiments, the AB comprises an Fc region as set out above or in Table 6.

TABLE 6

| Name | |
|---|---|
| SEQ ID | |
| NO: | AA Sequence |
| C225V5-VL | QILLTQSPVILSVSPGERVSFSCRASQSIGTNIHWYQQRTNGSPRLLIK |
| (SEQ ID | YASESISGIPSRFSGSGSGTDFTLSINSVESEDIADYYCQQNNNWPTT |
| NO: 63) | FGAGTKLELK |
| C225V5-VH | QVQLKQSGPGLVQPSQSLSITCTVSGFSLTNYGVHWVRQSPGKGLE |
| (SEQ ID | WLGVIWSGGNTDYNTPFTSRLSINKDNSKSQVFFKMNSLQSQDTAI |
| NO: 64) | YYCARALTYYDYEFAYWGQGTLVTVS(S/A) |
| C225v5 LC | QILLTQSPVILSVSPGERVSFSCRASQSIGTNIHWYQQRTNGSPRLLIK |
| (SEQ ID | YASESISGIPSRFSGSGSGTDFTLSINSVESEDIADYYCQQNNNWPTT |
| NO: 65) | FGAGTKLELKRTVAAPSVFIFPPSDEQLKSGTASVVCLLNNFYPREA |
| | KVQWKVDNALQSGNSQESVTEQDSKDSTYSLSSTLTLSKADYEKH |
| | KVYACEVTHQGLSSPVTKSFNRGEC |
| C225v5 HC | QVQLKQSGPGLVQPSQSLSITCTVSGFSLTNYGVHWVRQSPGKGLE |
| (SEQ ID | WLGVIWSGGNTDYNTPFTSRLSINKDNSKSQVFFKMNSLQSQDTAI |
| NO: 66) | YYCARALTYYDYEFAYWGQGTLVTVS(S/A)ASTKGPSVFPLAPSSK |
| | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| C225v5N29 | QVQLKQSGPGLVQPSQSLSITCTVSGFSLTNYGVHWVRQSPGKGLE |
| 7X HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 67) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEV |
| | KFNWYVDGVEVHNAKTKPREEQYXSTYRVVSVLTVLHQDWLNGK |
| | EYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| C225v5N29 | QVQLKQSGPGLVQPSQSLSITCTVSGFSLTNYGVHWVRQSPGKGLE |
| 7Q HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 68) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEV |
| | KFNWYVDGVEVHNAKTKPREEQYQSTYRVVSVLTVLHQDWLNGK |
| | EYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| C225v5 | QVQLKQSGPGLVQPSQSLSITCTVSGFSLTNYGVHWVRQSPGKGLE |
| L234X HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 69) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPEXLGGPSVFLFPPKPKDTLMISRTPEVTCVVVDVSHEDPE |
| | VKFNWYVDGVEVHNAKTKPREEQYNSTYRVVSVLTVLHQDWLNG |
| | KEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSK |
| | LTVDKSRWQQGNVFSCSVMHEALHNHYTQKSLSLSPGK |
| C225v5 | QVQLKQSGPGLVQPSQSLSITCTVSGFSLTNYGVHWVRQSPGKGLE |
| L234F HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 70) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPEFLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFY |
| | PSDIAVEWESNGQPENNYKTTPPVLDSDGSFFLYSKLTVDKSRWQQGNVF |
| | SCSVMHEALHNHYTQKSLSLSPGK |
| C225v5 | QVQLKQSGPGLVQPSQSLSITCTVSGFSLTNYGVHWVRQSPGKGLE |
| L235X HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 71) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELXGGPSVFLFPPKPKDTLMISRTPEVTCVVVDVSHEDPE |
| | VKFNWYVDGVEVHNAKTKPREEQYNSTYRVVSVLTVLHQDWLNG |
| | KEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSK |
| | LTVDKSRWQQGNVFSCSVMHEALHNHYTQKSLSLSPGK |
| C225v5 | QVQLKQSGPGLVQPSQSLSITCTVSGFSLTNYGVHWVRQSPGKGLE |
| L235E HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 72) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELEGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| C225v5 | QVQLKQSGPGLVQPSQSLSITCTVSGFSLTNYGVHWVRQSPGKGLE |
| P331X HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 73) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPAXIEKTISKAKGQPREPQVYTLPPSREEMTKNQV |
| | SLTCLVKGFYPSDIAVEWESNGQPENNYKTTPPVLDSDGSFFLYSKL |
| | TVDKSRWQQGNVFSCSVMHEALHNHYTQKSLSLSPGK |
| C225v5 | QVQLKQSGPGLVQPSQSLSITCTVSGFSLTNYGVHWVRQSPGKGLE |
| P331S HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 74) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPASIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| C225v5Fcmt3 | QVQLKQSGPGLVQPSQSLSITCTVSGFSLTNYGVHWVRQSPGKGLE |
| HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 75) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPEFEGGPSVFLFPPKPKDTLMISRTPEVTCVVVDVSHEDPEV |
| | KFNWYVDGVEVHNAKTKPREEQYNSTYRVVSVLTVLHQDWLNGK |
| | EYKCKVSNKALPASIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| C225v5Fcmt4 | QVQLKQSGPGLVQPSQSLSITCTVSGFSLTNYGVHWVRQSPGKGLE |
| HC | WLGVIWSGGNTDYNTPFTSRLSINKDNSKSQVFFKMNSLQSQDTAI |
| (SEQ ID | YYCARALTYYDYEFAYWGQGTLVTVS(S/A)ASTKGPSVFPLAPSSK |
| NO: 76) | STSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHT |
| | CPPCPAPEFEGGPSVFLFPPKPKDTLMISRTPEVTCVVVDVSHEDPEV |
| | KFNWYVDGVEVHNAKTKPREEQYQSTYRVVSVLTVLHQDWLNGK |
| | EYKCKVSNKALPASIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |
| SynFcmt4 | QVTLRESGPALVKPTQTLTLTCTFSGFSLSTSGMSVGWIRQPPGKAL |
| HC | EWLADIWWDDKKDYNPSLKSRLTISKDTSKNQVVLKVTNMDPADT |
| (SEQ ID | ATYYCARSMITNWYFDVWGAGTTVTVS(S/A)ASTKGPSVFPLAPSS |
| NO: 77) | KSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHT |
| | CPPCPAPEFEGGPSVFLFPPKPKDTLMISRTPEVTCVVVDVSHEDPEV |
| | KFNWYVDGVEVHNAKTKPREEQYQSTYRVVSVLTVLHQDWLNGK |
| | EYKCKVSNKALPASIEKTISKAKGQPREPQVYTLPPSREEMTKNQVS |
| | LTCLVKGFYPSDIAVEWESNGQPENNYKTTPPVLDSDGSFFLYSKLT |
| | VDKSRWQQGNVFSCSVMHEALHNHYTQKSLSLSPGK |

### 5. Activatable EGFR Antibodies

In some embodiments, any one of the EGFR antibodies provided herein are in an AA format (EGFR AAs). Accordingly provided herein are AAs comprising antibodies or antigen binding fragments thereof (AB) that specifically bind to EGFR. Exemplary CDR sequences of EGFR-binding antibodies are provided in Table 5.

In some embodiments, the AA comprises: (a) any antibody or an antigen binding fragment thereof (AB) that specifically binds to Epidermal Growth Factor Receptor (EGFR); and (b) a prodomain, wherein the prodomain comprises (i) a masking moiety (MM) coupled to the AB, wherein the MM reduces or inhibits the binding of the AB to the EGFR when the AA is in an uncleaved state, and wherein the MM comprises an amino acid sequence selected from the group consisting of sequences presented in Table 7; and (ii) a cleavable moiety (CM) coupled to the AB, wherein the CM is a polypeptide that functions as a substrate for a protease.

Exemplary EGFR masking moieties (MMs) of the invention are provided in Table 7 and Table 8.

TABLE 7

| MM | Name | AA sequence | SEQ ID NO: |
|---|---|---|---|
| EGFR MM | CF41 | LSCEGWAMNREQCRA | SEQ ID NO: 78 |
| EGFR MM | CF08 | PPLECNTKSMCSKHD | SEQ ID NO: 79 |
| EGFR MM | CF13 | DRDCRGRRARCQQEG | SEQ ID NO: 80 |
| EGFR MM | CF19 | FTCEGWAMNREQCRT | SEQ ID NO: 81 |
| EGFR MM | CF22 | GRCPPSRDIRFCTYM | SEQ ID NO: 82 |
| EGFR MM | CF46 | FSCEGWAMNRSQCRT | SEQ ID NO: 83 |
| EGFR MM | CF48 | FTCEGWAMNRDQCRT | SEQ ID NO: 84 |

TABLE 8

| MM | Name | AA sequence | SEQ ID NO: |
|---|---|---|---|
| EGFR MM | 3954 | CISPRGCPDGPYVMY | SEQ ID NO: 85 |
| EGFR MM | 3954a | CISPRGCPDGPYVM | SEQ ID NO: 86 |
| EGFR MM | 3960 | CISPRGC | SEQ ID NO: 87 |

In some embodiments, the MM of the EGFR AA comprises the amino acid sequence of SEQ ID NO: 78. In some embodiments, the MM of the EGFR AA comprises the amino acid sequence of SEQ ID NO: 85.

In some embodiments, the CM of the EGFR AA comprises an amino acid sequence selected from the group consisting of sequences presented in Table 4. In some embodiments, the CM comprises the amino acid sequence of SEQ ID NO: 14. In some embodiments, the CM comprises the amino acid sequence of SEQ ID NO: 16. In some embodiments, provided herein is an activatable antibody (AA) comprising: (a) an antibody that specifically binds to Epidermal Growth Factor Receptor (EGFR), wherein the antibody is an IgG1 antibody, and wherein the Fc region of the antibody comprises an amino acid substitution in at least one of amino acid positions L234, L235, and P331, as numbered by the EU index as set forth in Kabat, such that the AA has reduced effector function; (b) a masking moiety (MM) coupled to the AB, wherein the MM reduces or inhibits the binding of the AB to the EGFR when the AA is in an uncleaved state; and (c) a cleavable moiety (CM) coupled to the AB, wherein the CM is a polypeptide that functions as a substrate for a protease. The EGFR IgG1 antibodies can be any of the IgG1 antibodies described in the immediately preceding section. In some embodiments, the MM comprises an amino acid sequence selected from the group consisting of sequences presented in Table 7.

In an exemplary embodiment, provided herein is an activatable antibody (AA) comprising: (a) an antibody (AB) that specifically binds to Epidermal Growth Factor Receptor (EGFR), wherein the AB is an IgG1 antibody, and wherein the Fc region of the AB comprises an amino acid substitution in at least one of amino acid positions L234, L235, and P331, as numbered by the EU index as set forth in Kabat, such that the AA has reduced effector function; (b) a masking moiety (MM) coupled to the AB, wherein the MM reduces or inhibits the binding of the AB to the EGFR when the AA is in an uncleaved state; and (c) a cleavable moiety (CM) coupled to the AB, wherein the CM is a polypeptide that functions as a substrate for a protease. In some embodiments, the amino acid substitution is any one or more of L234F, L235E, and P331S. In some embodiments, the AB comprises amino acid substitutions in at least two of amino acid positions L234, L235, and P331. In some embodiments, the AB comprises amino acid substitutions at amino acid positions L234, L235, and P331. In some embodiments, the AB comprises L234F, L235E, and P331S amino acid substitutions. In some embodiments, the AB comprises an Fc region comprising an amino acid substitution at N297. In some embodiments, the Fc region comprises an N297Q mutation. In some embodiments, the AB comprises L234F, L235E, P331S, and N297Q amino acid substitutions. In some embodiments, the MM comprises an amino acid sequence selected from the group consisting of sequences presented in Table 7 or Table 8. In some embodiments, the MM comprises the amino acid sequence of SEQ ID NO: 78. In some embodiments, the MM comprises the amino acid sequence of SEQ ID NO: 85. In some embodiments, the CM comprises an amino acid sequence selected from the group consisting of sequences presented in Table 4. In some embodiments, the CM comprises the amino acid sequence of SEQ ID NO: 14. In some embodiments, the CM comprises the amino acid sequence of SEQ ID NO: 16. In some embodiments, the AA is part of a BAA.

### 6. Bispecific Activatable Antibodies (BAAs)

Provided herein are BAAs (bispecific AAs, BAAs), wherein said bispecific AA, when activated, specifically binds to two targets (e.g. binds two different targets, or binds two different epitopes on the same target) and can comprise the exemplary structure provided in FIG. 17.

In some embodiments, the first target is selected from the group consisting of the targets presented in Table 9 and the second target is selected from the group consisting of the targets presented in Table 9.

As generally provided herein, and as described above in the section describing AAs, the BAAs of the invention comprise MM-CM constructs, also referred to herein as a prodomain. Accordingly, as used herein, the term “prodomain” refers to a polypeptide comprising a masking moiety (MM) and a cleavable moiety (CM). In some embodiments, the MM and the CM are separated by a linker, referred to herein as L1. In some embodiments, the prodomain comprises a linker at the carboxyl terminus of the CM; this linker, referred to herein as L2, links the CM of the prodomain to the AB. In some embodiments, the prodomain comprises a linker between MM and CM and a linker after CM. In some embodiments, the MM and the CM are not separated by a linker. In certain embodiments a prodomain comprises one of the following formulae (where the formula below represents an amino acid sequence in either N- to C-terminal direction or C- to N-terminal direction): (MM)-L1-(CM), (MM)-(CM)-L2, (MM)-L1-(CM)-L2, or (MM)-(CM). In exemplary embodiments, a prodomain comprises an EGFR MM and a CM cleavable by a matriptase or MMP; or a CD3ε MM and a CM cleavable by a matriptase or MMP. In some embodiments, a prodomain comprises an EGFR MM and a CM that is cleavable by a matriptase and an MMP. In some embodiments, a prodomain comprises a CD3ε MM and a CM that is cleavable by a matriptase and an MMP. Provided herein are bispecific activatable antibodies (BAAs) comprising a prodomain. Also provided herein are nucleotides encoding a prodomain of the invention.

In some embodiments, provided herein is a BAA, wherein said BAA, when activated, specifically binds to two targets (e.g. two different targets; or two different epitopes on the same target), and wherein said BAA, when not activated, comprises the following structure:
a) an IgG antibody (AB1) that specifically binds to a first target wherein the AB1 comprises:
   i. two heavy chains (AB1 HCs) and two light chains (AB1 LCs); and
      ii. two first prodomains, each comprising a first masking moiety (MM1) linked to a first cleavable moiety (CM1) in the N-terminal to C-terminal direction, wherein the carboxyl terminus of each first prodomain is linked to the amino terminus of each light chain of the AB1, wherein
         the MM1 reduces or inhibits the binding of the AB1 to its target; and
         the CM1 is a polypeptide that functions as a substrate for a first protease,
b) two scFvs (AB2) that each specifically binds CD3ε, wherein each AB2 comprises:
   i. a light chain variable region (VL) linked to a heavy chain variable region (VH), wherein the carboxyl terminus of each AB2 is linked to the amino terminus of each of the AB1 heavy chains, wherein the VL comprises an amino acid sequence as set forth in SEQ ID NO: 1 or SEQ ID NO: 4 and the VH comprises an amino acid sequence as set forth in SEQ ID NO: 2 or SEQ ID NO: 3; and the VL is linked to the VH by a linker (L3) comprising an amino acid sequence selected from the group consisting of SEQ ID NO: 98 and SEQ ID NO: 108; and
      ii. two second prodomains, each comprising a second masking moiety (MM2) linked to a second cleavable moiety (CM2), in the N-terminal to C-terminal direction, wherein the carboxyl terminus of each second prodomain is linked to the amino terminus of each AB2 wherein
         the MM2 reduces the binding of the AB2 to CD3ε;
         the MM2 comprises an amino acid sequence selected from the group consisting of SEQ ID NO: 12, SEQ ID NO: 105, SEQ ID NO: 106 and SEQ ID NO: 107, and
         the CM2 is a polypeptide that functions as a substrate for a second protease.

In some embodiments, the VL is linked to the VH by a linker (L3) comprising amino acid sequence SEQ ID NO: 98.

In some embodiments, the VL is linked to the VH by a linker (L3) comprising amino acid sequence SEQ ID NO: 108.

In some embodiments, the MM2 comprises amino acid sequence SEQ ID NO: 12.

In some embodiments, the MM2 comprises amino acid sequence SEQ ID NO: 105.

In some embodiments, the MM2 comprises amino acid sequence SEQ ID NO: 106.

In some embodiments, the MM2 comprises amino acid sequence SEQ ID NO: 107.

In some embodiments, the AB1 binds a tumor target.

In some embodiments, the AB1 binds EGFR. The EGFR-binding AB1 may be any EGFR-binding antibody, or fragment thereof, as disclosed herein.

In some embodiments, the AB2 may be any CD3-binding antibody, or fragment thereof, as disclosed herein.

In some embodiments, the MM1 comprises an amino acid sequence selected from the group consisting of sequences presented in Table 7 or Table 8.

In some embodiments, the MM1 comprises an amino acid sequence selected from the group consisting of SEQ ID NO: 78 and SEQ ID NO: 85.

In some embodiments, the MM1 comprises amino acid sequence SEQ ID NO: 78.

In some embodiments, the MM1 comprises amino acid sequence SEQ ID NO: 85.

In some embodiments, the CM1 comprises the amino acid sequence of any one of the CMs listed in Table 4 or Table 4-1.

In some embodiments, the CM2 comprises the amino acid sequence of any one of the CMs listed in Table 4 or Table 4-1.

In some embodiments, the CM1 or CM2 comprises the amino acid sequence of any one of the CMs listed in Table 4 or Table 4-1

In some embodiments, the CM1 comprises the amino acid sequence of SEQ ID NO: 14, SEQ ID NO: 16, or SEQ ID NO: 17.

In some embodiments, the CM2 comprises the amino acid sequence of SEQ ID NO: 14, SEQ ID NO: 16, or SEQ ID NO: 17.

In some embodiments, the CM1 or CM2 comprises the amino acid sequence of SEQ ID NO: 14, SEQ ID NO: 16, or SEQ ID NO: 17.

In some embodiments, the CM1 comprises an amino acid sequence selected from the group consisting of SEQ ID NO: 14 and SEQ ID NO: 16.

In some embodiments, the CM2 comprises an amino acid sequence selected from the group consisting of SEQ ID NO: 14 and SEQ ID NO: 17.

In some embodiments, the AB1 comprises an Fc region comprising an amino acid substitution in at least one of amino acid positions L234, L235, N297, and P331, as numbered by the EU index as set forth in Kabat, such that the BAA has reduced effector function.

In some embodiments, the AB1 comprises amino acid substitutions in at least two of amino acid positions L234, L235, and P331.

In some embodiments, the AB1 comprises amino acid substitutions at amino acid positions L234, L235, and P331.

In some embodiments, the AB1 comprises L234F, L235E, and P331S amino acid substitutions.

In some embodiments, the AB1 comprises an Fc region comprising an amino acid substitution at N297.

In some embodiments, the AB1 comprises L234F, L235E, P331S, and N297Q amino acid substitutions.

In some embodiments, the heavy chain of AB1 comprises an amino acid sequence selected from the group consisting SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76, as set forth in Table 6.

In some embodiments, the BAA is CI138, comprising the layout and sequence as provided in Tables 11 and 1. In some embodiments, the BAA is CI138, wherein the heavy chain sequence comprises an amino acid sequence selected from the group consisting SEQ ID NO: 155 or SEQ ID NO:124 and the light chain sequence comprises an amino acid sequence selected from the group consisting SEQ ID NO: 132 or SEQ ID NO:140.

In some embodiments, the BAA is CI139, comprising the layout and sequence as provided in Tables 11 and 1. In some embodiments, the BAA is CI139, wherein the heavy chain sequence comprises an amino acid sequence selected from the group consisting SEQ ID NO: 157 or SEQ ID NO:126 and the light chain sequence comprises an amino acid sequence selected from the group consisting SEQ ID NO: 132 or SEQ ID NO:140.

In some embodiments, the BAA is CI158, comprising the layout and sequence as provided in Tables 11 and 1. In some embodiments, the BAA is CI158, wherein the heavy chain sequence comprises an amino acid sequence selected from the group consisting SEQ ID NO: 161 or SEQ ID NO:128 and the light chain sequence comprises an amino acid sequence selected from the group consisting SEQ ID NO: 132 or SEQ ID NO:140.

In some embodiments, the BAA is CI140, comprising the layout and sequence as provided in Tables 11 and 1. In some embodiments, the BAA is CI140, wherein the heavy chain sequence comprises an amino acid sequence selected from the group consisting SEQ ID NO: 159 or SEQ ID NO:25 and the light chain sequence comprises an amino acid sequence selected from the group consisting SEQ ID NO: 132 or SEQ ID NO:140.

In some embodiments, the BAAs provided herein comprise:
c) an IgG antibody (AB1) that specifically binds to a first target wherein the AB1 comprises:
   i. two heavy chains (AB1 HCs) and two light chains (AB1 LCs); and
   ii. a first masking moiety (MM1) linked to a first cleavable moiety (CM1) to form a prodomain (referred to herein interchangeably as a MM1-CM1 construct), wherein the carboxyl terminus of the prodomain is linked to each amino terminus of each light chain of the AB1, wherein
      1. the MM1 inhibits the binding of the AB1 to its target; and
      2. the CM1 is a polypeptide that functions as a substrate for a first protease,
d) two scFvs (AB2) that each specifically bind to a second target wherein each AB2 comprises:
   i. a heavy chain variable region linked to a light chain variable region, wherein the carboxyl terminus of each AB2 is linked to the amino terminus each of the AB1 heavy chains; and
   ii. a second masking moiety (MM2) linked to a second cleavable moiety (CM2) to form a second prodomain, wherein the carboxyl terminus of the second prodomain is linked to the amino terminus of each AB2 wherein
      the MM2 inhibits the binding of the AB2 to its target; and
      the CM2 is a polypeptide that functions as a substrate for a second protease,
and wherein the BAA has the following characteristics:
i. MM2 comprises amino acid sequence SEQ ID NO: 105 or SEQ ID NO: 106SEQ ID NO: 107;
ii. MM1 comprises an amino acid sequence selected from the group consisting of sequences presented in Table 7;
iii. AB2 comprises a heavy chain variable domain as set forth in SEQ ID NO: 2 or SEQ ID NO: 3 or a light chain variable domain as set forth in SEQ ID NO: 1 or SEQ ID NO: 4; and
iv. AB1 comprises an Fc region comprising an amino acid substitution in at least one of amino acid positions L234, L235, N297, and P331 or L234, L235 and P331, as numbered by the EU index as set forth in Kabat, such that the BAA has reduced effector function.

In some embodiments, the BAAs provided herein comprise:
a) an IgG antibody (AB1) that specifically binds to a first target wherein the AB1 comprises:
   a. two heavy chains (AB1 HCs) and two light chains (AB1 LCs); and
   b. a first masking moiety (MM1) linked to a first cleavable moiety (CM1) to form a prodomain, wherein the carboxyl terminus of the prodomain is linked to each amino terminus of each light chain of the AB1, wherein
      1. the MM1 inhibits the binding of the AB1 to its target; and
      2. the CM1 is a polypeptide that functions as a substrate for a first protease,
b) two scFvs (AB2) that each specifically binds to a second target wherein each AB2 comprises:
   a. a heavy chain variable region linked to a light chain variable region, wherein the carboxyl terminus of each AB2 is linked to the amino terminus each of the AB1 heavy chains; and
   b. a second masking moiety (MM2) linked to a second cleavable moiety (CM2) to form a prodomain, wherein the carboxyl terminus of the prodomain is linked to the amino terminus of each AB2 wherein
      1. the MM2 inhibits the binding of the AB2 to its target; and
      2. the CM2 is a polypeptide that functions as a substrate for a second protease,
and wherein the AB1 comprises an Fc region comprises an amino acid substitution in at least one of amino acid positions L234, L235, N297, and P331, as numbered by the EU index as set forth in Kabat, such that the BAA has reduced effector function. In some embodiments, the Fc region comprises amino acid substitutions in at least two amino acid positions L234, L235, N297 and P331, as numbered by the EU index as set forth in Kabat, such that the BAA has reduced effector function.

In some embodiments, the Fc region comprises amino acid substitutions in at least amino acid positions L234, L235, and P331, as numbered by the EU index as set forth in Kabat, such that the BAA has reduced effector function. In some embodiments, the first target is selected from the group consisting of the targets presented in Table 9 and the second target is selected from the group consisting of the targets presented in Table 9.

In some embodiments, AB1 binds a target antigen, e.g. a tumor antigen, and the AB2 binds an immune effector target.

In some embodiments, AB2 binds a target antigen, e.g. a tumor antigen, and the AB1 binds an immune effector target.

In some embodiments, the AB1 binds EGFR and the AB2 binds CD3. In some embodiments, the AB1 is an EGFR-binding antibody, or fragment thereof, as disclosed herein, and the AB2 is a CD3-binding antibody, or fragment thereof, as disclosed herein.

In some embodiments, the MM1 comprises an amino acid sequence selected from SEQ ID NOs: 78-87. In some embodiments, the MM1 comprises SEQ ID NO: 78.

In some embodiments, the MM2 comprises an amino acid sequence selected from SEQ ID NOs: 12, 106, 107 and 105. In some embodiments, the MM2 comprises the amino acid sequence SEQ ID NO: 12.

In some embodiments, the bispecific AA is CI106, CI138, CI139, CI158 or CI140 as provided in Table 11, in Example 1.

In an exemplary embodiment, AB1 comprises the amino acid sequence of C225v5Fcmt3 HC or C225v5Fcmt4 HC.

In some embodiments, the first and second proteases are the same protease. In some embodiments, the first and second proteases are different proteases. In some embodiments, CM1 and CM2 comprise the same amino acid sequence. In some embodiments, CM1 and CM2 comprise different amino acid sequences. In some embodiments, CM1 and CM2 comprise different amino acid sequences that are cleavable by the same protease or proteases. In some embodiments, CM1 and CM2 are cleavable by more than one protease. In some embodiments, CM1 and/or CM2 is cleavable by a serine protease. In some embodiments, CM1 and/or CM2 is cleavable by a matrix metalloproteinase (MMP). In some embodiments, CM1 and/or CM2 is cleavable by a serine protease and an MMP.

Exemplary BAAs of the disclosure include, for example, those shown in the Examples provided herein, and variants thereof.

In some non-limiting embodiments, at least one of the AB in the BAA is specific for CD3 and at least one other AB is a binding partner for any target listed in Table 9.

In an exemplary embodiment, AB2 of the BAA is specific for CD3 and AB1 is a binding partner for any target listed in Table 9.

TABLE 9
Exemplary Targets

| | | | | | |
|---|---|---|---|---|---|
| 1-92-LFA-3 | CD52 | DL44 | HVEM | LAG-3 | STEAP1 |
| Alpha-4 integrin | CD56 | DLK1 | Hyaluronidase | LIF-R | STEAP2 |
| Alpha-V integrin | CD64 | DLL4 | ICOS | Lewis X | TAG-72 |
| alpha4beta1 integrin | CD70 | DPP-4 | IFNalpha | LIGHT | TAPA1 |
| alpha4beta7 integrin | CD71 | DSG1 | IFNbeta | LRP4 | TGFbeta |
| AGR2 | CD74 | EGFR | IFNgamma | LRRC26 | TIGIT |
| Anti-Lewis-Y | | EGFRviii | IgE | MCSP | TIM-3 |
| Apelin J receptor | CD80 | Endothelin B | IgE Receptor | Mesothelin | TLR2 |
| | | receptor (ETBR) | (FceRI) | | |
| APRIL | CD81 | ENPP3 | IGF | MRP4 | TLR4 |
| B7-H4 | CD86 | EpCAM | IGF1R | MUC1 | TLR6 |
| BAFF | CD95 | EPHA2 | IL1B | Mucin-16 | TLR7 |
| | | | | (MUC16, CA-125) | |
| BTLA | CD117 | EPHB2 | IL1R | Na/K ATPase | TLR8 |
| C5 complement | CD125 | ERBB3 | IL2 | Neutrophil elastase | TLR9 |
| C-242 | CD132 (IL-2RG) | F protein of RSV | IL11 | NGF | TMEM31 |
| CA9 | CD133 | FAP | IL12 | Nicastrin | TNFalpha |
| CA19-9 (Lewis a) | CD137 | FGF-2 | IL12p40 | Notch Receptors | TNFR |
| Carbonic anhydrase 9 | CD138 | FGF8 | IL-12R, IL-12Rbeta1 | Notch 1 | TNFRS12A |
| CD2 | CD166 | FGFR1 | IL13 | Notch 2 | TRAIL-R1 |
| CD3 | CD172A | FGFR2 | IL13R | Notch 3 | TRAIL-R2 |
| CD6 | CD248 | FGFR3 | IL15 | Notch 4 | Transferrin |
| CD9 | CDH6 | FGFR4 | IL17 | NOV | Transferrin receptor |
| CD11a | CEACAM5 (CEA) | Folate receptor | IL18 | OSM-R | TRK-A |
| CD19 | CEACAM6 (NCA-90) | GAL3ST1 | IL21 | OX-40 | TRK-B |
| CD20 | CLAUDIN-3 | G-CSF | IL23 | PAR2 | uPAR |
| CD22 | CLAUDIN-4 | G-CSFR | IL23R | PDGF-AA | VAP1 |
| CD24 | cMet | GD2 | IL27/IL27R (wsx1) | PDGF-BB | VCAM-1 |
| CD25 | Collagen | GITR | IL29 | PDGFRalpha | VEGF |
| CD27 | Cripto | GLUT1 | IL-31R | PDGFRbeta | VEGF-A |
| CD28 | CSFR | GLUT4 | IL31/IL31R | PD-1 | VEGF-B |
| CD30 | CSFR-1 | GM-CSF | IL2R | PD-L1 | VEGF-C |
| CD33 | CTLA-4 | GM-CSFR | IL4 | PD-L2 | VEGF-D |
| CD38 | CTGF | GP IIb/IIIa | IL4R | Phosphatidyl- | VEGFR1 |
| | | receptors | | serine | |
| CD40 | CXCL10 | Gp130 | IL6, IL6R | P1GF | VEGFR2 |
| CD40L | CXCL13 | GPIIB/IIIA | Insulin Receptor | PSCA | VEGFR3 |
| CD41 | CXCR1 | GPNMB | Jagged Ligands | PSMA | VISTA |
| CD44 | CXCR2 | GRP78 | Jagged 1 | RAAG12 | WISP-1 |
| CD44v6 | | HER2/neu | Jagged 2 | RAGE | WISP-2 |
| CD47 | CXCR4 | HGF | | SLC44A4 | WISP-3 |
| CD51 | CYR61 | hGH | | Sphingosine 1 | |
| | | | | Phosphate | |

In some embodiments, the unmasked EGFR-CD3 bispecific antibody exhibits EGFR-dependent tumor cell killing, while the doubly-masked EGFR-CD3 BAA reduces target-dependent cytotoxicity by more than 100,000-fold. In established tumor models where tumor-resident proteases are expected to be active, it is shown that BAAs potently induce tumor regressions. In non-human primates, the maximum tolerated dose (MTD) of the EGFR-CD3 BAA is more than 60-fold higher than the MTD of the unmasked bispecific antibody, and the tolerated exposure (AUC) is more than 10,000-fold higher. Despite the 60-fold dose differential at the MTDs, transient serum cytokine and AST/ALT increases observed in non-human primates treated with the BAA are still lower than those induced by the bispecific antibody. By localizing activity to the tumor microenvironment, BAAs have the potential to expand clinical opportunities for T cell-engaging bispecific therapies that are limited by on target toxicities, especially in solid tumors. Moreover, an EGFR-CD3 BAA has the potential to address EGFR-expressing tumors that are poorly responsive to existing EGFR-directed therapies.

### 7. Cleavable Moieties (CM)

Both the monospecific AAs and the BAAs of the disclosure comprise at least one CM, when masked and not activated.

In some embodiments, the cleavable moiety (CM) of the AA or BAA includes an amino acid sequence that can serve as a substrate for at least one protease, usually an extracellular protease. In the case of an AA or BAA, the CM may be selected based on a protease that is co-localized in tissue with the desired target of at least one AB of the BAA or AB of the AA. A CM can serve as a substrate for multiple proteases, e.g., a substrate for a serine protease and a second different protease, e.g. an MMP). In some embodiments, a CM can serve as a substrate for more than one serine protease, e.g., a matriptase and a uPA. In some embodiments, a CM can serve as a substrate for more than one MMP, e.g., an MMP9 and an MMP14.

A variety of different conditions are known in which a target of interest is co-localized with a protease, where the substrate of the protease is known in the art. In the example of cancer, the target tissue can be a cancerous tissue, particularly cancerous tissue of a solid tumor. There are reports in the literature of increased levels of proteases in a number of cancers, e.g., liquid tumors or solid tumors. See, e.g., La Rocca et al, (2004) British J. of Cancer 90(7): 1414-1421. Non-limiting examples of disease include: all types of cancers, (such as, but not limited to breast, lung, colorectal, gastric, glioblastoma, ovarian, endometrial, renal, sarcoma, skin cancer, cervical, liver, bladder, cholangiocarcinoma, prostate, melanomas, head and neck cancer (e.g. head and neck squamous cell cancer, pancreatic, etc.), rheumatoid arthritis, Crohn's disease, SLE, cardiovascular damage, ischemia, etc. For example, indications would include leukemias, including T-cell acute lymphoblastic leukemia (T-ALL), lymphoblastic diseases including multiple myeloma, and solid tumors, including lung, colorectal, prostate, pancreatic and breast, including triple negative breast cancer. For example, indications include bone disease or metastasis in cancer, regardless of primary tumor origin; breast cancer, including by way of non-limiting example, ER/PR+ breast cancer, Her2+ breast cancer, triple-negative breast cancer; colorectal cancer; endometrial cancer; gastric cancer; glioblastoma; head and neck cancer, such as head and neck squamous cell cancer; esophageal cancer; lung cancer, such as by way of non-limiting example, non-small cell lung cancer; multiple myeloma ovarian cancer; pancreatic cancer; prostate cancer; sarcoma, such as osteosarcoma; renal cancer, such as by way of non-limiting example, renal cell carcinoma; and/or skin cancer, such as by way of non-limiting example, squamous cell cancer, basal cell carcinoma, or melanoma

The CM is specifically cleaved by an enzyme at a rate of about 0.001-1500×10⁴ M⁻¹ S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹ S⁻¹.

For specific cleavage by an enzyme, contact between the enzyme and CM is made. When the AA or BAA comprises at least a first AB coupled to a MM and a CM, e.g., the AA or BAA comprises an AB coupled to a MM via a CM, is in the presence of target and sufficient enzyme activity, the CM can be cleaved. Sufficient enzyme activity can refer to the ability of the enzyme to make contact with the CM and effect cleavage. It can readily be envisioned that an enzyme may be in the vicinity of the CM but is unable to cleave because of other cellular factors or protein modification of the enzyme.

Exemplary CMs of the disclosure are provided in Table 4 above. In some embodiments, the CM has a length of up to 15 amino acids, a length of up to 20 amino acids, a length of up to 25 amino acids, a length of up to 30 amino acids, a length of up to 35 amino acids, a length of up to 40 amino acids, a length of up to 45 amino acids, a length of up to 50 amino acids, a length of up to 60 amino acids, a length in the range of 10-60 amino acids, a length in the range of 15-60 amino acids, a length in the range of 20-60 amino acids, a length in the range of 25-60 amino acids, a length in the range of 30-60 amino acids, a length in the range of 35-60 amino acids, a length in the range of 40-50 amino acids, a length in the range of 45-60 amino acids, a length in the range of 10-40 amino acids, a length in the range of 15-40 amino acids, a length in the range of 20-40 amino acids, a length in the range of 25-40 amino acids, a length in the range of 30-40 amino acids, a length in the range of 35-40 amino acids, a length in the range of 10-30 amino acids, a length in the range of 15-30 amino acids, a length in the range of 20-30 amino acids, a length in the range of 25-30 amino acids, a length in the range of 10-20 amino acids, or a length in the range of 10-15 amino acids.

### 8. Masking Moieties (MMs)

As described herein, the AAs and BAAs of the invention comprise a prodomain, which comprises a MM.

In some embodiments, the MM is selected for use with a specific antibody or antibody fragment.

In certain embodiments, the MM is not a natural binding partner of the AB. In some embodiments, the MM contains no or substantially no homology to any natural binding partner of the AB. In some embodiments the MM is no more than 5%, 10%, 15%, 20%, 25%, 30%, 35%, 40%, 45%, 50%, 55%, 60%, 65%, 70%, 75%, or 80% similar to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% identical to any natural binding partner of the AB. In some embodiments, the MM is no more than 50% identical to any natural binding partner of the AB. In some embodiments, the MM is no more than 25% identical to any natural binding partner of the AB. In some embodiments, the MM is no more than 20% identical to any natural binding partner of the AB. In some embodiments, the MM is no more than 10% identical to any natural binding partner of the AB.

Exemplary MMs of the disclosure can have a length of up to 15 amino acids, a length of up to 20 amino acids, a length of up to 25 amino acids, a length of up to 30 amino acids, a length of up to 35 amino acids, a length of up to 40 amino acids, a length of up to 45 amino acids, a length of up to 50 amino acids, a length of up to 60 amino acids, a length in the range of 10-60 amino acids, a length in the range of 15-60 amino acids, a length in the range of 20-60 amino acids, a length in the range of 25-60 amino acids, a length in the range of 30-60 amino acids, a length in the range of 35-60 amino acids, a length in the range of 40-50 amino acids, a length in the range of 45-60 amino acids, a length in the range of 10-40 amino acids, a length in the range of 15-40 amino acids, a length in the range of 20-40 amino acids, a length in the range of 25-40 amino acids, a length in the range of 30-40 amino acids, a length in the range of 35-40 amino acids, a length in the range of 10-30 amino acids, a length in the range of 15-30 amino acids, a length in the range of 20-30 amino acids, a length in the range of 25-30 amino acids, a length in the range of 10-20 amino acids, a length in the range of 10-15 amino acids, or a length of 10, 11, 12, 13, 14, 15, 16, 17, 18, 19, or 20 amino acids.

Exemplary MMs of the disclosure are provided in Tables 3, 7, and 8, above.

### 9. Linkers

In many embodiments, it may be desirable to insert one or more linkers, e.g., flexible linkers, into the AA/BAA constructs so as to provide for flexibility at one or more of the MM-CM junction, the CM-AB/CM-scFv junction, or both. For example, the AB, MM, and/or CM may not contain a sufficient number of residues (e.g., Gly, Ser, Asp, Asn, especially Gly and Ser) to provide the desired flexibility. As such, the ability of such BAA constructs to remain intact or be activated as disclosed herein may benefit from introduction of one or more amino acids to provide for a flexible linker.

For example, in certain embodiments an AA comprises one of the following formulae (where the formula below represents an amino acid sequence in either N- to C-terminal direction or C- to N-terminal direction):
(MM1)-L1-(CM1)-(AB1)
(MM1)-(CM1)-L2-(AB1)
(MM1)-L1-(CM1)-L2-(AB1)
(MM2)-L1-(CM2)-(AB2)
(MM2)-(CM2)-L2-(AB2)
(MM2)-L1-(CM2)-L2-(AB2)
   wherein MM, CM, and AB are as defined above; wherein L1 and L2 are each independently and optionally present or absent, are the same or different flexible linkers that include at least 1 flexible amino acid (e.g., Gly, Ser).

In some embodiments, the BAA comprises 2 heavy chains, each comprising the structural arrangement from N-terminus to C-terminus of MM2-CM2-AB2-AB1 HC and two light chains each comprising the structural arrangement from N-terminus to C-terminus of MM1-CM1-AB1 LC.

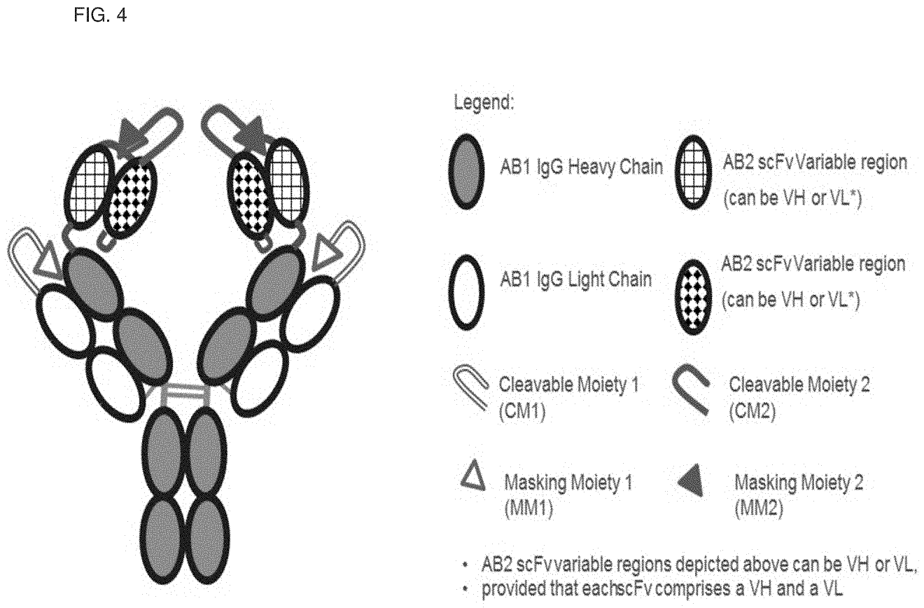

In some embodiments, the structure including with linkers is provided in FIG. 4.

In some embodiments, (MM2)-L1-(CM2)-L2-(AB2) is linked to the heavy chain of AB1 and AB2 is a scFv.

Linkers suitable for use in compositions described herein are generally ones that provide flexibility of the modified AB or the AAs to facilitate the inhibition of the binding of the AB to the target. Such linkers are generally referred to as flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, 7, 8, 9, 10, 11, 12, 13, 14, 15, 16, 17, 18, 19, or 20 amino acids in length. In some embodiments, a suitable linker can be from 4 to 25 amino acids in length. In some embodiments, a suitable linker can be from 5 to 25 amino acids in length. In some embodiments, a suitable linker can be from 4 to 20 amino acids in length. In some embodiments, a suitable linker can be from 5 to 20 amino acids in length.

Exemplary linkers include glycine polymers (G)n, glycine-serine polymers (including, for example, (GS)n, (GSGGS)n (SEQ ID NO: 88) and (GGGS)n (SEQ ID NO: 89), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). Exemplary linkers are provided in Table 9-1.

TABLE 9-1
Exemplary L1 and L2 Linkers

| SEQ ID NO: | Linker Amino Acid Sequence |
|---|---|
| SEQ ID NO: 88 | GSGGS |
| SEQ ID NO: 89 | GGGS |
| SEQ ID NO: 90 | GGSG |
| SEQ ID NO: 91 | GGSGG |
| SEQ ID NO: 92 | GSGSG |
| SEQ ID NO: 93 | GSGGG |
| SEQ ID NO: 94 | GGGSG |
| SEQ ID NO: 95 | GSSSG |
| SEQ ID NO: 96 | GSSGGSGGSGG |
| SEQ ID NO: 97 | GGGS |
| SEQ ID NO: 99 | GGGGS |
| SEQ ID NO: 100 | GSSGGSGGSGGSG |
| SEQ ID NO: 101 | GSSGGSGGSGGGGGSGGGSGGGS |
| SEQ ID NO: 102 | GSSGGSGGSGGSGGGSGGGSGGS |
| SEQ ID NO: 103 | GSSGT |
| SEQ ID NO: 104 | GGGSSGGS |

The ordinarily skilled artisan will recognize that design of an AA can include linkers that are all or partially flexible, such that the linker can include a flexible linker as well as one or more portions that confer less flexible structure to provide for a desired AA structure.

### 10. Conjugation

In some embodiments, the antibodies, or ABs of the AAs, and BAAs are conjugated to an agent. In some embodiments, the agent is a therapeutic agent. In some embodiments, the agent is a detectable moiety. In some embodiments, the agent is an antineoplastic agent. In some embodiments, the agent is a toxin or fragment thereof. In some embodiments, the agent is conjugated to the AB via a linker. In some embodiments, the linker is a non-cleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the linker is a cleavable linker. In some embodiments, the agent is an agent selected from the group listed in Table 10.

TABLE 10
Exemplary Pharmaceutical Agents for Conjugation

CYTOTOXIC AGENTS
Auristatins
Auristatin E
Monomethyl auristatin D (MMAD)
Monomethyl auristatin E (MMAE)
Desmethyl auristatin E (DMAE)
Auristatin F
Monomethyl auristatin F (MMAF)
Desmethyl auristatin F (DMAF)
Auristatin derivatives, e.g., amides thereof
Auristatin tyramine
Auristatin quinoline
Dolastatins
Dolastatin derivatives
Dolastatin 16 DmJ
Dolastatin 16 Dpv
Maytansinoids, e.g. DM-1; DM-4
Maytansinoid derivatives
Duocarmycin
Duocarmycin derivatives
Alpha-amanitin
Anthracyclines
Doxorubicin
Daunorubicin
Bryostatins
Camptothecin
Camptothecin derivatives
7-substituted Camptothecin
10,11-Difluoromethylenedioxycamptothecin
Combretastatins
Debromoaplysiatoxin
Kahalalide-F
Discodermolide
Ecteinascidins
ANTIVIRALS
Acyclovir
Vira A
Symmetrel
ANTIFUNGALS
Nystatin
ADDITIONAL ANTI-NEOPLASTICS
Adriamycin
Cerubidine
Bleomycin
Alkeran
Velban
Oncovin
Fluorouracil
Methotrexate
Thiotepa
Bisantrene
Novantrone
Thioguanine
Procarabizine
Cytarabine
ANTI-BACTERIALS
Aminoglycosides
Streptomycin
Neomycin
Kanamycin
Amikacin
Gentamicin
Tobramycin
Streptomycin B
Spectinomycin
Ampicillin
Sulfanilamide
Polymyxin
Chloramphenicol
Turbostatin
Phenstatins
Hydroxyphenstatin
Spongistatin 5
Spongistatin 7
Halistatin 1
Halistatin 2
Halistatin 3
Modified Bryostatins
Halocomstatins
Pyrrolobenzimidazoles
Cibrostatin6
Doxaliform
Anthracyclins analogues
Cemadotin analogue (CemCH2-SH)
Pseudomonas toxin A (PE38) variant
Pseudomonas toxin A (ZZ-PE38) variant
ZJ-101
OSW-1
4-Nitrobenzyloxycarbonyl Derivatives of
O6-Benzylguanine
Topoisomerase inhibitors
Hemiasterlin
Cephalotaxine
Homoharringtonine
Pyrrolobenzodiazepine dimers (PBDs)
Functionalized pyrrolobenzodiazepenes
Calicheamicins
Podophyllotoxins
Taxanes
Vinca alkaloids
CONJUGATABLE DETECTABLE MOIETIES
Fluorescein and derivatives thereof
Fluorescein isothiocyanate (FITC)
RADIOPHARMACEUTICALS
¹²⁵I
¹³¹I
⁸⁹Zr
¹¹¹In
¹²³I
¹³¹I
⁹⁹mTc
²⁰¹Tl
¹³³Xe
¹¹C
⁶²Cu
¹⁸F
⁶⁸Ga
¹³N
¹⁵O
³⁸K
⁸²Rb
⁹⁹mTc (Technetium)
HEAVY METALS
Barium
Gold
Platinum
ANTI-MYCOPLASMALS
Tylosine
Spectinomycin

Those of ordinary skill in the art will recognize that a large variety of possible moieties can be coupled to the resultant antibodies, AAs, and BAAs of the disclosure. (See, for example, “Conjugate Vaccines”, Contributions to Microbiology and Immunology, J. M. Cruse and R. E. Lewis, Jr (eds), Karger Press, New York, (1989), the entire contents of which are incorporated herein by reference).

In some embodiments, the antibody, AA or BAA comprises a detectable moiety. In some embodiments, the detectable moiety is a diagnostic agent.

In some embodiments, the antibody, AA or BAA contains one or more disulfide bonds. In some embodiments, the antibody, AA or BAA contains one or more lysines. In some embodiments, the antibody, AA or BAA can be engineered to include one or more disulfide bonds or can be otherwise engineered to enable site-specific conjugation.

### 11. Production

The disclosure also provides an isolated nucleic acid molecule encoding an antibody, AA or BAA described herein, as well as vectors that include these isolated nucleic acid sequences. The disclosure provides methods of producing an antibody, AA or BAA by culturing a cell under conditions that lead to expression of the antibody, AA or BAA, wherein the cell comprises such a nucleic acid molecule.

In some embodiments, the cell comprises such a vector. In some embodiments, the vector is pLW307. In some embodiments, the vector is pLW291. In some embodiments, the vector is pEF1049. In some embodiments, the vector is pEF1050. In some embodiments, the vector is pEF1107. In some embodiments, the vector is pEF1052. (these vectors are described and sequences provided below in Example 1)

### 12. Use of Antibodies, AAs, Bispecific Antibodies and BAAs

In some embodiments, the antibodies/bispecific antibodies/AAs/BAAs thereof may be used as therapeutic agents. Such agents will generally be employed to treat, alleviate, and/or prevent a disease or pathology in a subject. A therapeutic regimen is carried out by identifying a subject, e.g., a human patient or other mammal suffering from (or at risk of developing) a disorder using standard methods.

Administration of the antibodies/bispecific antibodies/AAs/BAAs thereof may abrogate or inhibit or interfere with the signaling function of one or more of the targets.

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Generally, alleviation or treatment of a disease or disorder involves the lessening of one or more symptoms or medical problems associated with the disease or disorder. For example, in the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. In some embodiments, a composition of this disclosure can be used to prevent the onset or reoccurrence of the disease or disorder in a subject, e.g., a human or other mammal, such as a non-human primate, companion animal (e.g., cat, dog, horse), farm animal, work animal, or zoo animal. The terms subject and patient are used interchangeably herein.

A therapeutically effective amount of antibodies/bispecific antibodies/AAs/BAAs thereof of the disclosure relates generally to the amount needed to achieve a therapeutic objective.

Common ranges for therapeutically effective dosing of an antibodies/bispecific antibodies/AAs/BAAs thereof of the disclosure may be, by way of nonlimiting example, from about 0.1 mg/kg body weight to about 50 mg/kg body weight. Common dosing frequencies may range, for example, from twice daily to once a week.

Efficaciousness of treatment is determined in association with any known method for diagnosing or treating the particular disorder. Methods for the screening antibodies/bispecific antibodies/AAs/BAAs that possess the desired specificity include, but are not limited to, enzyme linked immunosorbent assay (ELISA) and other immunologically mediated techniques known within the art.

Other contemplated uses involve diagnostics, imaging, prognostics, and detection uses. In some embodiments, antibodies/bispecific antibodies/AAs/BAAs are used in methods known within the art relating to the localization and/or quantitation of the target (e.g., for use in measuring levels of one or more of the targets within appropriate physiological samples, for use in diagnostic methods, for use in imaging the protein, and the like).

In some embodiments, antibodies/bispecific antibodies/AAs/BAAs are used to isolate one or more of the targets by standard techniques, such as immunoaffinity, chromatography or immunoprecipitation. An antibody, an AA, a bispecific antibody or a BAA can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In yet another embodiment, an antibody, bispecific antibody, AA, BAA directed two or more targets can be used as an agent for detecting the presence of one or more of the targets (or a fragment thereof) in a sample. In some embodiments, the antibody contains a detectable label. Antibodies are polyclonal, or in some embodiments, monoclonal. An intact antibody, or a fragment thereof (e.g., Fab, scFv, or F(ab′)₂) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of an antibody with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the disclosure can be used to detect a protein in a biological sample in vitro as well as in vivo. For example, in vitro techniques for detection of an analyte protein include enzyme linked immunosorbent assays (ELISAs), Western blots, immunoprecipitations, and immunofluorescence. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

The antibodies, bispecific antibodies, AAs, and bispecific antibodies of the disclosure are also useful in a variety of diagnostic and prophylactic formulations. In one embodiment, an antibody, AA, bispecific antibody, BAA is administered to patients that are at risk of developing one or more of the aforementioned disorders. A patient's or organ's predisposition to one or more of the disorders can be determined using genotypic, serological or biochemical markers.

In another embodiment of the disclosure, an antibody, AA, bispecific antibody, BAA is administered to human individuals diagnosed with a clinical indication associated with one or more of the aforementioned disorders. Upon diagnosis, an antibody, AA, bispecific antibody, BAA is administered to mitigate or reverse the effects of the clinical indication.

Antibodies, bispecific antibodies, AAs, and bispecific antibodies are also useful in the detection of one or more targets in patient samples and accordingly are useful as diagnostics. For example, the antibodies, bispecific antibodies, AAs, and bispecific antibodies of the disclosure are used in in vitro assays, e.g., ELISA, to detect one or more target levels in a patient sample.

In one embodiment, an antibody, AA, bispecific antibody, BAA is immobilized on a solid support (e.g., the well(s) of a microtiter plate). The immobilized antibody and/or AA serves as a capture antibody for any target(s) that may be present in a test sample. Prior to contacting the immobilized antibody/AA with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen(s) in the test sample is determined by comparison with a standard curve developed from the standard samples.

It will be appreciated that based on the results obtained using the antibody, AA, bispecific antibody, BAA in an in vitro diagnostic assay, it is possible to stage a disease in a subject based on expression levels of the target antigen(s). For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

Antibodies, bispecific antibodies, AAs, and BAAs can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, AAs, and bispecific antibodies having an enzymatically cleavable CM can be used to detect the presence or absence of an enzyme that is capable of cleaving the CM. Such AAs, and bispecific antibodies can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated or bispecific activated antibodies (i.e., antibodies or bispecific antibodies resulting from cleavage of an AA or a BAA) in a given cell or tissue of a given host organism. Such accumulation of activated bispecific antibodies indicates not only that the tissue expresses enzymatic activity (or an increased reduction potential depending on the nature of the CM) but also that the tissue expresses at least one target to which the activated bispecific antibody binds.

For example, the CM can be selected to be a protease substrate for a protease found at the site of a tumor, at the site of a viral or bacterial infection at a biologically confined site (e.g., such as in an abscess, in an organ, and the like), and the like. At least one of the AB can be one that binds a target antigen. Using methods familiar to one skilled in the art, a detectable label (e.g., a fluorescent label or radioactive label or radiotracer) can be conjugated to an AB or other region of an antibody, AA, bispecific antibody, BAA. Suitable detectable labels are discussed in the context of the above screening methods and additional specific examples are provided below. Using at least one AB specific to a protein or peptide of the disease state, along with a protease whose activity is elevated in the disease tissue of interest, AAs will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated bispecific antibodies in the disease tissue is enhanced relative to non-disease tissues.

In another example, antibodies, antibodies/bispecific antibodies/AAs/BAAs of the present disclosure can be used to detect the presence or absence of a cleaving agent in a sample. For example, where the antibodies/bispecific antibodies/AAs/BAAs contain a CM susceptible to cleavage by an enzyme, the BAAs can be used to detect (either qualitatively or quantitatively) the presence of an enzyme in the sample. In another example, where the antibodies/bispecific antibodies/AAs/BAAs contain a CM susceptible to cleavage by reducing agent, the antibodies/bispecific antibodies/AAs/BAAs can be used to detect (either qualitatively or quantitatively) the presence of reducing conditions in a sample. To facilitate analysis in these methods, the antibodies/bispecific antibodies/AAs/BAAs can be detectably labeled, and can be bound to a support (e.g., a solid support, such as a slide or bead). The detectable label can be positioned on a portion of the antibodies/bispecific antibodies/AAs/BAAs that is not released following cleavage, for example, the detectable label can be a quenched fluorescent label or other label that is not detectable until cleavage has occurred. The assay can be conducted by, for example, contacting the immobilized, detectably labeled antibodies/bispecific antibodies/AAs/BAAs with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the antibodies/bispecific antibodies/AAs/BAAs prior to contacting with the sample e.g., the presence of and/or an increase in detectable signal due to cleavage of the antibodies/bispecific antibodies/AAs/BAAs by the cleaving agent in the sample.

Such detection methods can be adapted to also provide for detection of the presence or absence of a target that is capable of binding at least one AB of the antibodies/bispecific antibodies/AAs/BAAs of the present disclosure. Thus, the assays can be adapted to assess the presence or absence of a cleaving agent and the presence or absence of a target of interest. The presence or absence of the cleaving agent can be detected by the presence of and/or an increase in detectable label of the antibodies/bispecific antibodies/AAs/BAAs as described above, and the presence or absence of the target can be detected by detection of a target-AB complex e.g., by use of a detectably labeled anti-target antibody.

AAs/BAAs of the present disclosure are also useful in in situ imaging for the validation of AA activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an AA/BAA is labeled with a detectable label. The detectable label may be a fluorescent dye, (e.g. a fluorophore, Fluorescein Isothiocyanate (FITC), Rhodamine Isothiocyanate (TRITC), an Alexa Fluor® label), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, AA or BAA indicates that the sample contains the target and contains a protease that is specific for the CM of the AAs or BAAs of the present disclosure. In some embodiments, the presence of the protease can be confirmed using broad spectrum protease inhibitors such as those described herein, and/or by using an agent that is specific for the protease, for example, an antibody such as A11, which is specific for the protease matriptase (MT-SP1) and inhibits the proteolytic activity of MT-SP1; see e.g., International Publication Number WO 2010/129609, published 11 Nov. 2010. The same approach of using broad spectrum protease inhibitors such as those described herein, and/or by using a more selective inhibitory agent can be used to identify a protease or class of proteases specific for the CM of the AAs or BAAs of the present disclosure. In some embodiments, the presence of the target can be confirmed using an agent that is specific for the target or the detectable label can be competed with unlabeled target. In some embodiments, unlabeled AA could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the AAs or BAAs of the present disclosure.

These techniques are also useful in kits and/or as reagents for the detection, identification or characterization of protease activity in a variety of cells, tissues, and organisms based on the protease-specific CM in the AAs or BAAs of the present disclosure.

### 13. Therapeutic Administration

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

In some embodiments, the antibodies, bispecific antibodies, AAs, or BAAs (or conjugated compositions thereof) are administered in conjunction with one or more additional agents, or with a combination of additional agents. Suitable additional agents include current pharmaceutical and/or surgical therapies for an intended application. For example, they can be used in conjunction with an additional chemotherapeutic or antineoplastic agent.

In some embodiments, the antibodies, bispecific antibodies, AAs, or BAAs (or conjugated compositions thereof) of the present disclosure are administered in conjunction with one or more additional agents selected from the group consisting of antibodies, conjugated antibodies, AAs, conjugated AAs, bispecific antibodies, conjugated bispecific antibodies, BAAs, or conjugated BAAs. In some embodiments, the antibody portion of any of the above-referenced additional agents is directed against a target such as one or more of the targets disclosed in Table 9. It is appreciated that in some embodiments the antibody portion of antibodies, bispecific antibodies, AAs, or BAAs (or conjugated compositions thereof) of the present disclosure and the antibody portion of the additional agent is directed against the same target (e.g. both may target EGFR). In some embodiments, they are directed against the same target, but target different epitopes. In some embodiments, they are directed against different targets entirely (e.g., an activatable antibody of the present disclosure that targets EGFR may be administered in conjunction with an AA targeting a different target; likewise e.g. a BAA of the present disclosure that targets EGFR and CD3 may be administered in conjunction with an AA targeting a different target.

In some embodiments, antibodies, bispecific antibodies, AAs or BAAs (or conjugated compositions thereof) of the disclosure are administered in conjunction with an immunotherapeutic agent. In some embodiments, antibodies, bispecific antibodies, AAs or BAAs (or conjugated compositions thereof) of the disclosure are administered in conjunction with a chemotherapeutic agent. In some embodiments, antibodies, bispecific antibodies, AAs or BAAs (or conjugated compositions thereof) of the disclosure are administered in conjunction with both an immunotherapeutic agent and a chemotherapeutic agent. In some embodiments, one or more additional agents is administered with any of these combination embodiments.

In some embodiments, they are formulated into a single therapeutic composition, and the antibodies/bispecific antibodies/AAs/BAAs thereof and the additional agent are administered simultaneously. Alternatively, the antibodies/bispecific antibodies/AAs/BAAs thereof are administered separate from each other, e.g., each is formulated into a separate therapeutic composition, and the antibodies/bispecific antibodies/AAs/BAAs thereof and the additional agent are administered simultaneously, or the antibodies/bispecific antibodies/AAs/BAAs thereof and the additional agent are administered at different times during a treatment regimen. The antibodies/bispecific antibodies/AAs/BAAs thereof and the additional agent can be administered in multiple doses.

The antibodies/bispecific antibodies/AAs/BAAs thereof can be incorporated into pharmaceutical compositions suitable for administration. Principles and considerations involved in preparing such compositions, as well as guidance in the choice of components are provided, for example, in Remington's Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Such compositions typically comprise the antibodies/bispecific antibodies/AAs/BAAs thereof and a pharmaceutically acceptable carrier.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The formulations to be used for in vivo administration must be sterile. This is readily accomplished by filtration through sterile filtration membranes.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be suitable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as sustained/controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

For example, the active ingredients can be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The materials can also be obtained commercially from Alza Corporation and Nova Pharmaceuticals, Inc. Liposomal suspensions (including liposomes targeted to infected cells with monoclonal antibodies to viral antigens) and can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The formulation can also contain more than one active compound as necessary for the particular indication being treated, for example, those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, the active compounds are administered in combination therapy, i.e., combined with other agents, e.g., therapeutic agents, that are useful for treating pathological conditions or disorders, such as autoimmune disorders and inflammatory diseases. The term “in combination” in this context means that the agents are given substantially contemporaneously, either simultaneously or sequentially. If given sequentially, at the onset of administration of the second compound, the first of the two compounds is still detectable at effective concentrations at the site of treatment.

For example, the combination therapy can include one or more antibodies/bispecific antibodies/AAs/BAAs thereof of the disclosure coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Furthermore, one or more antibodies/bispecific antibodies/AAs/BAAs thereof described herein may be used in combination with two or more of the therapeutic agents described herein (e.g. one BAA administered with another BAA or AA of the disclosure, and the like). Such combination therapies may advantageously utilize lower dosages of the administered therapeutic agents, thus avoiding possible toxicities or complications associated with the various monotherapies.

In other embodiments, one or more antibodies of the disclosure can be coformulated with, and/or coadministered with, one or more anti-inflammatory drugs, immunosuppressants, or metabolic or enzymatic inhibitors. Nonlimiting examples of the drugs or inhibitors that can be used in combination with the antibodies described herein, include, but are not limited to, one or more of: nonsteroidal anti-inflammatory drug(s) (NSAIDs), e.g., ibuprofen, tenidap, naproxen, meloxicam, piroxicam, diclofenac, and indomethacin; sulfasalazine; corticosteroids such. as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors. Suitable therapeutic agents for use in combination with the antibodies of the disclosure include NSAIDs, CSAIDs, (DHODH) inhibitors (e.g., leflunomide), and folate antagonists (e.g., methotrexate).

Examples of additional inhibitors include one or more of: corticosteroids (oral, inhaled and local injection); immunosuppressants, e.g., cyclosporin, tacrolimus (FK-506); and mTOR inhibitors, e.g., sirolimus (rapamycin—RAPAMUNE™ or rapamycin derivatives, e.g., soluble rapamycin derivatives (e.g., ester rapamycin derivatives, e.g., CCI-779); agents that interfere with signaling by proinflammatory cytokines such as TNFα or IL-1 (e.g. IRAK, NIK, IKK, p38 or MAP kinase inhibitors); COX2 inhibitors, e.g., celecoxib, rofecoxib, and variants thereof; phosphodiesterase inhibitors, e.g., R973401 (phosphodiesterase Type IV inhibitor); phospholipase inhibitors, e.g., inhibitors of cytosolic phospholipase 2 (cPLA2) (e.g., trifluoromethyl ketone analogs); inhibitors of vascular endothelial cell growth factor or growth factor receptor, e.g., VEGF inhibitor and/or VEGF-R inhibitor; and inhibitors of angiogenesis. Suitable therapeutic agents for use in combination with the antibodies of the disclosure are immunosuppressants, e.g., cyclosporin, tacrolimus (FK-506); mTOR inhibitors, e.g., sirolimus (rapamycin) or rapamycin derivatives, e.g., soluble rapamycin derivatives (e.g., ester rapamycin derivatives, e.g., CCI-779); COX2 inhibitors, e.g., celecoxib and variants thereof; and phospholipase inhibitors, e.g., inhibitors of cytosolic phospholipase 2 (cPLA2), e.g., trifluoromethyl ketone analogs.

Additional examples of therapeutic agents that can be combined with an antibody of the disclosure include one or more of: 6-mercaptopurines (6-MP); azathioprine sulphasalazine; mesalazine; olsalazine; chloroquine/hydroxychloroquine (PLAQUENIL®); pencillamine; aurothiornalate (intramuscular and oral); azathioprine; coichicine; beta-2 adrenoreceptor agonists (salbutamol, terbutaline, salmeteral); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

In some embodiments, antibodies/bispecific antibodies/AAs/BAAs thereof of the disclosure can be combined with one or more antibodies/bispecific antibodies/AAs/BAAs thereof.

### 14. Kits

Provided herein are kits comprising any one or more of the antibodies, AAs, bispecific antibodies, and BAAs provided herein.

The kit may comprise any one or more of the antibodies, AAs, bispecific antibodies, and BAAs provided herein in a composition suitable for storage or shipping. The kit may comprise a vessel, a diluent, a solvent, a second composition, or any component suitable for converting a storage or shipping composition in to a composition suitable for use in a method disclosed herein; the method may be, for instance, a therapeutic method disclosed herein. The kit may comprise instructions for use.

### Illustrative Embodiments

The invention may be defined by reference to the following illustrative embodiments:

Embodiment 1. A bispecific activatable antibody (BAA), wherein said BAA, when activated, specifically binds to two targets, and wherein said BAA, when not activated, comprises the following structure:

a) an IgG antibody (AB1) that specifically binds to a first target wherein the AB1 comprises:

i. two heavy chains (AB1 HCs) and two light chains (AB1 LCs); and

ii. two first prodomains, each comprising a first masking moiety (MM1) linked to a first cleavable moiety (CM1) in the N-terminal to C-terminal direction, wherein the carboxyl terminus of each first prodomain is linked to the amino terminus of each light chain of the AB1, wherein
the MM1 reduces the binding of the AB1 to its target; and
the CM1 is a polypeptide that functions as a substrate for a first protease,
   b) two scFvs (AB2) that each specifically binds CD3_{ε}, wherein each AB2 comprises:

i. a light chain variable region (VL) linked to a heavy chain variable region (VH), wherein the carboxyl terminus of each AB2 is linked to the amino terminus of each of the AB1 heavy chains, wherein
the VL comprises an amino acid sequence as set forth in SEQ ID NO: 1 or SEQ ID NO:4 and the VH comprises an amino acid sequence as set forth in SEQ ID NO: 2 or SEQ ID NO: 3; and
the VL is linked to the VH by a linker L3 comprising an amino acid sequence selected from the group consisting of SEQ ID NO: 98 and SEQ ID NO: 108; and

ii. two second prodomains, each comprising a second masking moiety (MM2) linked to a second cleavable moiety (CM2) in the N-terminal to C-terminal direction, wherein the carboxyl terminus of each second prodomain is linked to the amino terminus of each AB2 wherein
the MM2 reduces the binding of the AB2 to CD3_{ε};
the MM2 comprises an amino acid sequence selected from the group consisting of SEQ ID NO: 12, SEQ ID NO: 105, SEQ ID NO: 106 and SEQ ID NO: 107; and
the CM2 is a polypeptide that functions as a substrate for a second protease.

Embodiment 2. The BAA of embodiment 1, wherein the VL is linked to the VH by a linker comprising amino acid sequence SEQ ID NO: 98.

Embodiment 3. The BAA of embodiment 1, wherein the VL is linked to the VH by a linker comprising amino acid sequence SEQ ID NO: 108.

Embodiment 4. The BAA of any one of embodiments 1 to 3, wherein the MM2 comprises amino acid sequence SEQ ID NO: 12.

Embodiment 5. The BAA of any one of embodiments 1 to 3, wherein the MM2 comprises amino acid sequence SEQ ID NO: 105.

Embodiment 6. The BAA of any one of embodiments 1 to 3, wherein the MM2 comprises amino acid sequence SEQ ID NO: 106.

Embodiment 7. The BAA of any one of embodiments 1 to 3, wherein the MM2 comprises amino acid sequence SEQ ID NO: 107.

Embodiment 8. The BAA of any one of embodiments 1 to 7, wherein the AB1 binds a tumor target.

Embodiment 9. The BAA of any one of embodiments 1 to 8, wherein the AB1 binds EGFR.

Embodiment 10. The BAA of any one of embodiments 1 to 9, wherein the MM1 comprises an amino acid sequence selected from the group consisting of sequences presented in Table 7 or Table 8.

Embodiment 11. The BAA of any one of embodiments 1 to 10, wherein the MM1 comprises an amino acid sequence selected from the group consisting of SEQ ID NO: 78 and SEQ ID NO: 85.

Embodiment 12. The BAA of any one of embodiments 1 to 11, wherein the MM1 comprises amino acid sequence SEQ ID NO: 78.

Embodiment 13. The BAA of any one of embodiments 1 to 11, wherein the MM1 comprises amino acid sequence SEQ ID NO: 85.

Embodiment 14. The BAA of any one of embodiments 1 to 13, wherein the CM1 or CM2 comprises the amino acid sequence of any one of the CMs listed in Table 4 or Table 4-1.

Embodiment 15. The BAA of any one of embodiments 1 to 14, wherein the CM1 or CM2 comprises the amino acid sequence of SEQ ID NO: 14, SEQ ID NO: 16, or SEQ ID NO: 17.

Embodiment 16. The BAA of any one of embodiments 1 to 15, wherein the CM1 comprises an amino acid sequence selected from the group consisting of SEQ ID NO: 14 and SEQ ID NO: 16.

Embodiment 17. The BAA of any one of embodiments 1 to 15, wherein the CM2 comprises an amino acid sequence selected from the group consisting of SEQ ID NO: 14 and SEQ ID NO: 17.

Embodiment 18. The BAA of any one of embodiments 1 to 17, wherein the AB1 comprises an Fc region comprising an amino acid substitution in at least one of amino acid positions L234, L235, N297, and P331, as numbered by the EU index as set forth in Kabat, such that the BAA has reduced effector function.

Embodiment 19. The BAA of embodiment 18, wherein the AB1 comprises amino acid substitutions in at least two of amino acid positions L234, L235, and P331.

Embodiment 20. The BAA of any one of embodiments 18 to 19, wherein the AB1 comprises amino acid substitutions at amino acid positions L234, L235, and P331.

Embodiment 21. The BAA of any one of embodiments 18 to 20, wherein the AB1 comprises L234F, L235E, and P331S amino acid substitutions.

Embodiment 22. The BAA of any one of embodiments 1 to 21, wherein the AB1 comprises an Fc region comprising an amino acid substitution at N297.

Embodiment 23. The BAA of any one of embodiments 18 to 22, wherein the AB1 comprises L234F, L235E, P331S, and N297Q amino acid substitutions.

Embodiment 24. The BAA of any one of embodiments 18 to 23, wherein the heavy chain of AB1 comprises an amino acid sequence selected from the group consisting SEQ ID NO: 115, SEQ ID NO: 116, SEQ ID NO: 117, SEQ ID NO: 118, SEQ ID NO: 119, SEQ ID NO: 120, SEQ ID NO: 121 and SEQ ID NO: 122, as set forth in Table 4-2.

Embodiment 25. The BAA of any one of embodiments 18 to 23, wherein the heavy chain of AB1 comprises an amino acid sequence selected from the group consisting SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76, as set forth in Table 6.

Embodiment 26. The BAA of any one of embodiments 1 to 7, and 10 to 25, wherein the first target is selected from the group consisting of the targets presented in Table 9.

Embodiment 27. The BAA of any one of embodiments 1 to 26, wherein each of the first and second prodomains comprises a linker L1 and a linker L2 according to the formula (MM)-L1-(CM)-L2 in the N-terminal to C-terminal direction.

Embodiment 28. The BAA CI138, wherein the BAA comprises a heavy chain amino acid sequence as set forth in SEQ ID NO: 155 or SEQ ID NO:124 and a light chain amino acid sequence as set forth in SEQ ID NO: 132 or SEQ ID NO:140.

Embodiment 29. The BAA CI139, wherein the BAA comprises a heavy chain amino acid sequence as set forth in SEQ ID NO: 157 or SEQ ID NO:126 and a light chain amino acid sequence as set forth in SEQ ID NO: 132 or SEQ ID NO:140.

Embodiment 30. The BAA CI158, wherein the BAA comprises a heavy chain amino acid sequence as set forth in SEQ ID NO: 161 or SEQ ID NO:128 and a light chain amino acid sequence as set forth in SEQ ID NO: 132 or SEQ ID NO:140.

Embodiment 31. The BAA CI140, wherein the BAA comprises a heavy chain amino acid sequence as set forth in SEQ ID NO: 159 or SEQ ID NO:25 and a light chain amino acid sequence as set forth in SEQ ID NO: 132 or SEQ ID NO:140.

Embodiment 32. An activatable antibody (AA), wherein said AA, when activated, specifically binds to a target, and wherein said AA, when not activated, comprises the following structure:

a) at least one scFv comprising a light chain variable region (VL) linked to a heavy chain variable region (VH), wherein the VL is linked to the VH by a linker L3 comprising amino acid sequences selected from the group consisting of SEQ ID NO: 98 and SEQ ID NO: 108; and
b) a prodomain comprising:

i. a masking moiety (MM) coupled to the scFv, wherein the MM reduces or inhibits the binding of the scFV to its target when the AA is in an uncleaved state; and

ii. a cleavable moiety (CM) coupled to the scFv, wherein the CM is a polypeptide that functions as a substrate for a protease.

Embodiment 33. The AA of embodiment 32, wherein the target of the scFv is CD3_{ε}.

Embodiment 34. An activatable antibody (AA) wherein said AA, when activated, specifically binds to a target, and wherein said AA, when not activated, comprises the following structure:

a) an antibody or antigen binding fragment thereof (AB) that specifically binds to CD3_{ε}; and

b) a prodomain comprising:
i. a masking moiety (MM) coupled to the AB, wherein the MM reduces or inhibits the binding of the AB to the CD3_{ε} when the AA is in an uncleaved state, wherein the MM comprises the amino acid sequence SEQ ID NO: 105 or SEQ ID NO: 106; or SEQ ID NO: 107 and
ii. a cleavable moiety (CM) coupled to the AB, wherein the CM is a polypeptide that functions as a substrate for a protease.

Embodiment 35. The AA of any one of embodiments 32 to 34, wherein the CM comprises an amino acid sequence selected from the group consisting of SEQ ID NO:13-17.

Embodiment 36. The AA of any one of embodiments 32 to 34, wherein the CM comprises a substrate cleavable by a serine protease or an MMP.

Embodiment 37. The AA of any one of embodiments 32 to 34, wherein the CM comprises an amino acid sequence selected from the group consisting of SEQ ID NO: 18-56.

Embodiment 38. The AA of any one of embodiments 32 to 34, wherein the protease is an MMP.

Embodiment 39. The AA of any one of embodiments 32 to 34, wherein the protease is a serine protease.

Embodiment 40. The AA of any one of embodiments 32 to 39, wherein the VL comprises an amino acid sequence as set forth in SEQ ID NO: 1 or SEQ ID NO: 4 and the VH comprises an amino acid sequence as set forth in SEQ ID NO: 2 or SEQ ID NO: 3.

Embodiment 41. The AA of any one of embodiments 32 to 40, wherein the prodomain comprises a linker L1 and a linker L2 according to the formula (MM)-L1-(CM)-L2 in the N-terminal to C-terminal direction.

Embodiment 42. The AA or BAA of any one of embodiments 1 to 41, wherein the antigen binding fragment thereof is selected from the group consisting of a Fab fragment, a F(ab′)2 fragment, a scFv, a scAb, a dAb, a single domain heavy chain antibody, and a single domain light chain antibody.

Embodiment 43. The AA or BAA of any one of embodiments 1 to 42, wherein the antibody is a rodent antibody, a chimeric antibody, a humanized antibody, or a fully human monoclonal antibody.

Embodiment 44. The AA of any one of embodiments 1 to 43, wherein the AA is part of a BAA.

Embodiment 45. A pharmaceutical composition comprising the AA or BAA of any one of embodiments 1 to 44 and, optionally, a carrier.

Embodiment 46. The pharmaceutical composition of embodiment 45 comprising an additional agent.

Embodiment 47. The pharmaceutical composition of embodiment 46, wherein the additional agent is a therapeutic agent.

Embodiment 48. An isolated nucleic acid molecule encoding the AA or BAA of any one of embodiments 1 to 44.

Embodiment 49. A vector comprising an isolated nucleic acid molecule of embodiment 48.

Embodiment 50. A vector comprising the nucleic acid sequence of pEF1049.

Embodiment 51. A vector comprising the nucleic acid sequence of pEF1050.

Embodiment 52. A vector comprising the nucleic acid sequence of pEF1107.

Embodiment 53. A vector comprising the nucleic acid sequence of pEF1052.

Embodiment 54. A cell comprising any one of the vectors of any one of embodiments 50 to 53.

Embodiment 55. A cell comprising pEF1049 and pLW246.

Embodiment 56. A cell comprising pEF1050 and pLW246.

Embodiment 57. A cell comprising pEF1107 and pLW246

Embodiment 58. A cell comprising pEF1052 and pLW246.

Embodiment 59. A method of producing the AA or BAA of any one of embodiments 1 to 44 by culturing a cell under conditions that lead to expression of the AA or BAA, wherein the cell comprises a nucleic acid molecule of embodiment 48 or a vector of any one of embodiments 49 to 53, or a cell as set forth in any one of embodiments 54 to 58.

Embodiment 60. A method of treating, alleviating a symptom of, or delaying the progression of a disorder or disease comprising administering a therapeutically effective amount of the AA or BAA of any one of embodiments 1 to 44, or the pharmaceutical composition of any one of embodiments 45 to 47 to a subject in need thereof.

Embodiment 61. The method of embodiment 60, wherein the disorder or disease comprises disease cells expressing EGFR.

Embodiment 62. The method of any one of embodiments 60 or 61, wherein the disorder or disease is cancer.

Embodiment 63. The method of embodiment 62, wherein the cancer is bladder cancer, breast cancer, cervical cancer, cholangiocarcinoma, colorectal cancer, endometrial cancer, esophageal cancer, gastric cancer, glioblastoma, head and neck cancer, liver cancer, lung cancer, ovarian cancer, pancreatic cancer, prostate cancer, renal cancer, sarcoma, squamous cell cancer, or skin cancer.

Embodiment 64. A method of inhibiting angiogenesis in a subject comprising administering a therapeutically effective amount of the AA or BAA of any one of embodiments 1 to 44, or the pharmaceutical composition of any one of embodiments 45 to 47 to a subject in need thereof.

Embodiment 65. The method of any one of embodiments 60 to 64, wherein the method comprises administering an additional agent.

Embodiment 66. The method of embodiment 65 wherein the additional agent is a therapeutic agent.

Embodiment 67. A method of reducing damage to healthy tissue caused by an antibody binding to its target on healthy tissue as well as on diseased tissue, the method comprising administering to a subject in need thereof an AA, BAA or a pharmaceutical composition comprising an AA or BAA, wherein said AA or BAA is an AA or BAA of any one of the embodiments 1 to 44.

Embodiment 68. A method to improve tolerability of an antibody treatment comprising administering to a subject in need thereof an AA, BAA or a pharmaceutical composition comprising an AA or BAA, wherein said AA or BAA is an AA or BAA of any one of the embodiments 1 to 44.

Embodiment 69. A method to recruit T cells to tumor tissue comprising administering to a subject in need thereof an AA or BAA or a pharmaceutical composition comprising an AA or BAA, wherein said AA or BAA is an AA or BAA of any one of the embodiments 1 to 44.

Embodiment 70. An AA or BAA of any one of embodiments 1 to 44, or a pharmaceutical composition of any one of embodiments 45 to 47, for use in a method of treating, alleviating a symptom of, or delaying the progression of a disorder or disease.

Embodiment 71. The use of embodiment 70, wherein the disorder or disease comprises disease cells expressing EGFR.

Embodiment 72. The use of embodiment 70 or 71, wherein the disorder or disease is cancer.

Embodiment 73. The use of embodiment 72, wherein the cancer is bladder cancer, breast cancer, cervical cancer, cholangiocarcinoma, colorectal cancer, endometrial cancer, esophageal cancer, gastric cancer, glioblastoma, head and neck cancer, liver cancer, lung cancer, ovarian cancer, pancreatic cancer, prostate cancer, renal cancer, sarcoma, squamous cell cancer, or skin cancer.

Embodiment 74. An AA or BAA of any one of embodiments 1 to 44, or a pharmaceutical composition of any one of embodiments 45 to 47, for use in a method of inhibiting angiogenesis in a subject.

Embodiment 75. The use of any one of embodiments 70 to 74, wherein the method comprises the use of an additional agent.

Embodiment 76. The method of embodiment 75 wherein the additional agent is a therapeutic agent.

Embodiment 77. An AA or BAA of any one of embodiments 1 to 44, or a pharmaceutical composition of any one of embodiments 45 to 47, for use in a method of reducing damage to healthy tissue caused by an antibody binding to its target on healthy tissue as well as on diseased tissue.

Embodiment 78. An AA or BAA of any one of embodiments 1 to 44, or a pharmaceutical composition of any one of embodiments 45 to 47, for use in a method to improve tolerability of an antibody treatment.

Embodiment 79. An AA or BAA of any one of embodiments 1 to 44, or a pharmaceutical composition of any one of embodiments 45 to 47, for use in a method to recruit T cells to tumor tissue.

### EXAMPLES

The following examples are included for illustrative purposes and are not intend to limit the scope of the invention.

### Example 1. Sequences, Vector Construction and Expression of Antibodies, BAAs and Activated BAAs

### Antibodies of Interest

The molecules as provided in Table 11 below were constructed and tested. As indicated, activated molecules were produced as masked and proteolytically cleaved to produce the activated forms.

TABLE 11

| | | Heavy | Light |
|---|---|---|---|
| Molecule | | Chain | Chain |
| Name | Molecule Component Parts | Vector | Vector |
| CI106 | CF41-2008-C225v5Fcmt4- | pLW289 | pLW246 |
| | h20GG-0011-v16sc-H-N | | |
| CI138 | CF41-2008-C225v5Fcmt4- | pEF1049 | pLW246 |
| | h20GG-0011-v16(L3)sc-H-N | | |
| CI139 | CF41-2008-C225v5Fcmt4- | pEF1050 | pLW246 |
| | hCD1-0011-v16sc-H-N | | |
| CI158 | CF41-2008-C225v5Fcmt4- | pEF1107 | pLW246 |
| | hCD1 variant-0011-v16sc-H-N | | |
| CI140 | CF41-2008-C225v5Fcmt4- | pEF1052 | pLW246 |
| | hCD13-0011-v16sc-H-N | | |
| v12 | Anti-CD3 variant | HV12 | LV12 |
| v16 | Anti-CD3 variant | HV20 | LV12 |
| v19 | Anti-CD3 variant | HV20 | LV19 |
| v26 | Anti-CD3 variant | HV12 | LV19 |

The sequences of the molecules and vectors are provided below. Brackets denote some of the component parts of the molecules presented. In some sequences, linkers are provided. Underlined amino acids denote predicted CDR sequences.

CI106: CF41-2008-C225v5Fcmt4-h20GG-0011-v16sc-H-N
pLW289: HC h20GG-0011-v16sc-C225v5Fcmt4 (H-N)
Nucleotide Sequence
[spacer SEQ ID NO: 134][pLW289 without spacer SEQ ID NO: 135]
(SEQ ID NO: 129)
CAAGGCCAGTCTGGATCCGGTTATCTGTGGGGTTGCGAGTGGAATTGCGGAGGG
ATCACTACAGGCTCGAGCGGTGGCAGCGGTGGCTCTGGTGGTCTGAGCGGCCGTT
CCGATGATCATGGCGGCGGTTCTCAAACTGTAGTAACTCAAGAACCAAGCTTCTC
CGTCTCCCCTGGGGGAACAGTCACACTTACCTGCCGAAGTAGTACAGGTGCTGTT
ACGACCAGTAACTATGCCAATTGGGTACAACAAACGCCTGGTCAGGCTCCGCGC
GGATTGATAGGAGGCACGAATAAACGGGCACCCGGTGTCCCGGACAGATTCAGC
GGAAGCATACTCGGTAATAAGGCAGCTCTTACTATCACTGGGGCCCAAGCTGAT
GATGAAAGTGATTATTATTGTGCGCTCTGGTACAGCAACCTCTGGGTGTTTGGGG
GTGGCACGAAACTTACTGTCTTGGGCGGCGGCGGATCAGGGGGAGGTGGCTCTG
GAGGAGGAGGCTCAGAAGTCCAACTGGTCGAATCCGGGGGAGGGCTCGTACAGC
CGGGTGGGTCCCTCAAACTCTCTTGTGCGGCCTCAGGGTTTACCTTCAGTACATA
CGCGATGAATTGGGTCCGGCAGGCCAGTGGGAAAGGGCTCGAATGGGTAGGACG
AATCCGATCAAAATACAACAACTACGCTACTTATTACGCTGATTCCGTGAAGGAC
AGATTCACAATATCCCGCGACGATAGCAAGAATACGGCATATCTTCAGATGAATT
CTCTTAAAACTGAGGATACCGCTGTGTATTACTGCACAAGACATGGTAATTTTGG
AAACTCATATGTCTCTTGGTTCGCTTATTGGGGACAGGGCACGTTGGTTACCGTG
TCTAGCGGAGGTGGTGGATCCCAGGTGCAGCTGAAACAGAGCGGCCCGGGCCTG
GTGCAGCCGAGCCAGAGCCTGAGCATTACCTGCACCGTGAGCGGCTTTAGCCTG
ACCAACTATGGCGTGCATTGGGTGCGCCAGAGCCCGGGCAAAGGCCTGGAATGG
CTGGGCGTGATTTGGAGCGGCGGCAACACCGATTATAACACCCCGTTTACCAGCC
GCCTGAGCATTAACAAAGATAACAGCAAAAGCCAGGTGTTTTTTAAAATGAACA
GCCTGCAAAGCCAGGATACCGCGATTTATTATTGCGCGCGCGCGCTGACCTATTA
TGATTATGAATTTGCGTATTGGGGCCAGGGCACCCTGGTGACCGTGAGCGCGGCT
AGCACCAAGGGCCCATCGGTCTTCCCCCTGGCACCCTCCTCCAAGAGCACCTCTG
GGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACACC
AAGGTGGACAAGAAAGTTGAGCCCAAATCTTGTGACAAAACTCACACATGCCCA
CCGTGCCCAGCACCTGAATTTGAAGGGGGACCGTCAGTCTTCCTCTTCCCCCCAA
AACCCAAGGACACCCTCATGATCTCCCGGACCCCTGAGGTCACATGCGTGGTGGT
GGACGTGAGCCACGAAGACCCTGAGGTCAAGTTCAACTGGTACGTGGACGGCGT
GGAGGTGCATAATGCCAAGACAAAGCCGCGGGAGGAGCAGTACCAGAGCACGT
ACCGTGTGGTCAGCGTCCTCACCGTCCTGCACCAGGACTGGCTGAATGGCAAGG
AGTACAAGTGCAAGGTCTCCAACAAAGCCCTCCCAGCCTCAATCGAGAAAACCA
TCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCT
TCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCATGCTC
CGTGATGCATGAGGCTCTGCACAACCACTACACGCAGAAGAGCCTCTCCCTGTCT
CCGGGTAAA
Amino Acid Sequence
[spacer SEQ ID NO: 133][pLW289 without spacer SEQ ID NO: 136]
(SEQ ID NO: 130)
QGQSGS[GYLWGCEWNCGGITT][GSSGGSGGSGG][LSGRSDDH][GGGS]QTVVTQEP
SFSVSPGGTVTLTCRSSTGAVTTSNYANWVQQTPGQAPRGLIGGTNKRAPGVPDRFS
GSILGNKAALTITGAQADDESDYYCALWYSNLWVFGGGTKLTVL[GGGGSGGGGSG
GGGS]EVQLVESGGGLVQPGGSLKLSCAASGFTFSTYAMNWVRQASGKGLEWVGRI
RSKYNNYATYYADSVKDRFTISRDDSKNTAYLQMNSLKTEDTAVYYCTRHGNFGNS
YVSWFAYWGQGTLVTVSS[GGGGS ]QVQLKQSGPGLVQPSQSLSITCTVSGFSLTNY
GVHWVRQSPGKGLEWLGVIWSGGNTDYNTPFTSRLSINKDNSKSQVFFKMNSLQSQ
DTAIYYCARALTYYDYEFAYWGQGTLVTVSAASTKGPSVFPLAPSSKSTSGGTAALG
CLVKDYFPEPVTVSWNSGALTSGVHTFPAVLQSSGLYSLSSVVTVPSSSLGTQTYICN
VNHKPSNTKVDKKVEPKSCDKTHTCPPCPAPEFEGGPSVFLFPPKPKDTLMISRTPEV
TCVVVDVSHEDPEVKFNWYVDGVEVHNAKTKPREEQYQSTYRVVSVLTVLHQDW
LNGKEYKCKVSNKALPASIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSV
MHEALHNHYTQKSLSLSPGK
pLW246: LC CF41-2008-C225v5
Nucleotide Sequence
[spacer SEQ ID NO: 137][pLW246 without spacer SEQ ID NO: 139]
(SEQ ID NO: 131)
CAAGGCCAGTCTGGCCAAGGTCTTAGTTGTGAAGGTTGGGCGATGAATAGAGAA
CAATGTCGAGCCGGAGGTGGCTCGAGCGGCGGCTCTATCTCTTCCGGACTGCTGT
CCGGCAGATCCGACCAGCACGGCGGAGGATCCCAAATCCTGCTGACACAGTCTC
CTGTCATACTGAGTGTCTCCCCCGGCGAGAGAGTCTCTTTCTCATGTCGGGCCAG
TCAGTCTATTGGGACTAACATACACTGGTACCAGCAACGCACCAACGGAAGCCC
GCGCCTGCTGATTAAATATGCGAGCGAAAGCATTAGCGGCATTCCGAGCCGCTTT
AGCGGCAGCGGCAGCGGCACCGATTTTACCCTGAGCATTAACAGCGTGGAAAGC
GAAGATATTGCGGATTATTATTGCCAGCAGAACAACAACTGGCCGACCACCTTTG
GCGCGGGCACCAAACTGGAACTGAAACGTACGGTGGCTGCACCATCTGTCTTCAT
CTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAG
AGCTTCAACAGGGGAGAGTGT
Amino Acid Sequence
[spacer SEQ ID NO: 138][pLW246 without spacer SEQ ID NO: 140]
(SEQ ID NO: 132)
QGQSGQG[LSCEGWAMNREQCRA][GGGSSGGS][ISSGLLSGRSDQH][GGGS]QILLT
QSPVILSVSPGERVSFSCRASQSIGTNIHWYQQRTNGSPRLLIKYASESISGIPSRFSGSG
SGTDFTLSINSVESEDIADYYCQQNNNWPTTFGAGTKLELKRTVAAPSVFIFPPSDEQ
LKSGTASVVCLLNNFYPREAKVQWKVDNALQSGNSQESVTEQDSKDSTYSLSSTLTL
SKADYEKHKVYACEVTHQGLSSPVTKSFNRGEC
CI138: CF41-2008-C225v5Fcmt4-h20GG-0011-v16(L3)sc-H-N
(wherein L3 is SEQ ID NO: 108)
pEF1049: HC h20GG-0011-v16(L3)sc-C225v5Fcmt4 (H-N)
Nucleotide Sequence
[spacer SEQ ID NO: 141][pEF1049 without spacer SEQ ID NO: 123]
(SEQ ID NO: 154)
ATGGACATGAGGGTCCCCGCTCAGCTCCTGGGGCTCCTGCTACTATGGCTCCGCG
GTGCTAGATGTCAAGGCCAGTCTGGATCCGGTTATCTGTGGGGTTGCGAGTGGAA
TTGCGGAGGGATCACTACAGGCTCGAGCGGTGGCAGCGGTGGCTCTGGTGGTCT
GAGCGGCCGTTCCGATGATCATGGCGGCGGTTCTCAAACTGTAGTAACTCAAGA
ACCAAGCTTCTCCGTCTCCCCTGGGGGAACAGTCACACTTACCTGCCGAAGTAGT
ACAGGTGCTGTTACGACCAGTAACTATGCCAATTGGGTACAACAAACGCCTGGTC
AGGCTCCGCGCGGATTGATAGGAGGCACGAATAAACGGGCACCCGGTGTCCCGG
ACAGATTCAGCGGAAGCATACTCGGTAATAAGGCAGCTCTTACTATCACTGGGG
CCCAAGCTGATGATGAAAGTGATTATTATTGTGCGCTCTGGTACAGCAACCTCTG
GGTGTTTGGGGGTGGCACGAAACTTACTGTCTTGGGCTCCACCTCCGGCTCCGGC
AAGCCCGGCTCCTCCGAGGGCTCCACCGAAGTCCAACTGGTCGAATCCGGGGGA
GGGCTCGTACAGCCGGGTGGGTCCCTCAAACTCTCTTGTGCGGCCTCAGGGTTTA
CCTTCAGTACATACGCGATGAATTGGGTCCGGCAGGCCAGTGGGAAAGGGCTCG
AATGGGTAGGACGAATCCGATCAAAATACAACAACTACGCTACTTATTACGCTG
ATTCCGTGAAGGACAGATTCACAATATCCCGCGACGATAGCAAGAATACGGCAT
ATCTTCAGATGAATTCTCTTAAAACTGAGGATACCGCTGTGTATTACTGCACAAG
ACATGGTAATTTTGGAAACTCATATGTCTCTTGGTTCGCTTATTGGGGACAGGGC
ACGTTGGTTACCGTGTCTAGCGGAGGTGGTGGATCCCAGGTGCAGCTGAAACAG
AGCGGCCCGGGCCTGGTGCAGCCGAGCCAGAGCCTGAGCATTACCTGCACCGTG
AGCGGCTTTAGCCTGACCAACTATGGCGTGCATTGGGTGCGCCAGAGCCCGGGC
AAAGGCCTGGAATGGCTGGGCGTGATTTGGAGCGGCGGCAACACCGATTATAAC
ACCCCGTTTACCAGCCGCCTGAGCATTAACAAAGATAACAGCAAAAGCCAGGTG
TTTTTTAAAATGAACAGCCTGCAAAGCCAGGATACCGCGATTTATTATTGCGCGC
GCGCGCTGACCTATTATGATTATGAATTTGCGTATTGGGGCCAGGGCACCCTGGT
GACCGTGAGCGCGGCTAGCACCAAGGGCCCATCGGTCTTCCCCCTGGCACCCTCC
TCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTGAATCA
CAAGCCCAGCAACACCAAGGTGGACAAGAAAGTTGAGCCCAAATCTTGTGACAA
AACTCACACATGCCCACCGTGCCCAGCACCTGAATTTGAAGGGGGACCGTCAGT
CTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGA
GCAGTACCAGAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGCC
TCAATCGAGAAAACCATCTCCAAAGCCAAAGGGCAGCCCCGAGAACCACAGGTG
TACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGA
AGAGCCTCTCCCTGTCTCCGGGTAAA
Amino Acid Sequence
[spacer SEQ ID NO: 133][pEF1049 without spacer SEQ ID NO: 124]
(SEQ ID NO: 155)
QGQSGS[GYLWGCEWNCGGITT][GSSGGSGGSGG][LSGRSDDH][GGGS]QTVVTQEP
SFSVSPGGTVTLTCRSSTGAVTTSNYANWVQQTPGQAPRGLIGGTNKRAPGVPDRFS
GSILGNKAALTITGAQADDESDYYCALWYSNLWVFGGGTKLTVL[GSTSGSGKPGSS
EGST]EVQLVESGGGLVQPGGSLKLSCAASGFTFSTYAMNWVRQASGKGLEWVGRI
RSKYNNYATYYADSVKDRFTISRDDSKNTAYLQMNSLKTEDTAVYYCTRHGNFGNS
YVSWFAYWGQGTLVTVSS[GGGGS]QVQLKQSGPGLVQPSQSLSITCTVSGFSLTNY
GVHWVRQSPGKGLEWLGVIWSGGNTDYNTPFTSRLSINKDNSKSQVFFKMNSLQSQ
DTAIYYCARALTYYDYEFAYWGQGTLVTVSAASTKGPSVFPLAPSSKSTSGGTAALG
CLVKDYFPEPVTVSWNSGALTSGVHTFPAVLQSSGLYSLSSVVTVPSSSLGTQTYICN
VNHKPSNTKVDKKVEPKSCDKTHTCPPCPAPEFEGGPSVFLFPPKPKDTLMISRTPEV
TCVVVDVSHEDPEVKFNWYVDGVEVHNAKTKPREEQYQSTYRVVSVLTVLHQDW
LNGKEYKCKVSNKALPASIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSV
MHEALHNHYTQKSLSLSPGK
pLW246: LC CF41-2008-C225v5
Sequences provided above
CI139: CF41-2008-C225v5Fcmt4-hCD1-0011-v16sc-H-N
pEF1050: HC hCD1-0011-v16sc-C225v5Fcmt4 (H-N)
Nucleotide Sequence
[spacer SEQ ID NO: 141][pEF1050 without spacer SEQ ID NO: 125]
(SEQ ID NO: 156)
ATGGACATGAGGGTCCCCGCTCAGCTCCTGGGGCTCCTGCTACTATGGCTCCGCG
GTGCTAGATGTCAAGGCCAGTCTGGATCCATGATGTACTGCGGCGGAAACGAGA
TCTTCTGCGAGCCCAGAGGCGGCTCGAGCGGTGGCAGCGGTGGCTCTGGTGGTCT
GAGCGGCCGTTCCGATGATCATGGCGGCGGTTCTCAAACTGTAGTAACTCAAGA
ACCAAGCTTCTCCGTCTCCCCTGGGGGAACAGTCACACTTACCTGCCGAAGTAGT
ACAGGTGCTGTTACGACCAGTAACTATGCCAATTGGGTACAACAAACGCCTGGTC
AGGCTCCGCGCGGATTGATAGGAGGCACGAATAAACGGGCACCCGGTGTCCCGG
ACAGATTCAGCGGAAGCATACTCGGTAATAAGGCAGCTCTTACTATCACTGGGG
CCCAAGCTGATGATGAAAGTGATTATTATTGTGCGCTCTGGTACAGCAACCTCTG
GGTGTTTGGGGGTGGCACGAAACTTACTGTCTTGGGCGGCGGCGGATCAGGGGG
AGGTGGCTCTGGAGGAGGAGGCTCAGAAGTCCAACTGGTCGAATCCGGGGGAGG
GCTCGTACAGCCGGGTGGGTCCCTCAAACTCTCTTGTGCGGCCTCAGGGTTTACC
TTCAGTACATACGCGATGAATTGGGTCCGGCAGGCCAGTGGGAAAGGGCTCGAA
TGGGTAGGACGAATCCGATCAAAATACAACAACTACGCTACTTATTACGCTGATT
CCGTGAAGGACAGATTCACAATATCCCGCGACGATAGCAAGAATACGGCATATC
TTCAGATGAATTCTCTTAAAACTGAGGATACCGCTGTGTATTACTGCACAAGACA
TGGTAATTTTGGAAACTCATATGTCTCTTGGTTCGCTTATTGGGGACAGGGCACG
TTGGTTACCGTGTCTAGCGGAGGTGGTGGATCCCAGGTGCAGCTGAAACAGAGC
GGCCCGGGCCTGGTGCAGCCGAGCCAGAGCCTGAGCATTACCTGCACCGTGAGC
GGCTTTAGCCTGACCAACTATGGCGTGCATTGGGTGCGCCAGAGCCCGGGCAAA
GGCCTGGAATGGCTGGGCGTGATTTGGAGCGGCGGCAACACCGATTATAACACC
CCGTTTACCAGCCGCCTGAGCATTAACAAAGATAACAGCAAAAGCCAGGTGTTTT
TTAAAATGAACAGCCTGCAAAGCCAGGATACCGCGATTTATTATTGCGCGCGCGC
GCTGACCTATTATGATTATGAATTTGCGTATTGGGGCCAGGGCACCCTGGTGACC
GTGAGCGCGGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAG
CCCAGCAACACCAAGGTGGACAAGAAAGTTGAGCCCAAATCTTGTGACAAAACT
CACACATGCCCACCGTGCCCAGCACCTGAATTTGAAGGGGGACCGTCAGTCTTCC
TCTTCCCCCCAAAACCCAAGGACACCCTCATGATCTCCCGGACCCCTGAGGTCAC
ATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGT
ACCAGAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCTCAAT
CGAGAAAACCATCTCCAAAGCCAAAGGGCAGCCCCGAGAACCACAGGTGTACAC
CCTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTC
TTCCTCTACAGCAAGCTCACCGTGGACAAGAGCAGGTGGCAGCAGGGGAACGTC
TTCTCATGCTCCGTGATGCATGAGGCTCTGCACAACCACTACACGCAGAAGAGCC
TCTCCCTGTCTCCGGGTAAA
Amino Acid Sequence
[spacer SEQ ID NO: 133][pEF1050 without spacer SEQ ID NO: 126]
(SEQ ID NO: 157)
QGQSGS[MMYCGGNEIFCEPRG][GSSGGSGGSGG][LSGRSDDH][GGGS]QTVVTQEP
SFSVSPGGTVTLTCRSSTGAVTTSNYANWVQQTPGQAPRGLIGGTNKRAPGVPDRFS
GSILGNKAALTITGAQADDESDYYCALWYSNLWVFGGGTKLTVL[GGGGSGGGGSG
GGGS]EVQLVESGGGLVQPGGSLKLSCAASGFTFSTYAMNWVRQASGKGLEWVGRI
RSKYNNYATYYADSVKDRFTISRDDSKNTAYLQMNSLKTEDTAVYYCTRHGNFGNS
YVSWFAYWGQGTLVTVSS[GGGGS]QVQLKQSGPGLVQPSQSLSITCTVSGFSLTNY
GVHWVRQSPGKGLEWLGVIWSGGNTDYNTPFTSRLSINKDNSKSQVFFKMNSLQSQ
DTAIYYCARALTYYDYEFAYWGQGTLVTVSAASTKGPSVFPLAPSSKSTSGGTAALG
CLVKDYFPEPVTVSWNSGALTSGVHTFPAVLQSSGLYSLSSVVTVPSSSLGTQTYICN
VNHKPSNTKVDKKVEPKSCDKTHTCPPCPAPEFEGGPSVFLFPPKPKDTLMISRTPEV
TCVVVDVSHEDPEVKFNWYVDGVEVHNAKTKPREEQYQSTYRVVSVLTVLHQDW
LNGKEYKCKVSNKALPASIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSV
MHEALHNHYTQKSLSLSPGK
CI158: CF41-2008-C225v5Fcmt4-hCD1 variant-0011-v16sc-H-N
pEF1107: HC hCD1 variant-0011-v16sc-C225v5Fcmt4 (H-N)
Nucleotide Sequence
[spacer SEQ ID NO: 141][pEF1107 without spacer SEQ ID NO: 127]
(SEQ ID NO: 160)
ATGGACATGAGGGTCCCCGCTCAGCTCCTGGGGCTCCTGCTACTATGGCTCCGCG
GTGCTAGATGTCAAGGCCAGTCTGGATCCATGATGTACTGCGGCGGAAACGAGA
TCTTCTGCGGCCCTAGAGGCGGCTCGAGCGGTGGCAGCGGTGGCTCTGGTGGTCT
GAGCGGCCGTTCCGATGATCATGGCGGCGGTTCTCAAACTGTAGTAACTCAAGA
ACCAAGCTTCTCCGTCTCCCCTGGGGGAACAGTCACACTTACCTGCCGAAGTAGT
ACAGGTGCTGTTACGACCAGTAACTATGCCAATTGGGTACAACAAACGCCTGGTC
AGGCTCCGCGCGGATTGATAGGAGGCACGAATAAACGGGCACCCGGTGTCCCGG
ACAGATTCAGCGGAAGCATACTCGGTAATAAGGCAGCTCTTACTATCACTGGGG
CCCAAGCTGATGATGAAAGTGATTATTATTGTGCGCTCTGGTACAGCAACCTCTG
GGTGTTTGGGGGTGGCACGAAACTTACTGTCTTGGGCGGCGGCGGATCAGGGGG
AGGTGGCTCTGGAGGAGGAGGCTCAGAAGTCCAACTGGTCGAATCCGGGGGAGG
GCTCGTACAGCCGGGTGGGTCCCTCAAACTCTCTTGTGCGGCCTCAGGGTTTACC
TTCAGTACATACGCGATGAATTGGGTCCGGCAGGCCAGTGGGAAAGGGCTCGAA
TGGGTAGGACGAATCCGATCAAAATACAACAACTACGCTACTTATTACGCTGATT
CCGTGAAGGACAGATTCACAATATCCCGCGACGATAGCAAGAATACGGCATATC
TTCAGATGAATTCTCTTAAAACTGAGGATACCGCTGTGTATTACTGCACAAGACA
TGGTAATTTTGGAAACTCATATGTCTCTTGGTTCGCTTATTGGGGACAGGGCACG
TTGGTTACCGTGTCTAGCGGAGGTGGTGGATCCCAGGTGCAGCTGAAACAGAGC
GGCCCGGGCCTGGTGCAGCCGAGCCAGAGCCTGAGCATTACCTGCACCGTGAGC
GGCTTTAGCCTGACCAACTATGGCGTGCATTGGGTGCGCCAGAGCCCGGGCAAA
GGCCTGGAATGGCTGGGCGTGATTTGGAGCGGCGGCAACACCGATTATAACACC
CCGTTTACCAGCCGCCTGAGCATTAACAAAGATAACAGCAAAAGCCAGGTGTTTT
TTAAAATGAACAGCCTGCAAAGCCAGGATACCGCGATTTATTATTGCGCGCGCGC
GCTGACCTATTATGATTATGAATTTGCGTATTGGGGCCAGGGCACCCTGGTGACC
GTGAGCGCGGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAG
CCCAGCAACACCAAGGTGGACAAGAAAGTTGAGCCCAAATCTTGTGACAAAACT
CACACATGCCCACCGTGCCCAGCACCTGAATTTGAAGGGGGACCGTCAGTCTTCC
TCTTCCCCCCAAAACCCAAGGACACCCTCATGATCTCCCGGACCCCTGAGGTCAC
ATGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAGT
ACCAGAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCTCAAT
CGAGAAAACCATCTCCAAAGCCAAAGGGCAGCCCCGAGAACCACAGGTGTACAC
CCTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTC
TTCCTCTACAGCAAGCTCACCGTGGACAAGAGCAGGTGGCAGCAGGGGAACGTC
TTCTCATGCTCCGTGATGCATGAGGCTCTGCACAACCACTACACGCAGAAGAGCC
TCTCCCTGTCTCCGGGTAAA
Amino Acid Sequence
[spacer SEQ ID NO: 133][pEF1107 without spacer SEQ ID NO: 128]
(SEQ ID NO: 161)
QGQSGS[MMYCGGNEIFCGPRG][GSSGGSGGSGG][LSGRSDDH][GGGS]QTVVTQEP
SFSVSPGGTVTLTCRSSTGAVTTSNYANWVQQTPGQAPRGLIGGTNKRAPGVPDRFS
GSILGNKAALTITGAQADDESDYYCALWYSNLWVFGGGTKLTVL[GGGGSGGGGSG
GGGS]EVQLVESGGGLVQPGGSLKLSCAASGFTFSTYAMNWVRQASGKGLEWVGRI
RSKYNNYATYYADSVKDRFTISRDDSKNTAYLQMNSLKTEDTAVYYCTRHGNFGNS
YVSWFAYWGQGTLVTVSS[GGGGS]QVQLKQSGPGLVQPSQSLSITCTVSGFSLTNY
GVHWVRQSPGKGLEWLGVIWSGGNTDYNTPFTSRLSINKDNSKSQVFFKMNSLQSQ
DTAIYYCARALTYYDYEFAYWGQGTLVTVSAASTKGPSVFPLAPSSKSTSGGTAALG
CLVKDYFPEPVTVSWNSGALTSGVHTFPAVLQSSGLYSLSSVVTVPSSSLGTQTYICN
VNHKPSNTKVDKKVEPKSCDKTHTCPPCPAPEFEGGPSVFLFPPKPKDTLMISRTPEV
TCVVVDVSHEDPEVKFNWYVDGVEVHNAKTKPREEQYQSTYRVVSVLTVLHQDW
LNGKEYKCKVSNKALPASIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSV
MHEALHNHYTQKSLSLSPGK
pLW246: LC CF41-2008-C225v5
Sequences provided above
CI140: CF41-2008-C225v5Fcmt4-hCD13-0011-v16sc-H-N
pEF1052: HC hCD13-0011-v16sc-C225v5Fcmt4 (H-N)
Nucleotide Sequence
[spacer SEQ ID NO: 141][pEF1052 without spacer SEQ ID NO: 142]
(SEQ ID NO: 158)
ATGGACATGAGGGTCCCCGCTCAGCTCCTGGGGCTCCTGCTACTATGGCTCCGCG
GTGCTAGATGTCAAGGCCAGTCTGGATCCGGTTATCTGTGGGGTTGCGAGTGGAA
TTGCGGAGGGTCCTCCCCCGGCTCGAGCGGTGGCAGCGGTGGCTCTGGTGGTCTG
AGCGGCCGTTCCGATGATCATGGCGGCGGTTCTCAAACTGTAGTAACTCAAGAAC
CAAGCTTCTCCGTCTCCCCTGGGGGAACAGTCACACTTACCTGCCGAAGTAGTAC
AGGTGCTGTTACGACCAGTAACTATGCCAATTGGGTACAACAAACGCCTGGTCA
GGCTCCGCGCGGATTGATAGGAGGCACGAATAAACGGGCACCCGGTGTCCCGGA
CAGATTCAGCGGAAGCATACTCGGTAATAAGGCAGCTCTTACTATCACTGGGGCC
CAAGCTGATGATGAAAGTGATTATTATTGTGCGCTCTGGTACAGCAACCTCTGGG
TGTTTGGGGGTGGCACGAAACTTACTGTCTTGGGCGGCGGCGGATCAGGGGGAG
GTGGCTCTGGAGGAGGAGGCTCAGAAGTCCAACTGGTCGAATCCGGGGGAGGGC
TCGTACAGCCGGGTGGGTCCCTCAAACTCTCTTGTGCGGCCTCAGGGTTTACCTT
CAGTACATACGCGATGAATTGGGTCCGGCAGGCCAGTGGGAAAGGGCTCGAATG
GGTAGGACGAATCCGATCAAAATACAACAACTACGCTACTTATTACGCTGATTCC
GTGAAGGACAGATTCACAATATCCCGCGACGATAGCAAGAATACGGCATATCTT
CAGATGAATTCTCTTAAAACTGAGGATACCGCTGTGTATTACTGCACAAGACATG
GTAATTTTGGAAACTCATATGTCTCTTGGTTCGCTTATTGGGGACAGGGCACGTT
GGTTACCGTGTCTAGCGGAGGTGGTGGATCCCAGGTGCAGCTGAAACAGAGCGG
CCCGGGCCTGGTGCAGCCGAGCCAGAGCCTGAGCATTACCTGCACCGTGAGCGG
CTTTAGCCTGACCAACTATGGCGTGCATTGGGTGCGCCAGAGCCCGGGCAAAGG
CCTGGAATGGCTGGGCGTGATTTGGAGCGGCGGCAACACCGATTATAACACCCC
GTTTACCAGCCGCCTGAGCATTAACAAAGATAACAGCAAAAGCCAGGTGTTTTTT
AAAATGAACAGCCTGCAAAGCCAGGATACCGCGATTTATTATTGCGCGCGCGCG
CTGACCTATTATGATTATGAATTTGCGTATTGGGGCCAGGGCACCCTGGTGACCG
TGAGCGCGGCTAGCACCAAGGGCCCATCGGTCTTCCCCCTGGCACCCTCCTCCAA
GAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAGCC
CAGCAACACCAAGGTGGACAAGAAAGTTGAGCCCAAATCTTGTGACAAAACTCA
CACATGCCCACCGTGCCCAGCACCTGAATTTGAAGGGGGACCGTCAGTCTTCCTC
TTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAGTAC
CAGAGCACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCTCAATCG
AGAAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTGG
TCAAAGGCTTCTATCCCAGCGACATCGCCGTGGAGTGGGAGAGCAATGGGCAGC
CGGAGAACAACTACAAGACCACGCCTCCCGTGCTGGACTCCGACGGCTCCTTCTT
CCTCTACAGCAAGCTCACCGTGGACAAGAGCAGGTGGCAGCAGGGGAACGTCTT
CTCATGCTCCGTGATGCATGAGGCTCTGCACAACCACTACACGCAGAAGAGCCTC
TCCCTGTCTCCGGGTAAA
Amino Acid Sequence
[spacer SEQ ID NO: 133][pEF1052 without spacer SEQ ID NO: 25]
(SEQ ID NO: 159)
QGQSGS[GYGWGCEWNCGGSSP][GSSGGSGGSGG][LSGRSDDH][GGGS]QTVVTQE
PSFSVSPGGTVTLTCRSSTGAVTTSNYANWVQQTPGQAPRGLIGGTNKRAPGVPDRF
SGSILGNKAALTITGAQADDESDYYCALWYSNLWVFGGGTKLTVL[GGGGSGGGGS
GGGGS]EVQLVESGGGLVQPGGSLKLSCAASGFTFSTYAMNWVRQASGKGLEWVG
RIRSKYNNYATYYADSVKDRFTISRDDSKNTAYLQMNSLKTEDTAVYYCTRHGNFG
NSYVSWFAYWGQGTLVTVSS[GGGGS]QVQLKQSGPGLVQPSQSLSITCTVSGFSLTN
YGVHWVRQSPGKGLEWLGVIWSGGNTDYNTPFTSRLSINKDNSKSQVFFKMNSLQS
QDTAIYYCARALTYYDYEFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEFEGGPSVFLFPPKPKDTLMISRTPE
VTCVVVDVSHEDPEVKFNWYVDGVEVHNAKTKPREEQYQSTYRVVSVLTVLHQD
WLNGKEYKCKVSNKALPASIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCS
VMHEALHNHYTQKSLSLSPGK
pLW246: LC CF41-2008-C225v5
Sequences provided above (SEQ ID NO: 131 and SEQ ID NO: 132)
Anti-CD3 scFv variant v12
(SEQ ID NO: 1)
Light chain LV12
(SEQ ID NO: 2)
Heavy chain HV12
Wherein linker L3 links the light chain and heavy chain, wherein
L3 is selected from SEQ ID NO: 98 and SEQ ID NO: 108.
Anti-CD3 scFv variant v16
(SEQ ID NO: 1)
Light chain LV12
(SEQ ID NO: 3)
Heavy chain HV20
Wherein linker L3 links the light chain and heavy chain, wherein
L3 is selected from SEQ ID NO: 98 and SEQ ID NO: 108.
Anti-CD3 scFv variant v19
(SEQ ID NO: 4)
Light chain LV19
(SEQ ID NO: 3)
Heavy chain HV20
Wherein linker L3 links the light chain and heavy chain, wherein
L3 is selected from SEQ ID NO: 98 and SEQ ID NO: 108.
Anti-CD3 scFv variant v26
(SEQ ID NO: 4)
Light chain LV19
(SEQ ID NO: 2)
Heavy chain HV12
Wherein linker L3 links the light chain and heavy chain, wherein
L3 is selected from SEQ ID NO: 98 and SEQ ID NO: 108.

### Vector Construction

The heavy and light chains were cloned separately into a mammalian expression vector using standard molecular biology techniques. Briefly, DNA fragments encoding the region of interest were amplified with primers binding to the terminal ends. Overlapping fragments were combined and amplified with flanking primers as needed to build the entire desired region. DNA fragments were subsequently cloned into the expression vector using a commercially available homologous recombination kit (MCLabs, South San Francisco, Calif.). The mammalian expression vector is a modified version of cDNA™3.1(+) from Invitrogen with selection marker of G418 or hygromycin. Mutations were introduced using the QuikChange Kit (Agilent, Santa Clara, Calif.).

### Expression of AAs and Dually Masked BAAs (BAAs)

AAs and BAAs were expressed in mammalian cells using a standard transfection kit (Life Technologies, Grand Island, N.Y.). Briefly, 293 cells were transfected with nucleic acids using a lipid-based system, following the manufacturer's recommended protocol. AAs and dually masked BAAs were purified from cell-free supernatant using Protein A beads (GE, Piscataway, N.J.) and concentrated using standard buffer exchange columns (Millipore, Temecula, Calif.).

### Example 2. Expression, Purification, and Dynamic Aggregation Tests of Dually Masked Bispecific Activatable Antibodies

Dually masked bispecific activatable antibodies were expressed using transient transfection in Expi293™ cells (Thermo Fisher Scientific, Waltham, Mass., Catalog A14635). Synthetic DNA sequences encoding the proteins and signal peptides were ordered from Integrated DNA Technologies and cloned into transient expression vectors containing the CMV promoter. Endotoxin-free plasmid DNA preparations were confirmed by DNA sequencing prior to use. Plasmid DNA was transiently transfected into Expi293™ cells using the manufacturer's recommended protocol. Supernatants were harvested four days post-transfection and purified using Protein A chromatography. The monomeric population for each expressed protein was purified by size exclusion chromatography (SEC) using a preparative-scale Superdex 200 10/30 GL column (GE Healthcare Life Sciences, Catalog #17517501) in 1×PBS pH 7.2 running buffer.

Purified proteins were analyzed by reducing SDS-PAGE. Protein aliquots (5 μg) were denatured for 10 min at 75° C. in sample buffer with reducing agent and separated on a 4-12% NuPAGE™ Bis-Tris gel (Thermo Fisher Scientific, Waltham, Mass., Catalog # NP0321) in MPOS buffer for 1 h at 15V and visualized after staining with InstantBlue™ for 1 h followed by destaining in water for at least 4 h. All proteins were confirmed to contain two polypeptides of the expected molecular weights (˜75 KDa and ˜25 KDa).

Monomer content of purified proteins was analyzed using analytical SEC. Protein aliquots (25 μg) were injected onto Superdex 200 Increase 5/150 GL column (GE Healthcare Life Sciences, Catalog #28906561) that was run with 1×PBS pH7.2 at 0.45 ml/min. All proteins were confirmed to contain >95% monomer content.

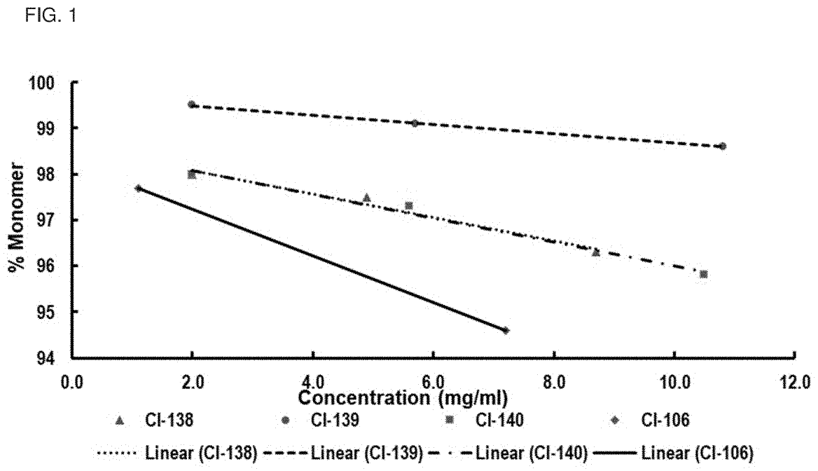

Concentration-dependent aggregation of the dually masked bispecific activatable antibodies was assessed by studying the relation between monomer content and concentration. Purified protein aliquots (>95% monomer content) were concentrated in a step-wise fashion using an Amicon Ultra 0.5 ml centrifugal concentrator (Millipore Sigma, Burlington, Mass.) at 14,000 rpm for 2 mins per step. The percent monomer at each concentration was determined by analytical SEC method described above. As shown in FIG. 1, CI106 showed a steeper drop in percent monomer with concentration compared to CI138, CI139, or CI140. CI139 showed the least tendency for concentration-dependent aggregation, whereas CI138 and CI140 showed a similar tendency for concentration-dependent aggregation between that of CI106 and CI139. The concentration-dependent aggregation tendency of these proteins was followed further by storing the concentrated samples (in PBS) at 4° C. for an additional 2-to-4 days. SEC results in Table 12 suggest that percent monomer continued to drop for CI106 (3% over 2 days), CI138 (4.5% over 4 days), and CI140 (4.3% over 4 days) but seemed to be stable for CI139 (0.3% over 4 days).

Each variant was SEC-purified, concentrated with a centrifugal concentrator and analyzed by SEC-HPLC.

TABLE 12

| CI-138 | | CI-139 | | CI-140 | | CI-106 | |
|---|---|---|---|---|---|---|---|
| conc. | % | conc. | % | conc. | % | conc. | % |
| mg/ | mono- | mg/ | mono- | mg/ | mono- | mg/ | mono- |
| ml | mer | ml | mer | ml | mer | ml | mer |
| 2.0 | 98 | 2 | 99.5 | 2 | 98 | 1.1 | 97.7 |
| 4.9 | 97.5 | 5.7 | 99.1 | 5.6 | 97.3 | 7.2 | 94.6 |
| 8.7 | 96.3 | 10.8 | 98.6 | 10.5 | 95.8 | | |

Storage at 4 C in PBS, pH 7.2 at highest concentration listed above.

| CI-138 @ 8.7 mg/ml | | CI-139 @ 10.8 mg/ml | | CI-140 @ 10.5 mg/ml | | CI-106 @ 7.2 mg/ml | |
|---|---|---|---|---|---|---|---|
| | % | | % | | % | | % |
| Duration | monomer | Duration | monomer | Duration | monomer | Duration | monomer |
| overnight | 95.2 | overnight | 98.1 | overnight | 94.8 | overnight | 92.2 |
| 2 days | ND | 2 days | ND | 2 days | ND | 2 days | 89.5 |
| 4 days | 90.7 | 4 days | 97.8 | 4 days | 90.5 | 4 days | ND |

### Example 3. CD3 and EGFR ELISA Assays for Dually Masked Bispecific Activatable Antibodies

ELISA assays were used to confirm and evaluate the binding of the intact dually masked bispecific activatable antibodies for CD3ε and EGFR ligands. The purified dually masked bispecific activatable antibodies were activated by incubation with recombinant human u-plasminogen activator/urokinase (R&D systems, cat #1310-SE) for 24 h in PBS buffer with 6% sucrose at 37 C, re-purified by protein A chromatography, and re-analyzed by SDS-PAGE and SEC.

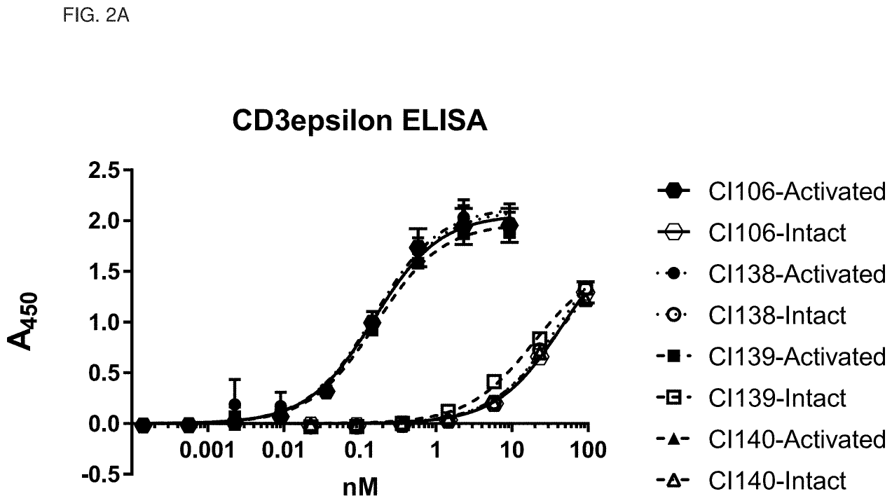

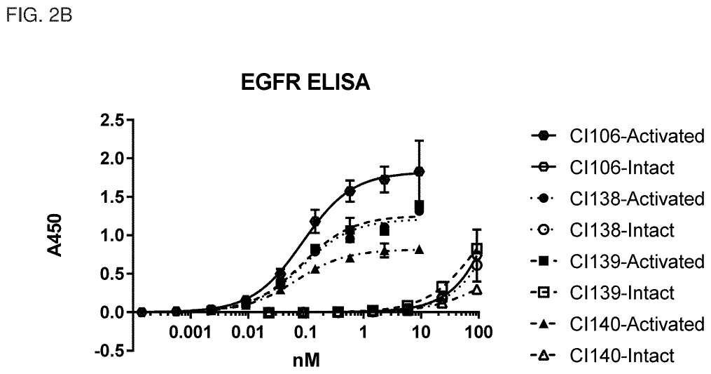

CD3_{ε} ELISA: Human CD3-epsilon protein (Acro Biosystems, Newark, Del., Catalog CDE-H5223) at 2 μg/ml in 1×PBS pH 7.2 (Thermo Fisher Scientific, Waltham, Mass. Catalog 20012043) was coated overnight at 4° C. on Nunc MaxiSorp™ 96 well plates (Thermo Fisher Scientific, Waltham, Mass. Catalog 439454). Plates were washed with 1×PBS pH 7.2, 0.05% Tween-20 using a plate washer, blocked with 200 μL/well of 1% BSA, 0.05% Tween 20 in 1×PBS pH 7.2 for 1 h at room temperature. The blocking solution was removed and serial dilutions of samples prepared in block buffer were added to the blocked plates for 1 h at room temperature. Plates were washed with plate washer prior to the addition of secondary antibody (1:25 k dilution Peroxidase AffiniPure Goat Anti-Human IgG (H+L specific), Jackson ImmunoResearch Catalog 109-035-088 in block buffer). Signal was developed using 1-Step™ TMB-ELISA Substrate solution (Thermo Fisher Scientific, Waltham, Mass. Catalog 34028), the reaction stopped with HCl, and the absorbance read at 450 nm. FIG. 2A shows that CI138, CI139, and CI140 behave similar to CI106 in both intact as well as activated forms.

EGFR ELISA: Human EGFR-Fc protein (R&D Biosciences 344-ER) at 1 μg/ml in 1×PBS pH 7.2 (Thermo Fisher Scientific, Waltham, Mass. Catalog 20012043) was coated overnight at 4° C. on Greiner Bio-One™ 96 well plates (Greiner, Monroe, N.C., Catalog 655061). Plates were washed with 1×PBS pH 7.2, 0.05% Tween-20 using a plate washer, then blocked with 200 μL/well of 1% BSA, 0.05% Tween 20 in 1×PBS pH 7.2 for 1 h at room temperature. Samples prepared in block buffer were added to the blocked plates for 1 h at room temperature. Plates were washed with plate washer prior to the addition of secondary antibody. Secondary antibody was added at 1:5000 dilution Peroxidase AffiniPure Mouse Anti-Human IgG, F(ab′)₂ fragment specific, in block buffer (Jackson ImmunoResearch, West Grove, Pa. Catalog 209-035-097). Signal was developed using 1-Step™ TMB-ELISA Substrate solution (Thermo Fisher Scientific, Waltham, Mass., Catalog 34028), the reaction stopped with HCl, and the absorbance read at 450 nm. FIG. 2B shows that intact CI138, CI139, and CI140 have similar EGFR binding as CI106. After activation, the EC50 for CI138, CI139, and CI140 looks similar to that of CI106 but the maximal signal is lower.

### Example 4. Biological Activity of Dually Masked Bispecific Activatable Antibodies

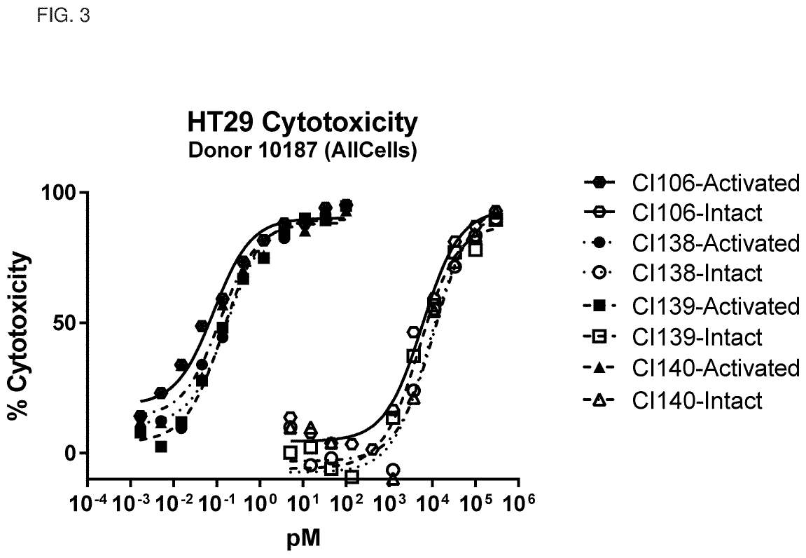

Biological activity of intact and activated bispecific activatable antibodies were assayed using cytotoxicity assays. Human PBMCs were purchased from AllCells (Alameda, Calif.) and co-cultured with EGFR expressing HT29-luc2 cells (Perkin Elmer, Inc., Waltham, Mass. (formally Caliper Life Sciences, Inc.) at a ratio of 10:1 in RPMI-1640+glutamax supplemented with 5% heat inactivated human serum (Sigma, Catalog H3667). Titrations of intact and activated CI106, CI138, CI139, and CI140 bispecific activatable antibodies were tested. After 48 hours, cytotoxicity was evaluated using the ONE-Glo™ Luciferase Assay System (Promega, Madison, Wis. Catalog E6130). Luminescence was measured on the Infinite® M200 Pro (Tecan Trading AG, Switzerland). Percent cytotoxicity was calculated and plotted in GraphPad PRISM with curve fit analysis. As shown in FIG. 3, CI138, CI139, and CI140 are similar to CI106 in intact as well as activated forms.

### Example 5. Dually Masked, Bispecific, Activatable Antibodies of the Embodiments Induced Regression of Established HT29-Luc2 Tumors in Mice

In this example, bispecific activatable antibodies CI106 and CI139 targeting EGFR and CD3ε were analyzed for the ability to induce regression or reduce growth of established HT-29-Luc2 xenograft tumors in human T-cell engrafted NSG mice.

The human colon cancer cell line HT29-luc2 was obtained from Perkin Elmer, Inc., Waltham, Mass. (formerly Caliper Life Sciences, Inc.) and cultured according to established procedures. Purified, frozen human PBMCs were obtained from Hemacare, Inc., Van Nuys, Calif. NSG™ (NOD.Cg-Prkdcscid Il2rg^{tm1Wj1}/SzJ) mice were obtained from The Jackson Laboratories, Bar Harbor, Me.

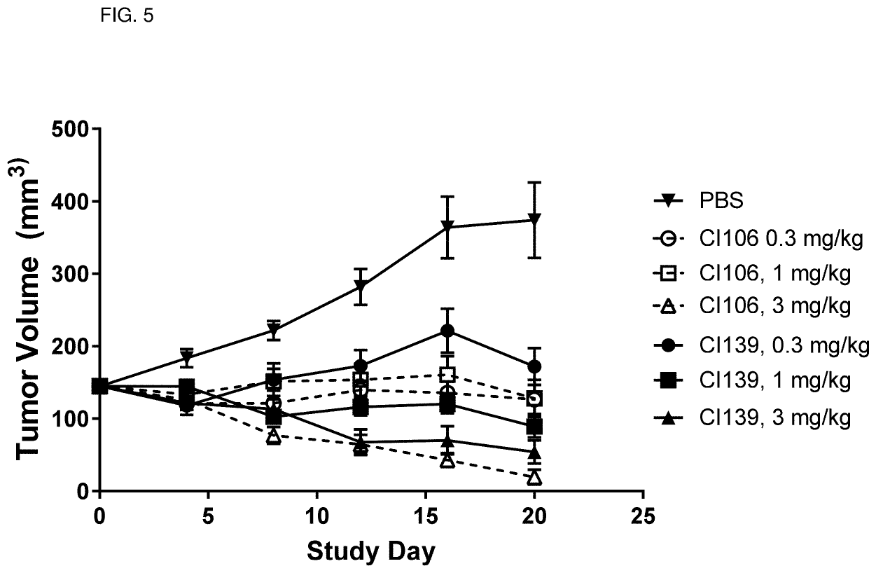

On day 0, each mouse was inoculated subcutaneously at the right flank with 2×10⁶ HT29-luc2 cells in 100 μL RPMI+Glutamax, serum-free medium. Previously frozen PBMCs from a single donor were administered (i.p.) on day 3 at a CD3+ T cell to tumor cell ratio of 1:1. When tumor volumes reached 150 mm³ (approximately day 12), mice were randomized, assigned to treatment groups and dosed i.v. according to Table 13. Tumor volume was measured twice weekly. As shown in FIG. 5, CI106 and CI139 have equivalent efficacy in this model.

TABLE 13
Groups and doses for HT2-9luc2 xenograft study.

| Group | Count | Treatment | Dose (mg/kg) |
|---|---|---|---|
| 1 | 8 | PBS | N/A |
| 2 | 8 | CI106 | 0.3 |
| 3 | 8 | CI106 | 1.0 |
| 4 | 8 | CI106 | 3.0 |
| 5 | 8 | CI139 | 0.3 |
| 6 | 8 | CI139 | 1.0 |
| 7 | 8 | CI139 | 3.0 |

### Example 6. Binding of Dually Masked, Bispecific, AAs to EGFR+HT-29 Cells and CD3ε+Jurkat Cells

To determine if the described EGFR and CD3ε masking peptides could inhibit binding in the context of a dually masked, bispecific, AA, a flow cytometry-based binding assay was performed.

HT-29-luc2 (Caliper) and Jurkat (Clone E6-1, ATCC, TIB-152) cells were cultured in RPMI-1640+glutamax (Life Technologies, Catalog 72400-047), 10% Heat Inactivated-Fetal Bovine Serum (HI-FBS, Life Technologies, Catalog 10438-026). The following bispecific, activated antibody CI106 (act-TCB), and dually masked, bispecific, AAs CI106 and CI139 were tested. Two versions of the CD3 mask were utilized, namely the CD3 mask in CI106 versus the CD3 mask in CI139.

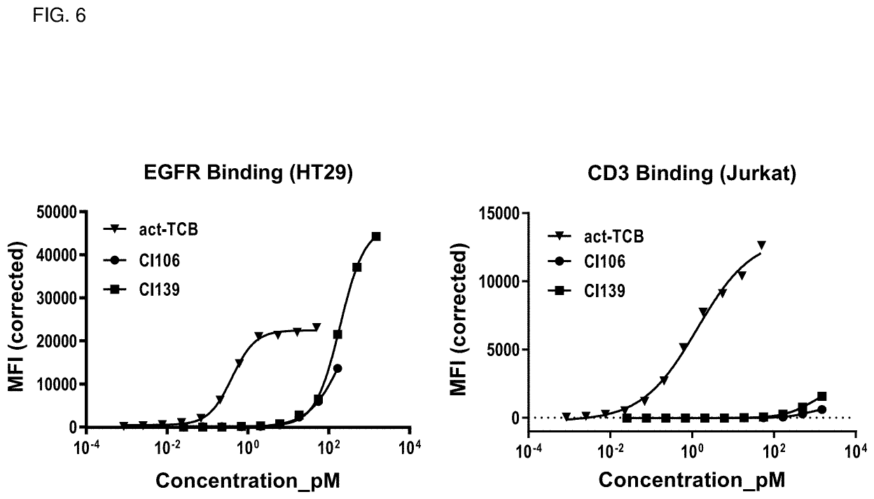

HT29-luc2 cells were detached with Versene™ (Life Technologies, Catalog 15040-066), washed, plated in 96 well plates at 150,000 cells/well, and re-suspended in 50 μL of primary antibody. Jurkat cells were counted and plated as described for HT29-luc2 cells. Titrations of primary antibody started at the concentrations indicated in FIG. 6 followed by 3-fold serial dilutions in FACS Stain Buffer+2% FBS (BD Pharmingen, Catalog 554656). Cells were incubated at 4° C. with shaking for about 1 hour, harvested, and washed with 2×200 μL of FACS Stain Buffer. Cells were resuspended in 50 μL of Alexa Fluor 488 conjugated anti-Human IgG Fc (10 μg/ml, Jackson ImmunoResearch) and incubated at 4° C. with shaking for about 1 hour. Cells were harvested, washed, and resuspended in a final volume of 200 μL of FACS Stain Buffer containing 2.5 μg/m17-AAD (BD Biosciences, Catalog 559925). Cells stained with secondary antibody alone were used as a negative control. Data was acquired on an Attune N×T Flow Cytometer and the median fluorescence intensity (MFI) of viable cells was calculated using FlowJo® V10 (Treestar). Background subtracted MFI data was graphed in GraphPad Prism using curve fit analysis.

FIG. 6 depicts reduced binding to both EGFR and CD3 of intact activatable bispecific antibodies C1106 and CI139 relative to the activated bispecific antibody as represented by a right shift of the binding curves.

### Example 7. Biological Activity of Dually Masked Bispecific Activatable Antibodies

Biological activity of intact and activated bispecific activatable antibodies was assayed using cytotoxicity assays. Human PBMCs were purchased from Stemcell Technologies (Vancouver, Canada) and co-cultured with EGFR expressing cancer cell lines HT29-luc2 (Perkin Elmer, Inc., Waltham, Mass. (formally Caliper Life Sciences, Inc.), NCI-N87 (ATCC), 0E33 (ATCC), SKBR3 (ATCC), or SKOV3 (ATCC) at a ratio of 10:1, 6:1, 8:1, 6:1, or 6:1 respectively in RPMI-1640+glutamax supplemented with 5% heat inactivated human serum (Sigma, Catalog H3667). Titrations of activated CI106 (act-TCB) and intact CI106 and CI139 bispecific activatable antibodies were tested. After 48 hours, cytotoxicity was evaluated using the ONE-Glo™ Luciferase Assay System (Promega, Madison, Wis. Catalog E6130) Luminescence was measured on the Infinite® M200 Pro (Tecan Trading AG, Switzerland). Percent cytotoxicity was calculated and plotted in GraphPad PRISM with curve fit analysis.

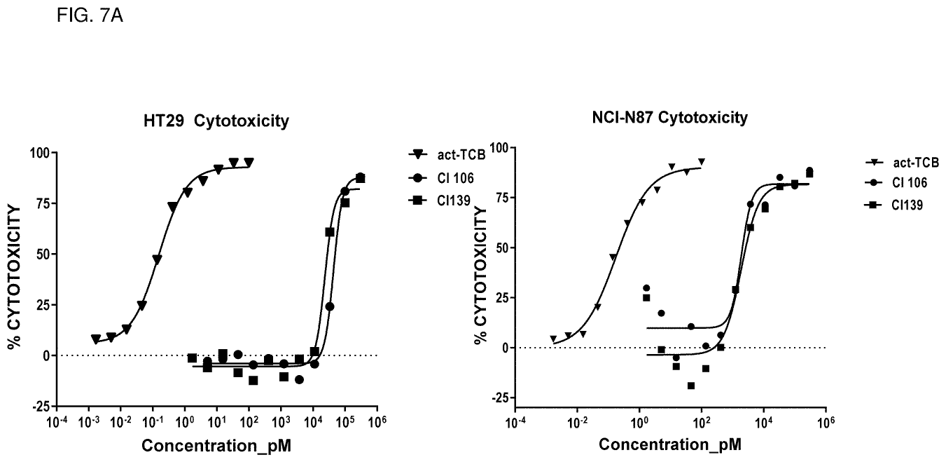

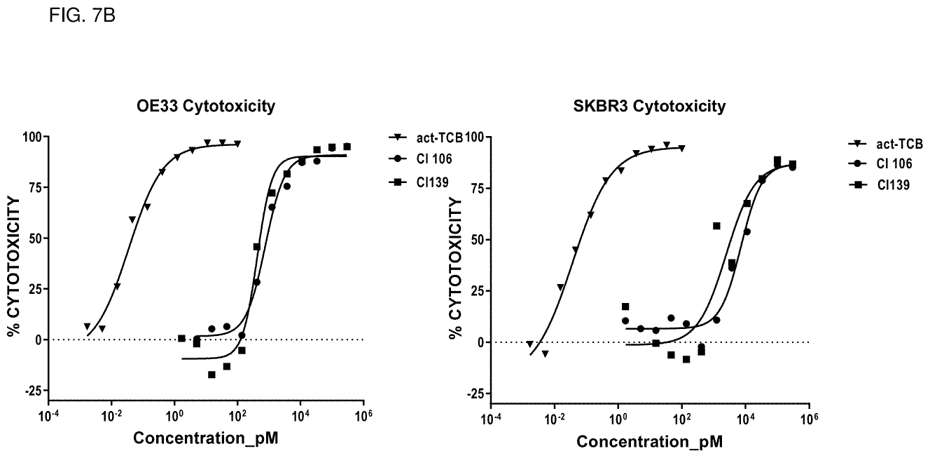

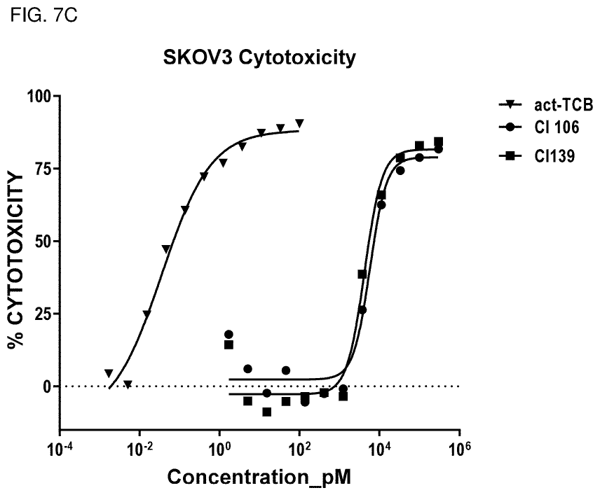

As shown in FIG. 7A-C and Table 14, the intact bispecific activatable antibodies have a shifted dose response curve relative to the activated bispecific antibody, and CI139 activity is similar to CI106 in these assays.

TABLE 14
in vitro masking efficiencies of bispecific activatable
antibodies on EGFR expressing cell lines

| | act-TCB | Masking | EGFR |
|---|---|---|---|
| Cell line | EC₅₀ (pM) | Efficiency | Receptor # |
| NCI-N87 | 0.17 | ~10,000x | 51,000 |
| HT29 | 0.21 | ~100,000x | 75,000 |
| SKOV3 | 0.04 | ~100,000x | 81,800 |
| OE33 | 0.04 | ~20,000x | 89,000 |
| SKBR3 | 0.04 | ~100,000x | No data |

### Example 8. Dually Masked, Bispecific, Activatable Antibodies of the Embodiments Induced Regression of Established HCT116 Tumors in Mice

In this example, bispecific activatable antibodies CI106 and CI139 targeting EGFR and CD3ε were analyzed for the ability to induce regression or reduce growth of established HCT116 xenograft tumors in human T-cell engrafted NSG mice.

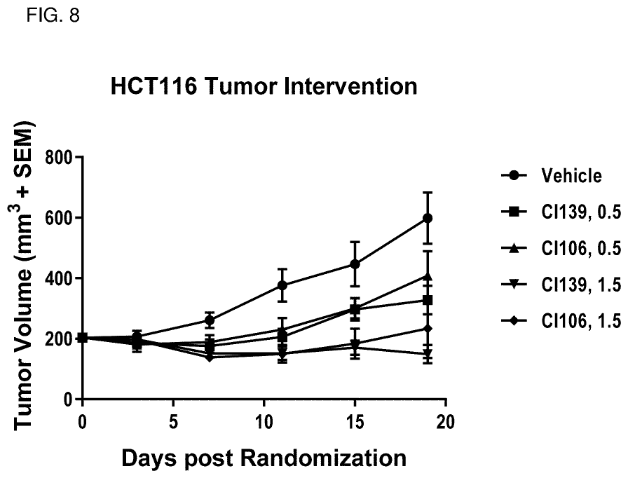

The human colon cancer cell line HCT116 was obtained from ATCC and was cultured in RPMI+Glutamax+10% PBS according to established procedures. The tumor model was carried out as described in Example 5. As shown in FIG. 8, CI106 and CI139 have equivalent efficacy in this model.

### Example 9. Pharmacokinetics of Dually Masked BAAs in Cynomolgus Monkeys

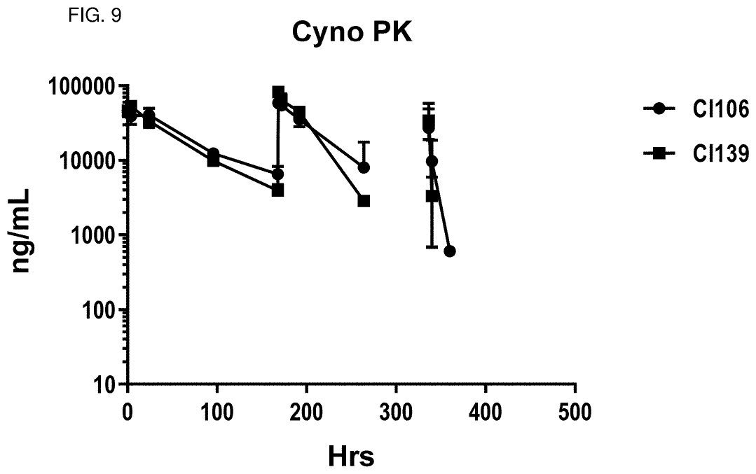

In this example dually masked, bispecific antibodies CI106 and CI139 were dosed at 3 mg/kg on day 1, 6 mg/kg on day 8, and 6 mg/kg on day 15 in cynomolgus monkey. Plasma samples were collected at 30 min, 4 h, 24 h, 96 h, and 168 h post day 1 and day 8 doses and 30 min, 4 h, 24 h, and 72 hr post day 15 dose. Plasma concentration was measured by ELISA using a universal Fc capture and detection method. Plasma concentration values were interpolated from a standard curve and plotted using GraphPad PRISM. FIG. 9 depicts the pharmacokinetics in cynomolgus monkey of the dually masked molecules CI106 and CI139.

While the invention has been described in conjunction with the detailed description thereof, the foregoing description is intended to illustrate and not limit the scope of the invention, which is defined by the scope of the appended claims. Other aspects, advantages, and modifications are within the scope of the following.

1. A bispecific activatable antibody (BAA), wherein said BAA, when activated, specifically binds to two targets, and wherein said BAA, when not activated, comprises the following structure:
a) an IgG antibody (AB1) that specifically binds to a first target wherein the AB1 comprises:
i. two heavy chains (AB1 HCs) and two light chains (AB1 LCs); and
ii. two first prodomains, each comprising a first masking moiety (MM1) linked to a first cleavable moiety (CM1) in the N-terminal to C-terminal direction, wherein the carboxyl terminus of each first prodomain is linked to the amino terminus of each light chain of the AB1, wherein
the MM1 reduces or inhibits the binding of the AB1 to its target when the BAA is in an uncleaved state; and
the CM1 is a polypeptide that functions as a substrate for a first protease; and
b) two scFvs (AB2) that each specifically binds CD3ε, wherein each AB2 comprises:
i. a light chain variable region (VL) linked to a heavy chain variable region (VH), wherein the carboxyl terminus of each AB2 is linked to the amino terminus of each of the AB1 heavy chains, wherein
the VL comprises an amino acid sequence as set forth in SEQ ID NO: 1 or SEQ ID NO:4 and the VH comprises an amino acid sequence as set forth in SEQ ID NO: 2 or SEQ ID NO: 3; and
the VL is linked to the VH by a linker L3 comprising amino acid sequence SEQ ID NO: 108; and
two second prodomains, each comprising a second masking moiety (MM2) linked to a second cleavable moiety (CM2) in the N-terminal to C-terminal direction, wherein the carboxyl terminus of each second prodomain is linked to the amino terminus of each AB2, wherein
the MM2 reduces or inhibits the binding of the AB2 to CD3ε when the BAA is in an uncleaved state;
the MM2 comprises an amino acid sequence selected from the group consisting of SEQ ID NO: 12, SEQ ID NO: 105, SEQ ID NO: 106 and SEQ ID NO: 107; and
the CM2 is a polypeptide that functions as a substrate for a second protease;
or
ii. a light chain variable region (VL) linked to a heavy chain variable region (VH), wherein the carboxyl terminus of each AB2 is linked to the amino terminus of each of the AB1 heavy chains, wherein
the VL comprises an amino acid sequence as set forth in SEQ ID NO: 1 or SEQ ID NO:4 and the VH comprises an amino acid sequence as set forth in SEQ ID NO: 2 or SEQ ID NO: 3; and
the VL is linked to the VH by a linker L3 comprising amino acid sequence SEQ ID NO: 98; and
two second prodomains, each comprising a second masking moiety (MM2) linked to a second cleavable moiety (CM2) in the N-terminal to C-terminal direction, wherein the carboxyl terminus of each second prodomain is linked to the amino terminus of each AB2, wherein
the MM2 reduces or inhibits the binding of the AB2 to CD3ε when the BAA is in an uncleaved state;
the MM2 comprises an amino acid sequence selected from the group consisting of SEQ ID NO: 105, SEQ ID NO: 106 and SEQ ID NO: 107; and
the CM2 is a polypeptide that functions as a substrate for a second protease.

2. (canceled)

3. (canceled)

4. The BAA of claim 1, wherein in b)i, the MM2 comprises amino acid sequence SEQ ID NO: 12.

5. The BAA of claim 1, wherein the MM2 comprises amino acid sequence SEQ ID NO: 105.

6. The BAA of claim 1, wherein the MM2 comprises amino acid sequence SEQ ID NO: 106.

7. The BAA of claim 1, wherein the MM2 comprises amino acid sequence SEQ ID NO: 107.

8. The BAA of claim 1, wherein the AB1 binds a tumor target.

9. The BAA of claim 1, wherein the AB1 binds EGFR.

10. The BAA of claim 1, wherein the MM1 comprises an amino acid sequence selected from the group consisting of sequences presented in Table 7 or Table 8.

11. The BAA of claim 1, wherein the MM1 comprises an amino acid sequence selected from the group consisting of SEQ ID NO: 78 and SEQ ID NO: 85.

12. The BAA of claim 1, wherein the MM1 comprises amino acid sequence SEQ ID NO: 78.

13. The BAA of claim 1, wherein the MM1 comprises amino acid sequence SEQ ID NO: 85.

14. (canceled)

15. (canceled)

16. (canceled)

17. (canceled)

18. The BAA of claim 1, wherein the AB1 comprises an Fc region comprising an amino acid substitution in at least one of amino acid positions L234, L235, N297, and P331, as numbered by the EU index as set forth in Kabat, such that the BAA has reduced effector function.

19. The BAA of claim 18, wherein the AB1 comprises amino acid substitutions in at least two of amino acid positions L234, L235, and P331.

20. The BAA of claim 18, wherein the AB1 comprises amino acid substitutions at amino acid positions L234, L235, and P331.

21. The BAA of claim 18, wherein the AB1 comprises L234F, L235E, and P331S amino acid substitutions.

22. The BAA of claim 18, wherein the AB1 comprises an Fc region comprising an amino acid substitution at N297.

23. The BAA of claim 18, wherein the AB1 comprises L234F, L235E, P331S, and N297Q amino acid substitutions.

24. The BAA of claim 18, wherein the heavy chain of AB1 comprises an amino acid sequence selected from the group consisting SEQ ID NO: 115, SEQ ID NO: 116, SEQ ID NO: 117, SEQ ID NO: 118, SEQ ID NO: 119, SEQ ID NO: 120, SEQ ID NO: 121 and SEQ ID NO: 122.

25. The BAA of claim 18, wherein the heavy chain of AB1 comprises an amino acid sequence selected from the group consisting SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76.

26. The BAA of claim 1, wherein the first target is selected from the group consisting of the targets presented in Table 9.

27. The BAA of claim 1, wherein each of the first and second prodomains comprises a linker L1 and a linker L2 according to the first prodomain formula (MM1)-L1-(CM1)-L2 in the N-terminal to C-terminal direction and the second prodomain formula (MM2)-L1-(CM2)-L2 in the N-terminal to C-terminal direction.

28. A BAA comprising at least one of the following characteristics:
a) the BAA is CI138, wherein the BAA comprises a heavy chain amino acid sequence as set forth in SEQ ID NO: 155 or SEQ ID NO: 124 and a light chain amino acid sequence as set forth in SEQ ID NO: 132 or SEQ ID NO: 140;
b) the BAA is CI139, wherein the BAA comprises a heavy chain amino acid sequence as set forth in SEQ ID NO: 157 or SEQ ID NO: 126 and a light chain amino acid sequence as set forth in SEQ ID NO: 132 or SEQ ID NO: 140;
c) the BAA is CI158, wherein the BAA comprises a heavy chain amino acid sequence as set forth in SEQ ID NO: 161 or SEQ ID NO: 128 and a light chain amino acid sequence as set forth in SEQ ID NO: 132 or SEQ ID NO: 140; and
d) the BAA is CI140, wherein the BAA comprises a heavy chain amino acid sequence as set forth in SEQ ID NO: 159 or SEQ ID NO: 25 and a light chain amino acid sequence as set forth in SEQ ID NO: 132 or SEQ ID NO: 140.

29. (canceled)

30. (canceled)

31. (canceled)

32. An activatable antibody (AA), wherein said AA, when activated, specifically binds to a target, and wherein said AA, when not activated, comprises the following structure:
a. at least one scFv comprising a light chain variable region (VL) linked to a heavy chain variable region (VH), wherein the VL is linked to the VH by a linker L3 comprising amino acid sequence SEQ ID NO: 108; and
b. a prodomain comprising:
i. a masking moiety (MM) coupled to the scFv, wherein the MM reduces or inhibits the binding of the scFv to its target when the AA is in an uncleaved state; and
ii. a cleavable moiety (CM) coupled to the scFv, wherein the CM is a polypeptide that functions as a substrate for a protease.

33. (canceled)

34. An activatable antibody (AA) wherein said AA, when activated, specifically binds to a target, and wherein said AA, when not activated, comprises the following structure:
a) an antibody or antigen binding fragment thereof (AB) that specifically binds to CD3ε; and
b) a prodomain comprising:
i.a masking moiety (MM) coupled to the AB, wherein the MM reduces or inhibits the binding of the AB to the CD3ε when the AA is in an uncleaved state, wherein the MM comprises the amino acid sequence SEQ ID NO: 105 or SEQ ID NO: 106; or SEQ ID NO: 107 and
ii.a cleavable moiety (CM) coupled to the AB, wherein the CM is a polypeptide that functions as a substrate for a protease.

35. (canceled)

36. The BAA of claim 1, wherein the CM1 and CM2 comprise a substrate cleavable by a serine protease or an MMP.

37. (canceled)

38. (canceled)

39. (canceled)

40. (canceled)

41. (canceled)

42. (canceled)

43. (canceled)

44. (canceled)

45. A pharmaceutical composition comprising the BAA of claim 1 and a carrier.

46. The pharmaceutical composition of claim 45 comprising an additional agent.

47. The pharmaceutical composition of claim 46, wherein the additional agent is a therapeutic agent.

48. An isolated nucleic acid molecule encoding the BAA of claim 1.

49. A vector comprising an isolated nucleic acid molecule of claim 48.

50. A vector comprising at least one of the following characteristics:
a) a nucleic acid sequence encoding an amino acid sequence selected from the group consisting of SEQ ID NOs: 124 and 155;
b) a nucleic acid sequence encoding an amino acid sequence selected from the group consisting of SEQ ID NOs: 126 and 157;
c) a nucleic acid sequence encoding an amino acid sequence selected from the group consisting of SEQ ID NOs: 128 and 161; and
d) a nucleic acid sequence encoding an amino acid sequence selected from the group consisting of SEQ ID NOs: 124 and 159.

51. (canceled)

52. (canceled)

53. (canceled)

54. A cell comprising any one of the vectors of claim 49.

55. A cell comprising at least one of the following characteristics:
a) a nucleic acid sequence selected from the group consisting of SEQ ID NOs: 123 and 154;
b) a nucleic acid sequence selected from the group consisting of SEQ ID NOs: 125 and 156;
c) a nucleic acid sequence selected from the group consisting of SEQ ID NOs: 127 and 160; and
d) a nucleic acid sequence selected from the group consisting of SEQ ID NOs: 142 and 158.

56. (canceled)

57. (canceled)

58. (canceled)

59. A method of producing the BAA of claim 1 by culturing a cell under conditions that lead to expression of the BAA, wherein the cell comprises a nucleic acid molecule or a vector comprising a nucleic acid sequence that encodes an amino acid sequence selected from the group consisting of SEQ ID NOs: 124, 126, 128, 25, 155, 157, 161, 159, 140 and 132.

60. A method of treating, alleviating a symptom of, or delaying the progression of a disorder or disease comprising administering a therapeutically effective amount of the BAA of claim 1 or a pharmaceutical composition comprising the BAA of claim 1 and a carrier.

61. The method of claim 60, wherein the disorder or disease comprises disease cells expressing EGFR.

62. The method of claim 60, wherein the disorder or disease is cancer.

63. The method of claim 62, wherein the cancer is bladder cancer, breast cancer, cervical cancer, cholangiocarcinoma, colorectal cancer, endometrial cancer, esophageal cancer, gastric cancer, glioblastoma, head and neck cancer, liver cancer, lung cancer, ovarian cancer, pancreatic cancer, prostate cancer, renal cancer, sarcoma, squamous cell cancer, or skin cancer.

64. A method of inhibiting angiogenesis in a subject comprising administering a therapeutically effective amount of the BAA of claim 1 or a pharmaceutical composition comprising the BAA and a carrier.

65. The method of claim 60, wherein the method comprises administering an additional agent.

66. The method of claim 65, wherein the additional agent is a therapeutic agent.

67. A method of reducing damage to healthy tissue caused by an antibody binding to its target on healthy tissue as well as on diseased tissue, the method comprising administering to a subject in need thereof a BAA or a pharmaceutical composition comprising a BAA, wherein said BAA is the BAA of claim 1.

68. A method to improve tolerability of an antibody treatment comprising administering to a subject in need thereof a BAA or a pharmaceutical composition comprising a BAA, wherein said BAA is the BAA of claim 1.

69. A method to recruit T cells to tumor tissue comprising administering to a subject in need thereof a BAA or a pharmaceutical composition comprising a BAA, wherein said BAA is the BAA of claim 1.

70. A pharmaceutical composition comprising the AA of claim 32 and a carrier.

71. A pharmaceutical composition comprising the AA of claim 34 and a carrier.

72. An isolated nucleic acid molecule encoding the AA of claim 32.

73. An isolated nucleic acid molecule encoding the AA of claim 34.

74. A method of producing the AA of claim 32 by culturing a cell under conditions that lead to expression of the AA, wherein the cell comprises a nucleic acid molecule or a vector comprising a nucleic acid sequence that encodes an amino acid selected from the group consisting of SEQ ID Nos:124, 126, 128, 25, 155, 157, 161, 159, 140 and 132.

75. A method of producing the AA of claim 34 by culturing a cell under conditions that lead to expression of the AA, wherein the cell comprises a nucleic acid molecule or a vector comprising a nucleic acid sequence that encodes an amino acid selected from the group consisting of SEQ ID NOs: 124, 126, 128, 25, 155, 157, 161, 159, 140 and 132.

76. A method of treating, alleviating a symptom of, or delaying the progression of a disorder or disease comprising administering a therapeutically effective amount of the AA of claim 32 or a pharmaceutical composition comprising the AA and a carrier.

77. A method of treating, alleviating a symptom of, or delaying the progression of a disorder or disease comprising administering a therapeutically effective amount of the AA of claim 34 or a pharmaceutical composition comprising the AA and a carrier.
